(19) United States
(12) Patent Application Publication
(10) Pub. No.: US 2008/0085228 A1
(43) Pub. Date: Apr. 10, 2008

(54) Image reading device

(75) Inventors: Atsushi Yamazaki, Kanagawa (JP); Hiroaki Yasuda, Kanagawa (JP); Shinichiro Fukui, Kanagawa (JP); Satoshi Arakawa, Kanagawa (JP); Yuichi Hosoi, Kanagawa (JP); Tomohiko Sato, Kanagawa (JP); Hiroyuki Kohda, Kanagawa (JP); Yasuhiki Goto, Tokyo (JP); Masayuki Iwasaka, Tokyo (JP)

Correspondence Address: BIRCH STEWART KOLASCH & BIRCH, PO BOX 747, FALLS CHURCH, VA, 22040-0747, US

(73) Assignee: FUJIFILM Corporation, Tokyo (JP)

(21) Appl. No.: 11/902,962

(22) Filed: Sep. 26, 2007

Related U.S. Application Data
(63) Continuation-in-part of application No. 11/528,403, filed on Sep. 28, 2006.

(30) Foreign Application Priority Data
Sep. 30, 2005 (JP) 2005-288834
Sep. 27, 2006 (JP) 2006-262266
Mar. 27, 2007 (JP) 2007-082548
Mar. 27, 2007 (JP) 2007-082547
Mar. 27, 2007 (JP) 2007-082546
Jul. 24, 2007 (JP) 2007-191791

(51) Int. Cl. A61L 2/00; A61L 2/04; A61L 2/10; A61L 2/16; A61L 2/20

(52) U.S. Cl. 422/291

(57) ABSTRACT

An image reading device includes at least a disinfection unit that administers a disinfection treatment to either an imaging medium such as a radiation image conversion panel or a protective member that protects at least an imaging surface of the imaging medium. The system of disinfection of the radiation image conversion panel is a disinfection treatment by the disinfection unit that is preferably at least one of heat treatment, ultraviolet ray irradiation treatment, chemical coating treatment and gas treatment.

### CROSS-REFERENCE TO RELATED APPLICATION

This application claims priority under 35 USC 119 from Japanese Patent Application No. 2005-288834 filed Sep. 30, 2005, No. 2007-191791 filed Jul. 24, 2007, Nos. 2007-082546, 2007-082547, 2007-082548 filed Mar. 27, 2007 and No. 2006-262266 filed Sep. 27, 2006, respectively. This application is a continuation-in-part of U.S. application Ser. No. 11/528,403, the disclosure of which is incorporated by reference herein.

### BACKGROUND

1. Field of the Invention

The present invention relates to an image reading device that reads a radiation image carried on an imaging medium.

2. Related Art

Recently, with a growing interest in measures against infectious diseases among medical professionals, there is desired an apparatus which disinfects an imaging medium such as a radiation panel. Moreover, particularly, since a radiation image conversion panel or radiation image conversion film for dental application is handled in the mouth, a likelihood where body fluid such as saliva of a patient is adhered thereto enhances this desire.

As a disinfection apparatus used for medical instruments, there is proposed an apparatus which disinfects by using an oil of a high temperature (for example, refer to Japanese Patent Application Laid-Open (JP-A) No. 2005-131359). However, the apparatus has a safety issue since an oil is used, and it is unsuitable for disinfecting a radiation image conversion panel that is easily deformed in a structure of the apparatus.

Consequently, in practice, disinfection is performed by wiping with alcohol such as ethanol. As a result, there is a problem in that disinfection becomes uneven, incomplete, and inefficient, since disinfection is manually performed one by one.

### SUMMARY

The present invention has been made in view of the above circumstances and provides an image reading device.

A first aspect of the present invention provides an image reading device, comprising a disinfection unit that administers a disinfection treatment to an imaging medium carrying a radiation image or to a protective member covering at least an imaging surface of the imaging medium and an image reading unit that reads the radiation image carried by the imaging medium either after or before the disinfection treatment by the disinfection unit.

### BRIEF DESCRIPTION OF THE DRAWINGS

FIG. 1 is a block diagram of the inside of an image reading device according to a first embodiment.

FIG. 2 is a diagram of the outside of an image reading device according to the first embodiment.

FIG. 3 is a diagram of the inside of an image reading device according to another aspect of the first embodiment.

FIG. 4 is a diagram of the inside of an image reading device according to another aspect of the first embodiment.

FIG. 5 is a diagram of the inside of an image reading device according to another aspect of the first embodiment.

FIG. 6 is a sectional side view showing a schematic configuration of an image reading device according to a second embodiment.

FIG. 7 is a sectional side view showing a schematic configuration of an image reading device according to the second embodiment.

FIG. 8A is a perspective view showing an imaging plate and a protective case in which the imaging plate is enclosed.

FIG. 8B is a sectional view showing an imaging plate and a protective case in which the imaging plate is enclosed.

FIG. 9 is a sectional side view showing a schematic configuration of a disinfection mechanism with which an image reading device according to the second embodiment is equipped.

FIG. 10 is a sectional side view showing a schematic configuration of a modified example of the disinfection mechanism shown in FIG. 9.

FIG. 11 is a sectional side view showing a schematic configuration of a first modified example of the disinfection mechanism shown in FIG. 9.

FIG. 12 is a sectional side view showing a schematic configuration of a modified example of the disinfection mechanism shown in FIG. 11.

FIG. 13 is a sectional side view showing a schematic configuration of a second modified example of the disinfection mechanism shown in FIG. 9.

FIG. 14 is a sectional side view showing a schematic configuration of a third modified example of the disinfection mechanism shown in FIG. 9.

FIG. 15 is a partially enlarged sectional side view showing an image reading mechanism with which the image reading device shown in FIG. 6 is equipped.

FIG. 16A is a sectional side view showing a protective case enclosure mechanism with which the image reading device shown in FIG. 6 is equipped.

FIG. 16B is a sectional view of an imaging plate and a protective case in which the imaging plate is enclosed.

FIG. 17 is a sectional side view showing a schematic configuration of a contamination-prevention pack enclosure mechanism with which the image reading device shown in FIG. 6 is equipped.

FIG. 18 A is a sectional side view showing a schematic configuration of a modified example of the contamination-prevention pack enclosure mechanism shown in FIG. 17.

FIG. 18 B is a sectional side view showing a schematic configuration of a modified example of the contamination-prevention pack enclosure mechanism shown in FIG. 17.

FIG. 19 is a sectional side view showing an image reading device according to a third embodiment.

FIG. 20 is a sectional side view showing an image reading device according to the third embodiment.

FIG. 21 is a sectional side view showing a schematic configuration of a cleaning mechanism with which an image reading device according to the third embodiment is equipped.

FIG. 22A is a sectional side view showing a schematic configuration of a first modified example of the cleaning mechanism shown in FIG. 21.

FIG. 22B is a sectional side view showing a schematic configuration of the first modified example of the cleaning mechanism shown in FIG. 21.

FIG. 23 is a sectional side view showing a schematic configuration of a second modified example of the cleaning mechanism shown in FIG. 21.

FIG. 24 is a sectional side view showing a schematic configuration of an image reading device according to a fourth embodiment.

FIG. 25A is a plan view showing a schematic configuration of a protective case removal mechanism with which an image reading device according the fourth embodiment is equipped.

FIG. 25B is a sectional view along the line B-B in FIG. 25A, showing a schematic configuration of a protective case removal mechanism with which an image reading device according the fourth embodiment is equipped.

FIG. 26A is a plan view showing a schematic configuration of the protective case removal mechanism with which an image reading device according the fourth embodiment is equipped.

FIG. 26B is a sectional view along the line B-B in FIG. 26A, showing a schematic configuration of a protective case removal mechanism with which an image reading device according the fourth embodiment is equipped.

FIG. 27A is a sectional view showing a schematic configuration of a modified example of the protective case removal mechanism shown in FIGS. 25 and 26.

FIG. 27B is a sectional view showing a schematic configuration of a modified example of the protective case removal mechanism shown in FIGS. 25 and 26.

FIG. 27C is a sectional view showing a schematic configuration of a modified example of the protective case removal mechanism shown in FIGS. 25 and 26.

FIG. 28 is a sectional side view showing a schematic configuration of an image reading device according to a fifth embodiment.

FIG. 29 is a sectional side view showing a schematic configuration of an image reading device according to a sixth embodiment.

FIG. 30 is a sectional side view showing a schematic configuration of an image reading device according to a seventh embodiment.

FIG. 31 is a sectional side view showing a schematic configuration of an image reading device according to an eighth embodiment.

FIG. 32 is a sectional side view showing a schematic configuration of an erasing and disinfection mechanism, with which an image reading device according to the eighth embodiment is equipped.

### DETAILED DESCRIPTION OF THE INVENTION

The image reading device of the present invention comprises a disinfection unit which applies a disinfection treatment to at least a radiation image conversion panel, a radiation image conversion film (imaging medium), and/or a light shielding bag (protective member) that can be used to wrap such a radiation image conversion panel or radiation image conversion film therein (referred to sometimes below as “items to be disinfected”).

The disinfection treatment by the disinfection unit is preferably, from a practical viewpoint, at least a treatment selected from a heat treatment, an ultraviolet irradiation treatment, a chemical application treatment, a gas treatment, with heat treatment and ultraviolet irradiation being more preferable. If the disinfection treatment is a heat treatment, the temperature of the heat treatment is preferably 60° C. to 200° C., and more preferably 90 to 120° C. Moreover, the time for the heat treatment is preferably 1 second to 10 minutes, and more preferably 10 seconds to 5 minutes.

For example, in order to kill botulinum toxins it is possible to carry out heat treatment with heating at 120° C. for about 30 minutes.

If disinfection is performed by the heat treatment, the disinfection unit preferably comprises a temperature control unit. As to the temperature control unit, a normal temperature control device may be used. By providing the temperature control unit, the items for disinfection can be set within the abovementioned temperature range.

The heating unit is not specifically limited and may be for example a unit which supplies hot air to the radiation image conversion panel, or a unit using an infrared heater or a far infrared heater. However, from the viewpoint of temperature controllability and safety, the heat treatment is preferably performed using either one of an infrared heater and a far infrared heater. The power when using the infrared heater or the far infrared heater is preferably 50 to 1000 W. As another way to carry out heat treatment, microwaves can be used. In this case, at least waves in the frequency range 300 MHz to 30 GHz should be included. Using microwaves is highly safe, and the speed of heating is fast and heating efficiency high. There is also the merits that it is possible to uniformly heat complicated shaped objects and the operation and control thereof is simple.

Moreover, the ultraviolet irradiation unit for the disinfection treatment is a unit which irradiates ultraviolet light by an ultraviolet lamp onto the items to be disinfected. However, if ultraviolet light is over-irradiated, the phosphor layer might be sensitized. Therefore it is necessary to appropriately adjust the irradiation time. With the use of ultraviolet light irradiation there are the merits that the operation thereof is simple, it is possible to maintain a hygienic environment, and it is highly safe.

The irradiation energy of ultraviolet light in the disinfection process is preferably 0.04 J/cm² or above. Further, it is preferable to include wavelengths at least in the range of 250 to 280 nm. In particular it is preferable to include the wavelength 254 nm, known as the wavelength with the strongest disinfecting power. Further, when considering the prevention of ultraviolet light fogging during erasing, it is preferable to carry out the processing for erasing of the image data using erasing unit 39 after carrying out the disinfection treatment.

A first embodiment of chemical application treatment serving as another unit of the disinfection unit, includes providing an immersion tank filled with an agent, and a treatment of immersing the item to be disinfected into the immersion tank. In the case of the immersion treatment, the immersion time is preferably about 1 to 600 seconds, and the immersion may be performed appropriately for a plurality of times. In addition to the immersion treatment, a unit which spray-coats an agent may be employed. The agent includes: alcohol such as ethanol; aldehyde such as glutaraldehyde; and peracetic chlorine.

Further, as a second embodiment of chemical application treatment is where an agent is applied by passing the item to be disinfected between a pair of rollers impregnated with one of the above agents. Plural pairs of the rollers may be arranged either in series or arranged intermittently.

For disinfection using a gas (gas treatment), ethylene oxide, ozone can be blown onto the items to be disinfected. It is possible to carry out processing using ethylene oxide at a temperature close to room temperature. Ozone can demonstrate excellent effects in breaking down germs and organic matter, because of its strong oxidizing power.

As other examples of disinfecting methods, radiation irradiation unit can be given. These include the irradiation of electromagnetic waves and rays with wavelengths below that of the ultraviolet region, such as γ-rays and X-rays, onto the items to be disinfected. These methods are particularly effective when the carrying out of heat treatment is difficult.

The disinfection system of the present invention preferably comprises an image reading unit which reads out an image on the radiation image conversion panel and/or the radiation image conversion film. The image reading unit provides an advantage in that the disinfection treatment and the image reading process can be realized in one system.

From the viewpoint of protecting the phosphor layer, the radiation image conversion panel and/or the radiation image conversion film may be formed with a protective layer. If the radiation image conversion panel formed with the protective layer is subjected to a disinfection treatment by means of heating, it may be deformed and thus becomes deficient depending on its material. Consequently, if such a disinfection treatment by means of heating is applied, the thermal shrinkage rate (JISC2151 which is incorporated herein by reference, at 150° C. for 30 minutes) of the protective layer is preferably 1% or less, and more preferably 0.01 to 0.8%. If the thermal shrinkage rate is 1% or less, the deformation due to thermal shrinkage can be prevented.

The protective layer of the radiation image conversion panel and/or radiation image conversion film is preferably subjected to a heat treatment of 60° C. or more, at least either before or at the time of its formation. By applying such a heat treatment, the deformation due to heating can be prevented even if the disinfection treatment by means of heating is performed.

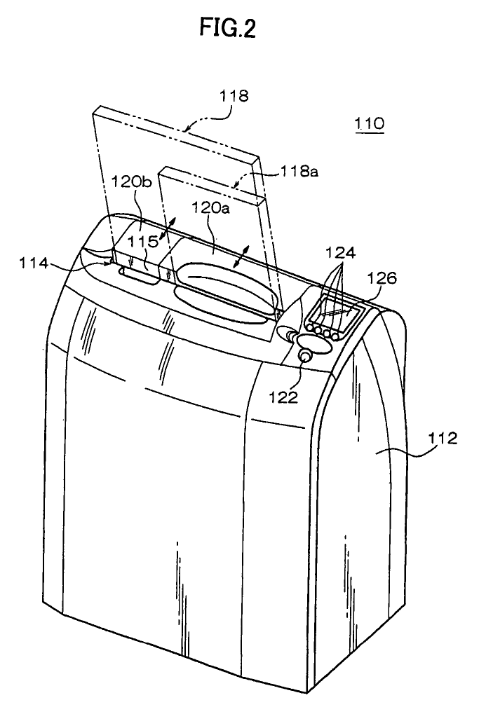

Radiation image conversion films are generally films of approximately 3 cm×4 cm of a form which can be used in the taking of dental internal oral X-ray images. Light shielding bags that can be used to wrap such radiation image conversion films are light shielding bags of about the same size for wrapping radiation image conversion films therein, and after wrapping they can be sealed with double-sided tape or the like to give a sealed envelope state. Further, examples of possible embodiments are disclosed in the Examples and FIGS. 2 to 4 of Japanese Patent Application Laid-Open No. S64-49032 or Japanese Patent Publication (JP-B) No. 6-100791.

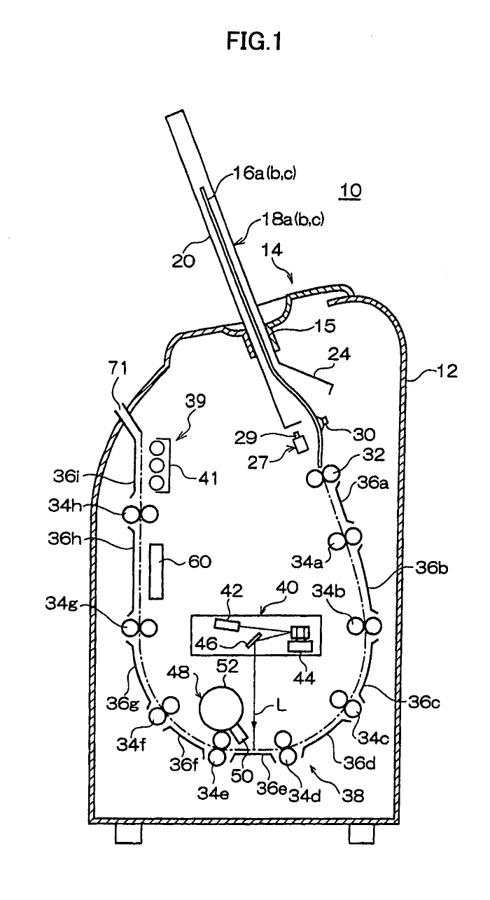

Next is a description of the first embodiment of the image reading device of the present invention, with reference to FIG. 1.

The image reading device 10 comprises a cassette loading portion 14 on the top of a casing 12. Through a loading inlet 15 formed in this cassette loading portion 14, is loaded an image recording medium having radiation image data cumulatively recorded therein, such as a cassette 18a (18b, 18c) housing an image conversion panel 16a (16b, 16c). In a case of a radiation image conversion panel used for dental application, the cassette may not be used in some cases. Specifically, a radiation image conversion panel stored in a predetermined bag is taken out and subjected to various treatments.

The width of the cassette 18b is narrower than that of the cassette 18a. The width of the cassette 18c is narrower than that of the cassette 18b. The width of the radiation image conversion panel 16b stored in the cassette 18b is narrower than that of the radiation image conversion panel 16a stored in the cassette 18a. The width of the radiation image conversion panel 16c stored in the cassette 18c is narrower than that of the radiation image conversion panel 16b stored in the cassette 18b.

In the description hereunder, although the cassette 18a and the radiation image conversion panel 16a are used, the description is similarly applied to the cassettes 18b and 18c and the radiation image conversion panels 16b and 16c.

The cassette 18a comprises a mainframe 20 which houses the radiation image conversion panel 16a, and a lid member 24 which forms an opening portion for putting in/taking out the radiation image conversion panel 16a.

In the vicinity of the loading inlet 15 inside of the image reading device 10 is arranged: a lock release mechanism 27 which releases locking of the lid member 24 of the cassette 18a; a suction cup 30 which attracts the radiation image conversion panel 16a and takes it out from the cassette 18a with the lid member 24 open; and a roller pair 32 which interposes therebetween the radiation image conversion panel 16a that has been taken out by the suction cup 30, and conveys it. The lock release mechanism 27 has a lock release pin 29 for releasing a cassette lock unit (not shown) that is inserted into the cassette 18a.

Lined up with the roller pair 32, a plurality of conveying roller pairs 34a to 34h and a plurality of guide plates 36a to 36i are arranged, constituting a curved conveying path 38.

In the approximate center of the image reading device 10 is arranged a scanning unit 40 which emits laser beams L serving as exciting light and scans the radiation image conversion panel 16a. The scanning unit 40 comprises: a laser oscillator 42 which outputs a laser beam L; a polygon mirror 44 serving as a rotating polygon mirror which deflects the laser beam L in the main scanning direction of the radiation image conversion panel 16a; and a reflection mirror 46 which reflects the laser beam L to guide to the radiation image conversion panel 16a passing through on the guide plate 36e.

Between the conveying roller pair 34e and the scanning unit 40 is arranged a reading unit 48. The reading unit 48 comprises: a light-converging guide 50 having one end arranged in the vicinity of the radiation image conversion panel 16a on the guide plate 36e; and a photomultiplier 52 which is connected to the other end of the light-converging guide 50, and converts photo-stimulated luminescence light obtained from the radiation image conversion panel 16a into electric signals.

Moreover, between conveying roller pairs 34e and 34h is provided a disinfection unit 60. Here, the radiation image conversion panel applied with the disinfection treatment is conveyed to the outlet 71 and taken out.

The image reading device comprising the disinfection unit 60 operates as described below. Firstly, the cassette 18a which houses the radiation image conversion panel 16a having the radiation image data recorded therein, is supplied to the image reading device 10. The cassette 18a is loaded into the loading inlet 15 of the cassette loading portion 14 having the lid member 24 faced downward. The locking of the lid member 24 is released through the lock release mechanism 27.

Between the roller pair 34h and the outlet 71 is arranged an erasing unit 39 for erasing the radiation image data remaining on the radiation image conversion panel 16a having the read processing completed. The erasing unit 39 has an erasing light source 41 such as a cold-cathode tube which outputs erase light.

Next, the radiation image conversion panel 16a in the cassette 18a is taken out from the cassette 18a under the suction effect of the suction cup 30. The tip of the radiation image conversion panel 16a that has been taken out from the cassette 18a is interposed between the roller pair 32, and at the same time the attraction and the holding of the radiation image conversion panel 16a by the suction cup 30 are released.

As a result, the radiation image conversion panel 16a is conveyed vertically downward under the rotation effect of the roller pair 32. This radiation image conversion panel 16a is conveyed by the curved conveying path 38 comprising the conveying roller pairs 34a to 34h and the guide plates 36a to 36i.

When the conveying roller pairs 34b and 34c are synchronously driven and thereby the radiation image conversion panel 16a is conveyed to a pull-over device 54 (not shown), the radiation image conversion panel 16a is released from being interposed between the conveying roller pair 34b and 34c.

The radiation image conversion panel 16a having the pull-over processing completed as described above, is conveyed for sub-scanning between the conveying roller pairs 34d and 34e, and the laser beam L emitting from the scanning unit 40 scans over the radiation image conversion panel 16a in the main scanning direction orthogonal to the sub-scanning direction. That is, the laser beam L output from the laser oscillator 42 is reflected and deflected by the polygon mirror 44 which rotates at high speed, and is then guided to the radiation image conversion panel 16a through the reflection mirror 46.

On the other hand, the radiation image conversion panel 16a irradiated with the laser beam L outputs photo-stimulated luminescence light corresponding to the cumulatively recorded radiation image data. This photo-stimulated luminescence light is guided to the photomultiplier 52 constituting the reading unit 48 through the light-converging guide 50 that is arranged in the vicinity along the main scanning direction of the radiation image conversion panel 16a.

The radiation image conversion panel 16a in which the radiation image data has been read out in this manner, is disinfected by the disinfection unit and conveyed to the conveying roller pair 34h side. Thereafter, erasing unit 39 drives and controls erasing light source 41, and radiation image information remaining in radiation image conversion panel 16a is subjected to an erasing process with an erasing light outputted from erasing light source 41. The method for erasing a remaining radiation image of the description of JP-A No. 11-352615 may be referred to.

Then, the radiation image conversion panel 16a is conveyed to the outlet 71 and taken out. If the disinfection unit controls the temperature by the temperature control unit in the heat treatment unit, the surface temperature measurement method when the temperature is controlled, is preferably performed by bringing the radiation image conversion panel into contact with a thermocouple.

The disinfected radiation image conversion panel 16a that has been taken out, is supplied for image capturing of the next radiation image data.

In addition to the above, aspects of the image reading device according to the first embodiment of the present invention are such as the following.

(1) A first embodiment is an embodiment in which the radiation panel or radiation image conversion film which has had images taken thereon is wrapped within a light shielding bag, and this sealed. In this sealed state it is conveyed to the disinfection unit of the disinfection system, and here disinfection treatment is carried out. Specifically, the light shielding bag is conveyed to the disinfection unit of the image reading device using conveying rollers, and here the disinfection treatment is carried out by the irradiation of ultraviolet light from an ultraviolet light source. After this, the light shielding bag is conveyed by rollers to a light shielding bag opening unit. While one edge of the light shielding bag is held down by a holding member the other end of the light shielding bag is opened by use of an opening means such as a cutter or the like, and the radiation panel or the radiation image conversion film is taken out, and appropriately conveyed to the image reading unit. The light shielding bag from which the radiation panel or the radiation image conversion film has been removed is disposed of appropriately.

By this embodiment it is possible to disinfect in a sealed condition, and avoid adherence of bodily fluids or germs to the radiation image conversion panel or radiation image conversion film when the light shielding bag is opened. The disinfection treatment of the disinfection unit can be carried out, as described above, by heat treatment, chemical application treatment, gas-disinfection treatment (as is also the case in the embodiments that follow).

(2) A second embodiment is an embodiment in which the radiation image conversion film before it has had images taken thereon is wrapped within a light shielding bag, and this sealed. In this sealed state it is conveyed to the disinfection unit of the image reading device, and here disinfection treatment is carried out. Specifically, the light shielding bag is conveyed to the disinfection unit of the image reading device using conveying rollers, and here heat treatment (disinfection treatment) is carried out by a heater. After this, the light shielding bag is discarded. By this embodiment it is possible, because disinfection is completed in the sealed condition, it can be loaded into the mouth or into the body of a patient just as it is.

(3) A third embodiment is an embodiment in which the radiation image conversion panel and/or radiation image conversion film is conveyed to the disinfection unit of the image reading device, and here disinfection treatment is carried out. Specifically, radiation image conversion panel and/or radiation image conversion film is conveyed to the disinfection unit using conveying rollers, and here the radiation image conversion panel and/or radiation image conversion film is passed through the nip of a pair of sponge rollers impregnated with an agent, thereby carrying out disinfection treatment. After this, appropriate conveyance thereof is made to the image reading unit. By this embodiment it is possible to disinfect body fluids and germs that have adhered when removing from the light shielding bag, and thereby avoid contagion from the radiation image conversion panel or radiation image conversion film.

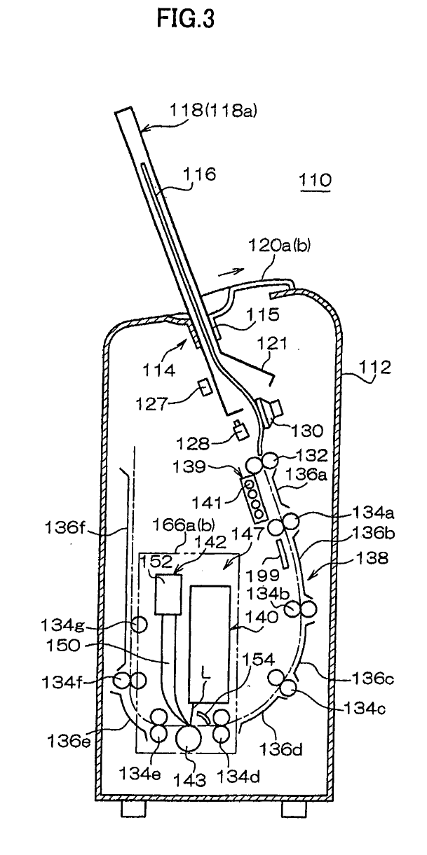

(4) A fourth embodiment will now be explained with reference to FIGS. 2 and 3.

In FIG. 2, a view is shown of the external appearance of an image reading device 110 common to embodiments 4 to 6, in FIG. 3 the internal structure is shown.

In FIG. 2, the image reading device 110 is provided with a cassette loading portion 114 at the top portion of casing 112, and the cassette 118 (118a) containing the radiation image conversion panel with the radiation image information stored and recorded thereon is loaded into the loading inlet 115 formed in the cassette loading portion 114. The cassette 118a is smaller in size that the cassette 118.

The radiation image conversion panel is a panel having a storage phosphor layer which, when irradiated with radiation (X-rays, α-rays, β-rays, γ-rays, electron beams, ultraviolet rays or the like) a portion of the radiation energy is stored, and then afterwards, with the irradiation by excitation light, of laser light or visible light and the like, stimulated phosphorescence in response to the stored energy is displayed. When the remaining energy is erased by irradiation with erasing light including light in the wavelength of the excitation light of the phosphor, the panel can be reused.

The cassette loading portion 114 has cover portion members 120a, 120b which are independently displaceable in the direction of the arrow. When the large size cassette 118 is loaded, the cover portion members 120a, 120b both displace and the entire loading inlet 115 is opened. When the small cassette 118a is loaded, only the cover portion member 120a displaces and a portion of the loading inlet 115 is opened. By this arrangement, the ingression of dust into the inner portion of the image reading device 110 can be repressed. On a side portion of the cassette loading portion 114, a power source button 122, an operating button 124, a display portion 126 and the like are disposed.

In FIG. 3, at an internal portion of the image reading device 110 near to the loading inlet 115, there is: a panel information readout portion 127 for reading out various information, such as the size, sensitivity and the like, identification number and the like (referred to as “panel information” below) of the radiation image conversion panel 116 accommodated in the loaded cassette 118 (118a); a lock release mechanism 128 for releasing the lock of the lid portion member 121 of the cassette 118 (118a); a suction pad 130 for suctioning and taking out the radiation image conversion panel 116 from the cassette 118 (118a) with opened lid portion member 121; and nip rollers 132 for nipping and conveying the radiation image conversion panel 116 that has been taken out by the suction pad 130.

The panel information readout portion 127 configured with a read-out unit, such as a bar code reader, RFID or the like, reads out the panel information recorded on a bar-code, IC chip or the like mounted on the cassette 118 (118a) or the radiation image conversion panel 116.

Plural conveying rollers 134a to 134g and plural guide plates 136a to 136f are disposed in conjunction to the nip rollers 132, and these configure the curved conveying path 138. The curved conveying path 138, after extending in a downward direction from the cassette loading portion 114, becomes substantially horizontal at the lowest portion thereof, then extends substantially vertically upwards. By this configuration the image reading device 110 can be made compact.

Between the nip rollers 132 and the conveying rollers 134a, an erasing unit 139 is disposed for erasing the radiation image information remaining in the radiation image conversion panel 116 after the read-out process has been completed. The erasing unit 139 has plural erasing light sources 141 made up from cold cathode tubes that emit erasing light.

Between the conveying rollers 134d and 134e which are arranged at the lowest portion of the curved conveying path 138, a platen roller 143 is disposed. At the upper portion of the platen roller 143, accommodated in a housing 145, is disposed a scanning unit 147 for reading out the radiation image information stored and recorded in the radiation image conversion panel 116.

Read-out section 166 a (b) is explained below. The scanning unit 147 is provided with: an excitation portion 140, for guiding the light of the excitation light laser beam L, scanning the radiation image conversion panel 116; and an image information read-out portion 142, for reading out the photo-stimulated luminescence light related to the radiation image information that is output from the excitation due to the laser beam L. The image information read-out portion 142 is provided with a photomultiplier 152, for converting the photo-stimulated luminescence light obtained from the radiation image conversion panel 116 into an electrical signal, the photomultiplier 152 being connected on one edge portion to a light guide 150 disposed in the vicinity of the radiation image conversion panel 116 above the platen roller 143, and on the other edge portion to light guide 150. In order to increase the collecting efficiency of the accelerated phosphorescent light, a light-converging mirror 154 is placed in the vicinity of one end of the light guide.

In the fourth embodiment is shown an example of carrying out heat treatment using a heater 199 provided as a disinfection unit at the lower side of the erasing unit 139.

The image reading device 110 of this embodiment of the invention is basically configured as above, and the operation thereof will now be explained.

First, lid portion member 121 is moved down and the cassette 118 (118a) accommodating the radiation image conversion panel 116 with the radiation image information stored and recorded thereon is loaded at the loading inlet 115 of the cassette loading portion 114.

Next, the panel information read-out portion 127 reads out the panel information including the type discriminator of the radiation image conversion panel 116 and the like from the cassette 118 (118a) or from the radiation image conversion panel 116 accommodated in the cassette 118 (118a).

When panel information can be read out, the lock release mechanism 128 is driven, the locked condition of the lid portion member 121 is released and lid opened. Next, the suction pad 130 suctions the radiation image conversion panel 116, and pulls out the radiation image conversion panel 116 from the cassette 118 (118a) and supplies it between the nip rollers 132. The radiation image conversion panel 116, nipped between the nip rollers 132, is conveyed past the disinfection unit 139, and conveyed to below the lower portion of the scanning unit 147 via the curved conveying path 138 formed from the conveying rollers 134a to 134b and guide plates 136a to 136f.

The radiation image conversion panel 116 is conveyed in a substantially horizontal direction in the sub-scanning direction by the conveying rollers 134d and 134e. Here, the laser beam L emitted from the excitation unit 140 is guided to the radiation image conversion panel 116 supported on the lower face portion by the platen roller 143, and the radiation image conversion panel 116 is scanned in the main direction.

The radiation image information that is stored and recorded in the radiation image conversion panel 116 is excited by the irradiation with the laser beam L, and is output as photo-stimulated luminescence light. This photo-stimulated luminescence light is directly illuminated into the lower end portion of the light guide 150 configuring the image information read-out portion 142, disposed adjacent to and along the main scanning direction of the radiation image conversion panel 116, or illuminated into the same via a light-converging mirror 154. The photo-stimulated luminescence light that is illuminated into the light guide 150 is guided to the upper end portion photomultiplier 152, being internally reflected multiple times. The photomultiplier 152 converts the photo-stimulated luminescence light illuminated therein to an electrical signal, and in this way the radiation image information that is stored and stored in the radiation image conversion panel 116 is read out.

Next, the radiation image conversion panel 116 from which the radiation image information has been read out is conveyed from the scanning unit 147 again to the erasing unit 139 side via the curved conveying path 138. Then, the disinfection treatment is carried out by a heater 199 provided at the adjacent side of the erasing unit 139. After the disinfection treatment is carried out the radiation image conversion panel 116 is conveyed to the erasing unit 139.

The erasing unit 139 drives and controls the erasing light sources 141 based on the erasing light amount arranged according to the panel information read out by the panel information read-out portion 127 and the radiation image information read out by the image information read-out portion 142. By the erasing light output from the erasing light sources 141, erasing processing is carried out of the radiation image information that remains in the radiation image conversion panel 116.

The radiation image conversion panel 116 from which the remaining radiation image information has been erased is accommodated in the cassette 118 (118a) loaded into the cassette loading portion 114, after lid closure with the lid portion member 121, it is removed from the cassette loading portion 114 and can be supplied for the next image exposure.

In the above, description of a case in which read-out of the radiation image information has been made by scanning of the radiation image conversion panel 116 with the laser beam L, however, it is applicable also to, for example, recording image information by scanning a recording medium with a laser beam L modulated according to the image information.

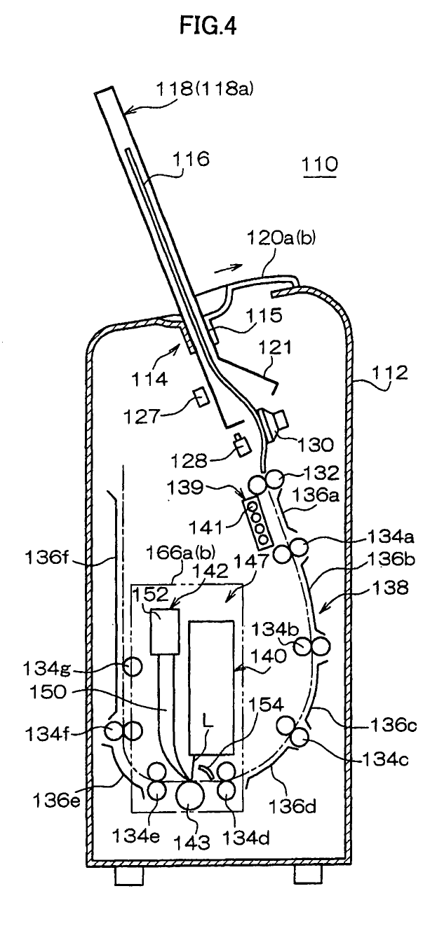

Further, in a fifth embodiment, as is shown in FIG. 4, it is configured so that a heater is not provided and the erasing unit 139 combines the function of the disinfecting system. That is to say this is an embodiment in which, after the reading out of the radiation image information, around the time of the erasing processing of the remaining radiation image information in the radiation image conversion panel 116, or during the processing, disinfection treatment can be carried out by the output of erasing light from the erasing light sources 141. According to the fifth embodiment it is possible to selectively carry out erasing processing and disinfection treatment, giving superior operating characteristics.

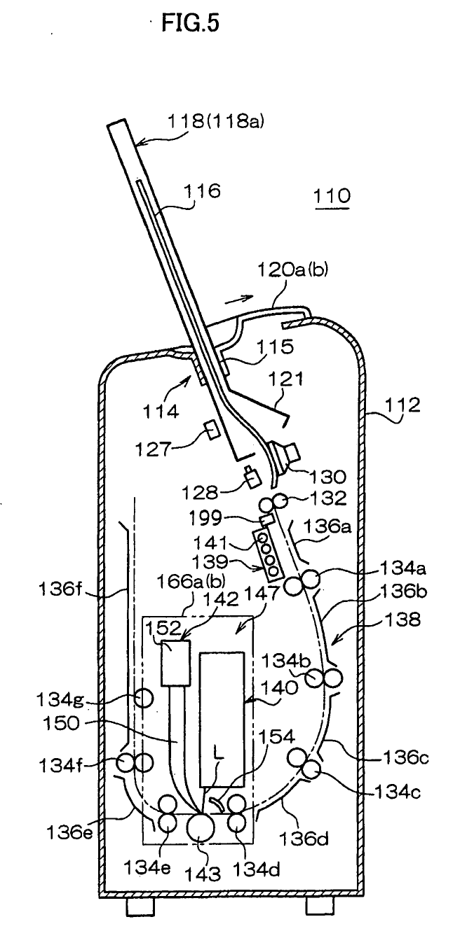

Further, in a sixth embodiment, as is shown in FIG. 5, there is an embodiment in which heat treatment using the heater 199 as the disinfection unit of the fourth embodiment is provided further to the upper side than the erasing unit 139. That is to say, after the reading out of the radiation image information, after carrying out the erasing processing on the remaining radiation image information of the radiation image conversion panel 116 by the output of erasing light from the erasing light sources 141, disinfection treatment is carried out by the heater 199.

The radiation image conversion panel and radiation image conversion film applied to the image reading device of the present invention has a structure, for example where an interlayer, a phosphor layer, a protective layer, and the like are sequentially formed on a support. Hereunder is a description of materials and the like of the respective layers.

(Support)

For the support, a material such as PET, polycycloolefine, PEN (polyethylene naphthalate), PVA (polyvinyl alcohol), a nanoalloy polymer of PET and PEI (polyetherimide), or a transparent aramid is preferably used. In particular, it is desirably a base material having a glass transition temperature (Tg) of 85° C. or more, and preferably 100° C. or more. It is preferably made from a material, such as polycycloolefine, PEN (polyethylene naphthalate), PVA (polyvinyl alcohol), a nanoalloy polymer of PET and PEI (polyetherimide), or a transparent aramid having a glass transition temperature of 85° C. or more. Furthermore, it is more preferably made from a material, such as polycycloolefine, PEN (polyethylene naphthalate), a nanoalloy polymer of PET and PEI (polyetherimide), or a transparent aramid having a glass transition temperature of 100° C. or more.

(Interlayer)

For the interlayer, a transparent high molecular material such as: a cellulose derivative such as acetylcellulose or nitrocellulose; or a synthesized high molecular material of polymethyl methacrylate, polyvinyl butyral, polyvinyl formal, polycarbonate, polyvinyl acetate, vinyl chloride/vinyl acetate copolymer, fluororesin, polyethylene, polypropylene, polyester, acrylic, polyparaxylylene, PET, a hydrochlorinated rubber, a vinylidene chloride copolymer, or the like may be used. These synthesized high molecular materials forming the interlayer may be used as a polymer or a monomer, but are preferably a material which crosslinks by irradiation of heat, visible light, UV light, electron beams, or the like.

If the interlayer is provided on the support, in order to improve the adhesiveness, a coupling agent such as a silane coupling agent and a titanate coupling agent is preferably added. Furthermore, in order to improve the coating property of the interlayer composition and the physical properties of the cured thin film, and to apply a photosensitivity to the coated film, there may be contained various additives for example various polymers and monomers having hydroxyl groups, colorants such as pigments and dyes, a stabilizer such as an anti-yellowing agent, an anti-aging agent, and an ultraviolet absorber, a heat acid generator, a photosensitive acid generator, a surfactant, a solvent, a cross-linking agent, a hardening agent, a polymerization inhibitor, and the like, according to the purpose.

Moreover, in order to improve the durability and to prevent bleeding and unevenness, the interlayer may contain organic or inorganic powder. If the powder is contained, it is preferably about 0.5 to 60% by weight with respect to the weight of the interlayer. The powder is preferably one that has an absorption in a specific bandwidth, such as ultramarine blue, or white powder which does not exhibit a specific absorption in a wavelength region of generally 300 to 900 nm. The volume average particle diameter of the powder is preferably about 0.01 to 10 μm, and more preferably about 0.3 to 3 μm. Generally, the particle size has a distribution, but the distribution is preferably narrow.

(Phosphor Layer)

Preferred examples of the stimulable phosphor used for the phosphor layer include a stimulable phosphor represented by the formula (M_{1-f}.M_{f}^{I})X.bM^{III}X₃″:cA (formula (I)) described in JP-A No. 7-84588. From the standpoint of stimulable luminescent brightness, M^{I} in the formula (I) is preferably Rb, Cs, and/or Cs-containing Na or Cs-containing K, and particularly preferably at least one of alkali metals selected from Rb and Cs. M^{III} is preferably at least one of trivalent metals selected from Y, La, Lu, Al, Ga, and In. X″ is preferably at least one of halogens selected from F, Cl, and Br. The b value expressing the rate of content of M^{III}X₃″ is preferably selected from a range of 0<b<10⁻².

In the formula (I), the activator A is preferably at least one of metal selected from Eu, Tb, Ce, Tm, Dy, Ho, Gd, Sm, Tl, and Na, and particularly preferably at least one of metal selected from Eu, Ce, Sm, Tl, and Na. Moreover, the C value expressing the amount of activator is preferably selected from a range of 10⁻⁶<C<0.1, from the point of stimulable luminescent brightness.

Moreover, the following stimulable phosphors may be used: SrS:Ce, Sm, SrS:Eu, Sm, ThO₂:Er, and La₂O₂S:Eu, and Sm, described in U.S. Pat. No. 3,859,527;

ZnS:Cu, Pb, BaO.xAl₂O₃:Eu (wherein 0.8<x<10), and M^{II}O.xSiO₂:A (wherein: M^{II} is Mg, Ca, Sr, Zn, Cd, or Ba; A is Ce, Tb, Eu, Tm, Pb, Tl, Bi, or Mn; and x is 0.5<x<2.5) described in JP-AJP-A No. 55-12142;

(Ba_{1-x-y}, MgX, Cay) FX:aEu²⁺ (wherein: X is at least one of Cl and Br; x and y is 0<x+y<0.6; and xy≠0, and a is 10⁻⁶<a<5×10⁻²) described in JP-A No. S55-12143;

LnOX:xA (wherein: Ln is at least one of La, Y, Gd, and Lu; X is at least one of Cl and Br; A is at least one of Ce and Tb; and x is 0<x<0.1) described in JP-AJP-A No. 55-12144;

(Ba₁₋ₓ, M²⁺X) FX:yA (wherein: M is at least one of Mg, Ca, Sr, Zn, and Cd; X is at least one of Cl, Br, and I; A is at least one of Eu, Tb, Ce, Tm, Dy, Pr, Ho, Nd, Yb, and Er; x is 0<x<0.6; and y is 0<y<0.2) described in JP-AJP-A No. 55-12145;

phosphors represented by the composition formula of M^{II}FX.xA:yLn (wherein: M^{II} is at least one of Ba, Ca, Sr, Mg, Zn, and Cd; A is at least one of BeO, MgO, CaO, SrO, BaO, ZnO, Al₂O₃, Y₂O₃, La₂O₃, In₂O₃, SiO₂, TiO₂, ZrO₂, GeO₂, SnO₂, Nb₂O₅, Ta₂O₅, and ThO₂; Ln is at least one of Eu, Tb, Ce, Tm, Dy, Pr, Ho, Nd, Yb, Er, Sm, and Gd; X is at least one of Cl, Br, and I; and x and y are respectively 5×10⁻⁵<x<0.5 and 0<y<0.2) described in JP-AJP-A No. 55-160078;

phosphors represented by the composition formula of (Ba₁₋ₓ, M^{II}ₓ) F₂.aBaX₂:yEu, zA (wherein: M^{II} is at least one of beryllium, magnesium, calcium, strontium, zinc, and cadmium; X is at least one of chlorine, bromine, and iodine; A is at least one of zirconium and scandium; and a, x, y, and z are respectively 0.5<a<1.25, 0<x<1, 10⁻⁶<y<2×10⁻¹, and 0<z<10⁻²) described in JP-AJP-A No. 56-116777;

phosphors represented by the composition formula of (Ba₁₋ₓ, M^{II}ₓ)F₂.aBaX₂:yEu, zB (wherein: M^{II} is at least one of beryllium, magnesium, calcium, strontium, zinc, and cadmium; X is at least one of chlorine, bromine, and iodine; and a, x, y, and z are respectively 0.5<a<1.25, 0<x<1, 10⁻⁶<y<2×10⁻¹, and 0<z<10⁻²) described in JP-A No. S57-23673;

phosphors represented by the composition formula of (Ba₁₋ₓ, M^{II}ₓ)F₂.aBaX₂:yEu, zA (wherein: M^{II} is at least one of beryllium, magnesium, calcium, strontium, zinc, and cadmium; X is at least one of chlorine, bromine, and iodine; A is at least one of arsenic and silicon; and a, x, y, and z are respectively 0.5<a<1.25, 0<x<1, 10⁻⁶<y<2×10⁻¹, and 0<z<5×10⁻¹) described in JP-A No. 57-23675;

phosphors represented by the composition formula of M^{III}OX:xCe (wherein: M^{III} is at least one of trivalent metal selected from a group consisting of Pr, Nd, Pm, Sm, Eu, Tb, Dy, Ho, Er, Tm, Yb, and Bi; X is either Cl or Br, or both of them; and x is 0<x<0.1) described in JP-A No. 58-69281;

phosphors represented by the composition formula of Ba₁₋ₓM_{x/2}F_{x/2}Fₓ:yEu²⁺ (wherein: M represents at least one of alkali metal selected from a group consisting of Li, Na, K, Rb, and Cs; L represents at least one of trivalent metal selected from a group consisting of Sc, Y, La, Ce, Pr, Nd, Pm, Sm, Gd, Tb, Dy, Ho, Er, Tm, Yb, Lu, Al, Ga, In, and Tl; X represents at least one of halogen selected from a group consisting of Cl, Br and I; x is 10⁻²<x<0.5; and y is 0<y<0.1) described in JP-A No. 58-206678;

phosphors represented by the composition formula of BaFX.xA:yEu²⁺ (wherein: X is at least one of halogen selected from a group consisting of Cl, Br, and I; A is a burned product of tetrafluoroborate compound; and x is 10⁻⁶<x<0.1, and y is 0<y<0.1) described in JP-A No. 59-27980;

phosphors represented by the composition formula of BaFX.xA:yEu²⁺ (wherein: X is at least one of halogen selected from a group consisting of Cl, Br, and I; A is a burned product of at least one of compound selected from a hexafluoro compound group consisting of monovalent or divalent metal salt of hexafluorosilicic acid, hexafluorotitanic acid, and hexafluorozirconic acid; x is 10⁻⁶<x<0.1; and y is 0<y<0.1) described in JP-A No. 59-47289;

phosphors represented by the composition formula of BaFX.xNaX′:aEu²⁺ (wherein: X and X′ are respectively at least one of Cl, Br, and I; and x and a are respectively 0<x<2 and 0<a<0.2) described in JP-A No. 59-56479;

phosphors represented by the composition formula of M^{II}FX.xNaX′:yEu²⁺:zA (wherein: M^{II} is at least one of alkaline earth metal selected from a group consisting of Ba, Sr, and Ca; X and X′ are respectively at least one of halogen selected from a group consisting of Cl, Br, and I; A is at least one of transition metal selected from V, Cr, Mn, Fe, Co, and Ni; x is 0<x<2, y is 0<y<0.2; and z is 0<z<10⁻²) described in JP-A No. 59-56480;

phosphors represented by the composition formula of M^{II}FX.aM^{I}X′.bM′^{II}X″₂.cM^{III}X₃.xA:yEu²⁺ (wherein: M^{II} is at least one of alkaline earth metal selected from a group consisting of Ba, Sr, and Ca; M^{I} is at least one of alkali metal selected from a group consisting of Li, Na, K, Rb, and Cs; M′^{I} is at least one of divalent metal selected from a group consisting of Be and Mg; M^{III} is at least one of trivalent metal selected from a group consisting of Al, Ga, In, and Tl; A is a metal oxide; X is at least one of halogen selected from a group consisting of Cl, Br, and I; X′, X″, and X are at least one of halogen selected from a group consisting of F, Cl, Br, and I; a is 0<a<2, b is 0<b<10⁻², c is 0<c<10⁻², and a+b+c>10⁻⁶; x is 0<x<0.5; and y is 0<y<0.2) described in JP-A No. 59-75200;

stimulable phosphors represented by the composition formula of M^{II}X₂.aM^{II}X′₂:xEu²⁺ (wherein M^{II} is at least one of alkaline earth metal selected from a group consisting of Ba, Sr, and Ca; X and X′ are at least one of halogen selected from a group consisting of Cl, Br, and I, and X≠X′; a is 0.1<a<10.0; and x is 0<x<0.2) described in JP-A No. 60-84381;

stimulable phosphors represented by the composition formula of M^{II}FX.aM^{I}X′:xEu²⁺ (wherein: M^{II} is at least one of alkaline earth metal selected from a group consisting of Ba, Sr, and Ca; M^{I} is at least one of alkali metal selected from a group consisting of Rb and Cs; X is at least one of halogen selected from a group consisting of Cl, Br, and I; X′ is at least one of halogen selected from a group consisting of F, Cl, Br, and I; and a and x are respectively 0<a<4.0 and 0<x<0.2) described in JP-A No. 60-101173;

stimulable phosphors represented by the composition formula of M^{I}X:xBi (wherein: M^{I} is at least one of alkali metal selected from a group consisting of Rb and Cs; X is at least one of halogen selected from a group consisting of Cl, Br, and I; and x is a numerical value within a range of 0<x<0.2) described in JP-A No. 62-25189; and

cerium-activated rare earth oxyhalide phosphors represented by LnOX:xCe (wherein: Ln is at least one of La, Y, Gd, and Lu; X is at least one of Cl, Br, and I; x is 0<x<0.2; the ratio of X to Ln is 0.500<X/Ln<0.998 in atom ratio; and the maximum wavelength λ of the stimulable exciton spectrum is 550 nm<λ<700 nm) described in JP-A No. 2-229882.

Moreover, M^{II}X₂.aM^{II}X′₂:xEu²⁺ stimulable phosphors described in the JP-A No. 60-84381 may contain additives as shown below.

That is, bM^{I}X″ (wherein: M^{I} is at least one of alkali metal selected from a group consisting of Rb and Cs; X″ is at least one of halogen selected from a group consisting of F, Cl, Br, and I; and b is 0<b<10.0) described in JP-A No. 60-166379; bKX″.cMgX₂.dM^{III}X′₃ (wherein: M^{III} is at least one of trivalent metal selected from a group consisting of Sc, Y, La, Gd, and Lu; X″, X, and X′ are all at least one of halogen selected from a group consisting of F, Cl, Br, and I; and b, c, and d are respectively 0<b<2.0, 0<c<2.0, 0<d<2.0, and 2×10⁻⁵<b+c+d) described in JP-A No. 60-221483; yB (wherein y is 2×10⁻⁴<y<2×10⁻¹) described in JP-A No. 60-228592; bA (wherein: A is at least one of oxide selected from a group consisting of SiO₂ and P₂O₅; and b is 10⁻⁴<b<2×10⁻¹) described in JP-A No. 60-228593; bSiO (wherein b is 0<b<3×10⁻²) described in JP-A No. 61-120883; bSnX″₂ (wherein: X″ is at least one of halogen selected from a group consisting of F, Cl, Br, and I; and b is 0<b<10⁻³) described in JP-A No. 61-120885; bCsX″.cSnX₂ (wherein: X″ and X are respectively at least one of halogen selected from a group consisting of F, Cl, Br, and I; and b and c are respectively 0<b<10.0 and 10⁻⁶<c<2×10⁻²) described in JP-A No. 61-235486; and bCsX″.yLn³⁺ (wherein: X″ is at least one of halogen selected from a group consisting of F, Cl, Br, and I; Ln is at least one of rare earth selected from a group consisting of Sc, Y, Ce, Pr, Nd, Sm, Gd, Tb, Dy, Ho, Er, Tm, Yb, and Lu; and b and y are respectively 0<b<10.0 and 10⁻⁶<y<1.8×10⁻¹) described in JP-A No. 61-235487.

Among the above stimulable phosphors, divalent europium-activated alkaline earth metal fluorohalide phosphors (such as BaFI:Eu), europium-activated alkali metal halide phosphors (such as CsBr:Eu), iodine-containing divalent europium-activated alkaline earth metal halide phosphors, iodine-containing rare earth element-activated rare earth oxyhalide phosphors, and iodine-containing bismuth-activated alkali metal halide phosphors can be preferably used since they show a high stimulable luminescent brightness.

(Protective Layer)

For the protective layer formed on the phosphor layer, there may be used: a layer formed such that a solution that has been prepared by dissolving a transparent organic high molecular material such as cellulose derivative and polymethyl methacrylate in an appropriate solvent, is coated on the phosphor layer; a sheet for forming a protective film such as a transparent glass plate or an organic high molecular film of polyethylene terephthalate and the like that is separately formed, and provided on the surface of the phosphor layer using an appropriate adhesive; or a film of an inorganic compound formed on the phosphor layer by means of deposition or the like.

Moreover, it may be a protective layer formed from a coated film of an organic solvent-soluble fluororesin, having fine particles such as perfluoroolefine resin powder, silicone resin powder, and TiO₂ particles dispersed and contained therein.

As described above, in order to keep the thermal shrinkage rate (JISC2151, at 150° C. for 30 minutes) of the protective layer 1% or less, there is preferably employed a material that has been previously treated by heat annealing, having a high Tg (glass transition temperature: JIS K7121 (1987)). Moreover, preferably a heat treatment of 60° C. or more, is applied at least either before or at the time of its formation.

Hereunder, exemplary aspects of the present invention are enumerated. <1> An image reading device, comprising: a disinfection unit that administers a disinfection treatment to an imaging medium carrying a radiation image or to a protective member covering at least an imaging surface of the imaging medium; and an image reading unit that reads the radiation image carried by the imaging medium either after or before the disinfection treatment by the disinfection unit.

According to the image reading device recited in <1>, uniform and effective disinfection treatment can be implemented with respect to an imaging medium having a radiation image that is read by an image reading unit and a protective member that covers at least an imaging surface of the imaging medium.

<2> The image reading device recited in <1>, wherein the disinfection treatment is at least one treatment selected from the group consisting of heat treatment, ultraviolet ray irradiation treatment, chemical coating treatment and gas treatment.

<3> The image reading device recited in <2>, wherein: the imaging medium is a radiation image conversion panel; and the disinfection treatment by the disinfection unit is heat treatment, and the heat treatment comprises heating the radiation image conversion panel at 60° C. to 200° C. for 1 second to 10 minutes.

<4> The image reading device according to any one of <1> to <3>, wherein the imaging medium is a radiation image conversion panel having a protective layer with a thermal shrinkage rate of 1% or less at 150° C. for 30 minutes.

<5> The image reading device recited in <4>, wherein the protective layer of the radiation image conversion panel is subjected to heat treatment at 60° C. or above at either or both of before and during formation thereof.

<6> The image reading device according to any one of <2> to <5>, wherein the disinfection treatment by the disinfection unit is heat treatment and the disinfection unit is equipped with a temperature control unit.

<7> The image reading device according to any one of <2> to <6>, wherein the disinfection treatment by the disinfection unit is heat treatment and the heat treatment comprises heating with either or both of an infrared heater and a far-infrared heater.

<8> The image reading device recited in <2>, wherein the disinfection treatment by the disinfection unit is ultraviolet ray irradiation treatment, and irradiation energy of ultraviolet rays in the ultraviolet ray irradiation treatment is 0.04 J/cm² or above.

According to the image reading device recited in <2> to <8>, similarly to <1>, uniform and effective disinfection treatment can be implemented with respect to an imaging medium having a radiation image read by an image reading unit and a protective member that covers at least an imaging surface of the imaging medium.

<9> The image reading device according to any one of <1> to <8>, further comprising: an insertion port through which the imaging medium is inserted; a conveying unit that conveys the imaging medium that has been inserted through the insertion port; a residual image erasing unit that erases from the imaging medium a residual image of the radiation image carried by the imaging medium after the radiation image has been read by the image reading unit; and a discharge port through which the imaging medium is discharged after the residual image is erased by the residual image erasing unit, wherein: the disinfection unit disinfects the imaging medium that has been inserted through the insertion port; and the image reading unit reads the radiation image carried by the imaging medium from the imaging medium that has been disinfected by the disinfection unit.

In the image reading device recited in <9>, an imaging medium carrying a radiation image is inserted through an insertion port and conveyed by a conveyance unit. The imaging medium is first disinfected by a disinfection unit, then the radiation image is imaged by an image reading unit and, after the residual image of the radiation image is then erased by a residual image erasing unit, the imaging medium is discharged from a discharge port.

As a result, it is possible to make a region inside the image reading device at a downstream side of the disinfection unit in the direction of conveyance, a clean region through which the imaging medium passes after having been disinfected. Further, disinfection of the imaging medium by an operator prior to inserting the imaging medium into the image reading device is unnecessary. Accordingly, it is possible to both suppress the propagation of bacteria inside the image reading device and reduce the workload an operator.

<10> The image reading device recited in <9>, wherein the discharge port is separated from the insertion port.

In the image reading device recited in <10>, it is possible to discharge the disinfected imaging medium to the outside of the device such that it is not made to pass the disinfection unit a second time, by making the discharge port separated from the insertion port. Consequently, adhesion of bacteria to the discharged imaging medium can be suppressed.

<11> The image reading device recited in <9> or <10>, further comprising a device housing that accommodates at least the image reading unit and the residual image erasing unit and that the disinfection unit is freely attachable to and detachable from.

In the image reading device recited in <11>, the disinfection unit is freely attached to and detached from the device housing accommodating the image reading unit and the residual image erasing unit. As a result, it is possible to add an imaging medium disinfection function to a conventional image reading device that is not equipped with a disinfection unit.

<12> The image reading device according to any one of <9> to <11>, further comprising a protective member removal unit that is disposed at a downstream side of the insertion port in a direction of conveyance and at an upstream side of the disinfection unit in the direction of conveyance, and that removes the protective member from the imaging medium, wherein the insertion port is configured such that the protective member can be inserted together with the imaging medium.

In the image reading device recited in <12>, a protective member that covers at least an imaging surface of an imaging medium is inserted via an insertion port together with the imaging medium, and is removed from the imaging medium by a protective member removal unit. As a result, the workload of an operator can be reduced because it is not necessary to manually remove the protective member from the imaging medium. Further, contamination of the imaging surface of the imaging medium can be further suppressed because the imaging medium is inserted into the image reading device in a state in which the imaging surface is protected by the protective member.

<13> The image reading device according to any one of <9> to <12>, further comprising a protective member attachment unit that is disposed at a downstream side of the residual image erasing unit in a direction of conveyance, and that attaches the protective member to the imaging medium.

In the image reading device recited in <13>, after the radiation image carried by the imaging medium is erased by the residual image erasing unit, a protective member is attached to the imaging medium by a protective member attachment unit and the imaging surface of the imaging medium is covered by the protective member.

As s result, the workload of an operator can be reduced because it is not necessary to manually attach the protective member to an imaging medium that has been discharged from the image reading device. Further, contamination of the imaging surface of the imaging medium can be further suppressed because the imaging medium is discharged from the image reading device in a state in which the imaging surface is protected by the protective member.

<14> The image reading device recited in <13>, further comprising a pack enclosure unit that is disposed at a downstream side of the protective member attachment unit in the direction of conveyance, and that encloses the imaging medium within a contamination-prevention pack that prevents adhesion of contaminants to the imaging medium.

In the image reading device recited in <14>, the imaging medium, which has had a protective member attached thereto by the protective member attachment unit, is enclosed within a contamination-prevention pack by a pack enclosure unit.

As a result, the workload of an operator can be reduced because it is not necessary to manually enclose within a contamination-prevention pack an imaging medium that has been discharged from the image reading device. Further, contamination of not only the imaging medium, but also of the protective member, can be suppressed because the imaging medium, which has had a protective member attached thereto, is discharged from the image reading device in a state in which it is enclosed within a contamination-prevention pack.

<15> The image reading device according to any one of <9> to <14>, further comprising: a partition member that partitions the inside of the device into a disinfection chamber accommodating the disinfection unit and an image processing chamber accommodating the image reading unit; and a chamber pressure maintenance unit that maintains the chamber pressure of the image processing chamber at a higher pressure than the chamber pressure of the disinfection chamber.

In the image reading device recited in <15>, the inside of the device is partitioned into a disinfection chamber accommodating the disinfection unit and an image processing chamber accommodating the image reading unit by a partition member. The chamber pressure of the image processing chamber is maintained at a higher pressure than the chamber pressure of the disinfection chamber by a chamber pressure maintenance unit.

As a result, entry of bacteria into the image processing chamber from inside the disinfection chamber can be suppressed and proliferation of bacteria at the image processing chamber can be suppressed.

<16> The image reading device according to any one of <1> to <8>, further comprising: an insertion port through which the imaging medium is inserted; a conveying unit that conveys the imaging medium that has been inserted through the insertion port; a residual image erasing unit that is disposed at a downstream side of the image reading unit in a direction of conveyance and that erases a residual image of the radiation image carried by the imaging medium; and a discharge port through which the imaging medium is discharged, that is disposed at a downstream side of the residual image erasing unit and the disinfection unit in the direction of conveyance, and that is different from the insertion port, wherein: the image reading unit is disposed at a downstream side of the insertion port in the direction of conveyance; and the disinfection unit is disposed at a downstream side of the image reading unit in the direction of conveyance.

In the image reading device recited in <16>, an imaging medium carrying a radiation image is inserted through an insertion port and conveyed by a conveying unit. The radiation image is first read by an image reading unit, then a residual image of the radiation image is erased by a residual image erasing unit and, after the imaging medium is disinfected by a disinfection unit, the imaging medium is discharged from a discharge port that is different from the insertion port.

That is, it is possible to suppress lengthening of the time required from insertion of the imaging medium into the image reading device until reading of the radiation image because reading of the radiation image by the image reading unit is performed prior to disinfection of the imaging medium by the disinfection unit. Further, the workload of an operator can be reduced because it is not necessary for disinfection of the imaging medium to be performed by the operator.

<17> The image reading device recited in <16>, wherein the disinfection treatment by the disinfection unit is performed during residual image erasing processing by the residual image erasing unit.

In the image reading device recited in <17>, the time required until the imaging medium is discharged can be shortened because disinfection treatment is performed by the disinfection unit during erasing of the residual image by the residual image erasing unit.

<18> The image reading device recited in <16> or <17>, wherein the residual image erasing unit is integrated with the disinfection unit.

In the image reading device recited in <18>, the space occupied by the residual image erasing unit and the disinfection unit can be reduced by integration of the residual image erasing unit and the disinfection unit, and the size of the image reading device can be reduced.

<19> The image reading device according to any one of <16> to <18>, further comprising a device housing that accommodates at least the image reading unit and that the disinfection unit is freely attachable to and detachable from.

In the image reading device recited in <19>, the disinfection unit is freely attached to and detached from the device housing accommodating the image reading unit. As a result, it is possible to add an imaging medium disinfection function to a conventional image reading device that is not equipped with a disinfection unit.

<20> The image reading device according to any one of <16> to <19>, further comprising a protective member removal unit that is disposed at a downstream side of the insertion port in the direction of conveyance and at an upstream side of the image reading unit in the direction of conveyance, and that removes the protective member from the imaging medium, wherein the insertion port is configured such that the protective member can be inserted together with the imaging medium.

In the image reading device recited in <20>, a protective member that covers at least the imaging surface of the imaging medium is inserted through the insertion port together with the imaging medium and is removed from the imaging medium by the protective member removal unit. As a result, As a result, the workload of an operator can be reduced because it is not necessary to manually remove the protective member from the imaging medium. Further, contamination of the imaging surface of the imaging medium can be further suppressed because the imaging medium is inserted into the image reading device in a state in which the imaging surface is protected by the protective member.

<21> The image reading device according to any one of <16> to <20>, further comprising a protective member attachment unit that is disposed at a downstream side of the residual image erasing unit and the disinfection unit in the direction of conveyance, and that attaches the protective member to the imaging medium.

In the image reading device recited in <21>, after the radiation image is erased from the imaging medium by the residual image erasing unit and the imaging medium is disinfected by the disinfection unit, a protective member is attached by a protective member attachment unit and the imaging surface of the imaging medium is covered by the protective member.

As a result, the workload of an operator can be reduced because it is not necessary to manually attach the protective member to an imaging medium that has been discharged from the image reading device. Further, contamination of the imaging surface of the imaging medium can be suppressed because the imaging medium is discharged from the image reading device in a state in which the imaging surface is protected by the protective member.

<22> The image reading device recited in <21>, further comprising a pack enclosure unit that is disposed at a downstream side of the protective member attachment unit in the direction of conveyance, and that encloses the imaging medium within a contamination-prevention pack that prevents adhesion of contaminants to the imaging medium.

In the image reading device recited in <22>, the imaging medium, which has had a protective member attached thereto by the protective member attachment unit, is enclosed within a contamination-prevention pack by a pack enclosure unit.

As a result, the workload of an operator can be reduced because it is not necessary to manually enclose within a contamination-prevention pack an imaging medium that has been discharged from the image reading device. Further, contamination of not only the imaging medium, but also of the protective member, can be suppressed because the imaging medium, which has had a protective member attached thereto, is discharged from the image reading device in a state in which it is enclosed within a contamination-prevention pack.

<23> The image reading device according to any one of <1> to <8>, further comprising: an insertion port through which the imaging medium is inserted;

a conveying unit that conveys the imaging medium that has been inserted through the insertion port; a cleaning unit that cleans the imaging medium that has been inserted through the insertion port; a residual image erasing unit that erases from the imaging medium a residual image of the radiation image carried by the imaging medium after the radiation image has been read by the image reading unit; and a discharge port through which the imaging medium is discharged after the residual image is erased by the residual image erasing unit, wherein: the image reading unit reads the radiation image carried by the imaging medium from the imaging medium that has been disinfected by the cleaning unit.

In the image reading device recited in <23>, an image medium carrying a radiation image is inserted through an insertion port and is conveyed by a conveying unit. The imaging medium is first cleaned by a cleaning unit, then the radiation image is read by an image reading unit and, then, a residual image of the radiation image is erased by a residual image erasing unit.

As a result, it is possible to have the radiation image read by the image reading unit from a cleaned imaging medium. Further, it is not necessary for an operator to clean the imaging medium before insertion into the image reading device. Consequently, reduction in the reading performance of the radiation image carried by the imaging medium can be suppressed and the workload of an operator can be reduced.

<24> The image reading device recited in <23>, further comprising a protective member removal unit that removes the protective member from the imaging unit after the imaging medium has been inserted through the insertion port and before the imaging medium has been cleaned by the cleaning unit, wherein the insertion port is configured such that the protective member can be inserted together with the imaging medium.

In the image reading device recited in <24>, a protective member that covers at least the imaging surface of an imaging medium is inserted through an insertion port together with the imaging medium and is removed from the imaging medium by a protective member removal unit. As a result, the workload of an operator can be reduced because it is not necessary to manually remove the protective member from the imaging medium. Further, contamination of the imaging surface of the imaging device can be further suppressed because the imaging medium can be inserted into the image reading device in a state in which the imaging surface is protected by the protective member.

<25> The image reading device according to any one of <1> to <8>, further comprising: an insertion port through which the imaging medium is inserted in a state in which at least the imaging surface is protected by the protective member; a conveying unit that conveys the imaging medium that has been inserted through the insertion port; a cleaning unit that cleans the protective member that has been inserted through the insertion port; a protective member removal unit that removes from the imaging medium the protective member that has been cleaned by the cleaning unit; and a residual image erasing unit that erases from the imaging medium a residual image of the radiation image carried by the imaging medium after the radiation image has been read by the image reading unit, wherein the image reading unit reads from the imaging medium the radiation image carried by the imaging medium after the protective member has been removed by the protective member removal unit.

In the image reading device recited in <25>, an imaging medium carrying a radiation image is inserted through an insertion port in a state in which at least the imaging surface is protected by a protective member and is conveyed by a conveyance unit. After insertion of the imaging medium, the protective member is first cleaned by a cleaning unit and, then, the protective member is removed from the imaging medium by a protective member removal unit. After this, the radiation image is read from the imaging medium by an image reading unit and, further, a residual image of the radiation image is removed by a residual image removal unit.

As a result, it is possible to suppress the adhesion of contaminants such as saliva or blood adhered to the protective member, to the imaging surface of the imaging medium when the protective member is removed from the imaging medium by the protective member removal unit. Further, cleaning of the protective member attached to the imaging medium by an operator is not necessary. Consequently, reduction in the reading performance of the radiation image carried by the imaging medium can be suppressed and the workload of an operator can be reduced.

<26> The image reading device according to any one of <23> to <25>, wherein the discharge port is separated from the insertion port.

In the image reading device recited in <26>, the cleaned imaging medium can be discharged to the outside of the device without causing it to pass the cleaning unit a second time, by making the discharge port separated from the insertion port. Consequently, adhesion of contaminants to the discharged imaging medium can be suppressed.

<27> The image reading device according to any one of <23> to <26>, further comprising a device housing that accommodates at least the image reading unit and an image removal unit, and that the cleaning unit is freely attachable to and detachable from.

In the image reading device recited in <27>, the cleaning unit is freely attached to and detached from the device housing accommodating the image reading unit and the image erasing unit. As a result, it is possible to add an imaging medium cleaning function to a conventional image reading device that is not equipped with a cleaning unit.

<28> The image reading device according to any one of <23> to <27>, wherein the disinfection unit is disposed at a downstream side of the cleaning unit in a direction of conveyance.

In the image reading device recited in <28>, the imaging medium is disinfected by the disinfection means after the imaging medium is cleaned by the cleaning unit. As a result, the workload of an operator can be reduced because disinfection of the imaging medium discharged from the image reading device by the operator is not necessary.

<29> The image reading device recited in <28>, further comprising a protective member attachment unit that is disposed at a downstream side of the disinfection unit in a direction of conveyance, and that attaches the protective member to the imaging medium.

In the image reading device recited in <29>, a protective member is attached to the imaging medium by a protective member attachment unit after the imaging unit is disinfected by a disinfection unit, and the imaging surface of the imaging unit is covered by the protective member.

As a result, the workload of an operator can be reduced because it is not necessary to attach a protective member to an imaging medium that has been discharged from the image reading device. Further, contamination of the imaging surface of the imaging medium can be further suppressed because the imaging plate is discharged from the image reading device in a state in which the imaging surface is protected by the protective member.

<30> The image reading device recited in <29>, further comprising a pack enclosure unit that is disposed at a downstream side of the protective member attachment unit in the direction of conveyance, and that encloses the imaging medium within a contamination-prevention pack that prevents adhesion of contaminants to the imaging medium.

In the image reading device recited in <30>, an imaging medium, having had a protective member attached thereto by a protective member enclosure unit, is enclosed within a contamination-prevention pack by a pack enclosure unit.

As a result, the workload of an operator can be reduced because it is not necessary to manually enclose within a contamination-prevention pack an imaging medium that has been discharged from the image reading device. Further, contamination of not only the imaging medium, but also of the protective member, can be suppressed because the imaging medium, which has had a protective member attached thereto, is discharged from the image reading device in a state in which it is enclosed within a contamination-prevention pack.

### EXAMPLES

### Example 1

### Formation of Interlayer

3400 g of soft acrylic resin (trade name: CRISCOAT P-1018GS manufactured by Dainippon Ink and Chemicals, Incorporated (21% toluene solution)) as a binder and 120 g of phthalic acid ester (trade name: #10 manufactured by Daihachi Chemical Industry Co., Ltd.) as a plasticizer were added and mixed in 3600 g of methyl ethyl ketone, and then dispersed and dissolved using a disper to prepare a dispersion solution for forming an interlayer (viscosity 0.6 Pa.s (20° C.)).

A conductive agent and a coloring agent were used which were dispersed by a ball mill in the solution to which a resin had been previously added. This dispersion solution for forming an interlayer was evenly coated on a support (carbon-kneaded polyethylene terephthalate, trade name: X-30 manufactured by Toray Industries, Inc., thickness: 188 μm) to form a coated layer, and was then dried. By so doing, an interlayer having a thickness of 20 μm was formed.

(Production of Phosphor Sheet)

A phosphor sheet to become a phosphor layer was produced as follows. Firstly, as a coating solution for forming a phosphor sheet, 1000 g of phosphor (BaFBr_{0.85}I_{0.15}:Eu²⁺, median diameter 3.5 μm), 36 g of polyurethane elastomer (trade name: PANDEX T5265H (solid)) manufactured by Dainippon Ink and Chemicals, Incorporated) serving as a binder, 4 g of polyisocyanate (trade name: CORONATE HX (solid content 100%) manufactured by Nippon Polyurethane Industry Co., Ltd.) serving as a crosslinking agent, 10 g of epoxy resin (trade name: EPICOAT 1001 (solid) manufactured by Yuka Shell Epoxy Co., Ltd.) serving as an anti-yellowing agent, and 2 g of ultramarine (trade name: SM-1 manufactured by Daiishikasei Co., Ltd.) serving as a coloring agent were added into 120 g of mixed solvent of methyl ethyl ketone and butyl acetate (methyl ethyl ketone/butyl acetate (mass ratio)=6/4), and then dispersed using a disper at a blade rotation speed of 2500 rpm for 1 hour to prepare a coating solution having a viscosity of 4.0 Pa.s (25° C.). A coloring agent was used which was dispersed by a ball mill in the solvent to which a resin had been previously added.

This coating solution was evenly coated on a temporary support (polyethylene terephthalate coated with a silicone release material, thickness: 180 μm)) and dried. Then, it was peeled off from the temporary support to produce a phosphor sheet (thickness 150 μm).

(Formation of Phosphor Layer)

Next, the face of the phosphor sheet from which the temporary support was peeled off, was superposed on the interlayer using a calendar roll by a continuous compression operation under a pressure of 60 MPa, at a roll temperature of 50° C., and at a feed speed of 1.0 m/min. By this heat compression, the phosphor sheet was completely adhered onto the support through the interlayer, and the phosphor layer was formed on the support.

(Formation of Protective Layer)

A PET film having a thickness of 6 μm and a PET film having a thickness of 50 λm were adhered to each other through a repeelable adhesive layer, then heat treated at 1001C. The PET film having a thickness of 6 μm was peeled off, and one face thereof was coated with an unsaturated polyester resin solution (trade name: VYLON 30SS manufactured by Toyobo Co., Ltd.), and then dried at 80° C. to provide an adhesive layer. The PET film was adhered onto the phosphor layer through the adhesive layer, to form a protective layer.

Next, this sheet was blanked into an appropriate size (square of 3 cm×3 cm) by a blanking blade (male blade and female blade). Then, a resin (DIAROMER SP3023: EP1004: X-22-2809: CROSSNATE D70=900:8:2:30 dissolved in MEK) was coated on the surface of the protective layer at the periphery of the blanked sheet with a width of 0.5 to 1 mm extending inward, and then dried (at 50° C.) to produce a radiation image conversion panel.

### Example 2

A protective layer was formed in the same manner as that of Example 1 except that one face of a PET film having a thickness of 9 μm was coated with an unsaturated polyester resin solution (trade name: VYLON 30SS manufactured by Toyobo Co., Ltd.), and heat treated at 80° C. to provide the adhesive layer, to produce a radiation image conversion panel.

### Example 3

A protective layer was formed in the same manner as that of Example 1 except that one face of a PET film having a thickness of 6 μm was coated with an unsaturated polyester resin solution (trade name: VYLON 30SS manufactured by Toyobo Co., Ltd.), and dried at 50° C. to provide the adhesive layer, to produce a radiation image conversion panel.

### Comparative Example 1

A protective layer was formed in the same manner as that of Example 1 except that one face of a PET film having a thickness of 9 μm was coated with an unsaturated polyester resin solution (trade name: VYLON 30SS manufactured by Toyobo Co., Ltd.), and heat treated at 80° C. to provide the adhesive layer, to produce a radiation image conversion panel.

[Measurement of Shrinkage Rate of Protective Layer]

The shrinkage rate of the protective layer on the radiation image conversion panel was measured based on JISC2151 (at 150° C. for 30 minutes). The results are shown in Table 1 below.

[Evaluation of Disinfection Treatment]

The same amount of MRSA was adhered onto each protective layer of the radiation image conversion panels of Examples 1 to 3 and Comparative Example 1. The MRSA was cultured by agar plate cultivation, and then adhered onto the protective layer of the radiation image conversion panel using a brush.

Each radiation image conversion panel was introduced into a scanner with the MRSA adhered thereto, and the radiographic image was read out. Then, while the image was being erased by photoirradiation by self conveyance, a disinfection treatment by heat treatment was performed on the surface of the radiation image conversion panel by an infrared heater (250 W) at a temperature and for a time as shown in Table 1 below. Then, the radiation image conversion panel was taken out from the disinfection apparatus (disinfection unit) by self conveyance, and the remaining MRSA adhered onto the surface of the protective layer was measured. Furthermore, the deformation state of the radiation image conversion panel was visually confirmed. These results are shown in Table 1 below.

The measurement of the surface temperature of the radiation image conversion panel at the time of disinfection treatment and heat control were performed as follows. Firstly, the surface of the radiation image conversion panel was brought into contact with a thermocouple to measure the temperature, and a radiation thermometer at that time was calibrated. Next, the radiation thermometer was covered so as to avoid exposure, and then the surface of the radiation image conversion panel was irradiated by the infrared heater (250 W). The surface temperature of the radiation image conversion panel was measured from the reading and the calibration factor of the radiation thermometer at that time. By feeding back the surface temperature of the radiation image conversion panel, the infrared heater was turned ON/OFF to control the surface temperature of the radiation image conversion panel.

TABLE 1

| | Shrinkage rate of | | | | |
|---|---|---|---|---|---|
| | protective layer on | | | | |
| | radiation image | Disinfection conditions | | | |
| | conversion panel | Temperature | Time | | Shape |
| | (%) | (° C.) | (seconds) | MRSA | deformation |
| Example 1 | 0.3 | 120 | 5 | Killed | None |
| Example 2 | 0.7 | 90 | 10 | Killed | None |
| Example 3 | 1.5 | 110 | 10 | Killed | Slightly |
| | | | | | deformed |
| Comparative | 0.5 | 25 | 10 | No | None |
| Example 1 | | | | change | |

According to Table 1, MRSA remained in the radiation image conversion panel of Comparative Example 1 on which no disinfection treatment by heat treatment was performed. On the other hand, in the radiation image conversion panels of Examples 1 to 3, the MRSA were killed, and no shape deformation of a degree that would be a practical problem was observed. In particular, in the cases of Example 1 and Example 2 where the heat treatment was applied before the protective layer was formed, no shape deformation was observed at all.

### Example 4

A radiation image conversion panel Example 4 was made in the same way as Example 1. The radiation image was read out in the state of having MRSA applied to the light shielding bag. After this it was introduced into the device illustrated in FIG. 3, and after reading out of the radiation image, disinfection treatment was carried out using the heater 199. Then, after the erasing processing had been carried out of the radiation image information by the erasing unit 39, the amount of MRSA, remaining on the surface of the radiation image conversion panel was investigated. Further, the condition of deformation of the radiation image conversion panel was checked by visual inspection. The result was that the remaining MRSA and condition of deformation were both found to be of the same good condition seen in Example 1.

### Example 5

Example 5 was the same as Example 4, except in that disinfection treatment and erasing processing of the radiation image information by the erasing unit 39 was carried out at one time on the radiation image conversion panel (with MRSA applied thereto) that had been loaded in the device of FIG. 4. The remaining MRSA on the surface of the radiation image conversion panel and condition of deformation of the radiation image conversion panel were checked. The result obtained was that both were found to be of the same good condition seen in Example 1.

### Example 6

The Example 6 was the same as Example 4, except in that disinfection treatment using a heater 199 was carried out in the device of FIG. 5 after erasing processing of the radiation image information by the erasing unit 39. The remaining MRSA on the surface of the radiation image conversion panel and condition of deformation of the radiation image conversion panel were checked. The result obtained was that both were found to be of the same good condition seen in Example 1.

Hereinafter, image reading devices according to the second to eighth embodiments will be described.

### SECOND EMBODIMENT

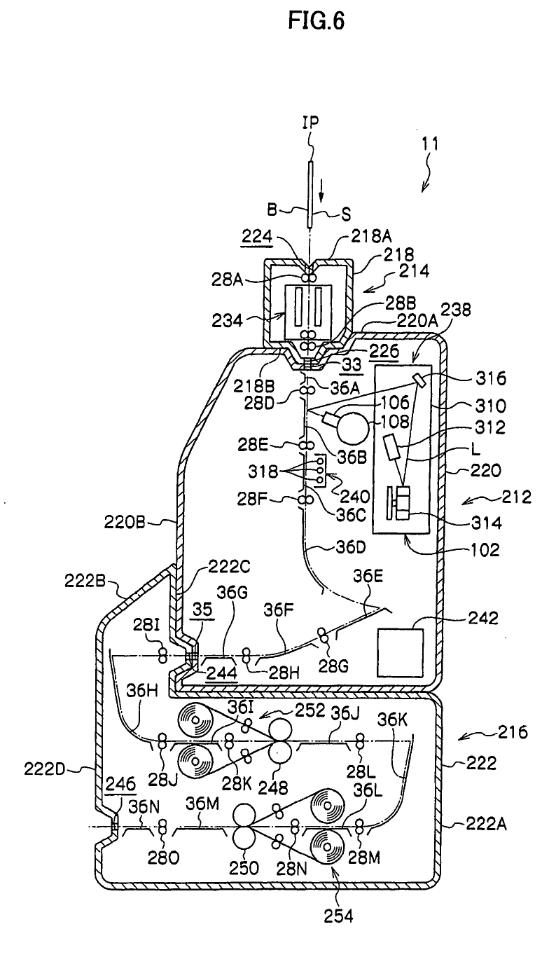

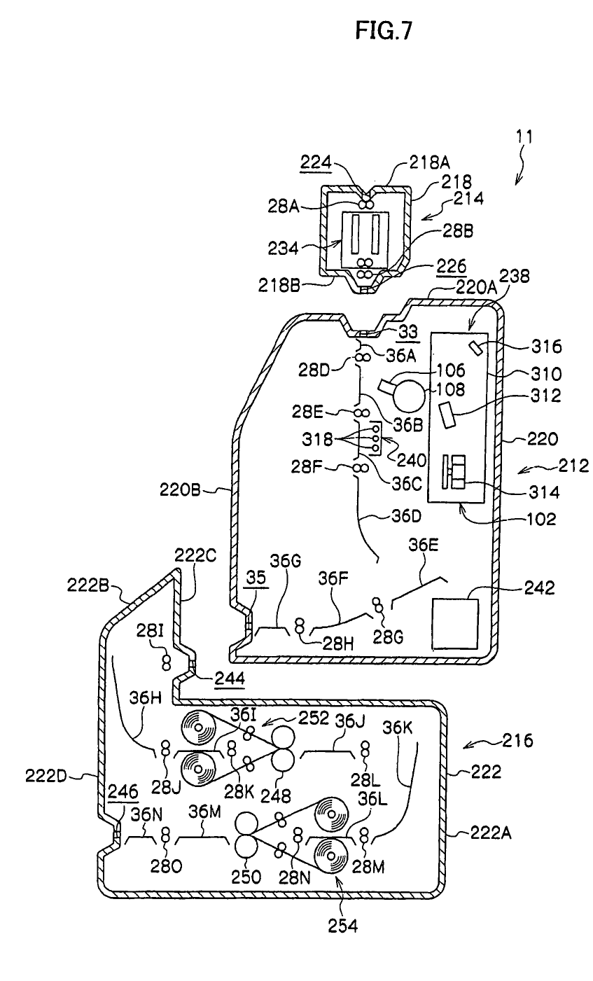

Sectional side views of schematic configurations of image reading device 11 according to the second embodiment are shown in FIGS. 6 and 7. An image to be read by image reading device 11 is an X-ray (radiation) image from an oral cavity. The image is carried on an imaging surface S of rectangular photographing plate (imaging plate) IP, which is an imaging medium that is inserted into the oral cavity.

Imaging plate IP is a plate having a photostimulable phosphor layer which stores a part of radiated X-ray energy and then exhibits photo-stimulated luminescence in response to the stored energy in response to irradiation with excitation light such as a laser beam. When imaging plate IP is irradiated with an erasing light including light in a range of excitation light wavelengths of the phosphor, the residual energy in the phosphor is erased, and imaging plate IP can be reused.

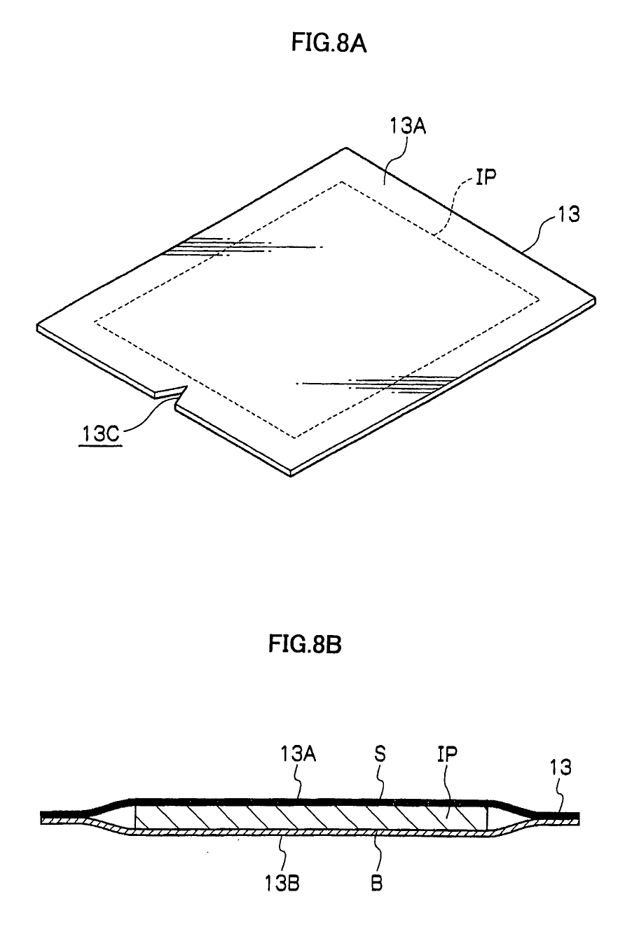

Imaging plate IP is inserted into an oral cavity in a state shown in FIGS. 8A and 8B in which imaging plate IP is enclosed in protective case 13, which is a protective member. Protective case 13 is formed by joining the peripheral portions of rectangular sheet members 13 A and 13B with each other. Sheet member 13A is made from a light-proof and water-proof material which is X-ray transmittable. The insertion direction of imaging plate IP into protective case 13 is set such that imaging surface S, which is the photostimulable phosphor layer of imaging plate IP, is covered with the sheet member 13A.

V-shaped notch 13C is formed at the central portion of one side of protective case 13, and the insertion direction of imaging plate IP into image reading device 11 is set such that this one side is the leading end. Further, the breakage strength of the protective case 13 is set to degree such that when an operator pulls both sides of the notch 13C to separate them from each other, the case is broken.

As shown in FIGS. 6 and 7, image reading device 11 is provided with image processing section 212, image pre-processing section 214, and image post-processing section 216. Image processing section 212 is housed in housing 220, image pre-processing section 214 is housed in housing 218, and image post-processing section 216 is housed in housing 222. Housing 218 and housing 220 are detachably connected to each other, and housing 220 housing 222 are detachably connected to each other, so that image processing section 212, image pre-processing section 214 and image post-processing section 216 are integrated.

Housings 218, 220 and 222 are disposed in this order from the top of the device. Housing 218 has a rectangular shape in side view. Insertion port 224 into which imaging plate 1P is inserted is provided at upper wall 218A, and discharge port 226 from which imaging plate IP is discharged is provided at lower wall 218B. In housing 218, conveying roller pairs 28A and 28B, which are conveying units, are disposed from insertion port 224 to discharge port 226, and imaging plate IP inserted from insertion port 224 into housing 218 is conveyed by conveying roller pairs 28A and 28B toward the bottom of the device to be discharged from discharge port 226.

Imaging plate IP is inserted from insertion port 224 into housing 218 in a state in which imaging surface S (see FIGS. 8A and 8B) faces the rear side of the device.

Further, in housing 218, disinfection mechanism 234, which is a disinfection unit, is disposed between conveying roller pair 28A and conveying roller pair 28B. Imaging plate IP is sterilized and disinfected by disinfection mechanism 234.

Furthermore, housing 220 is a rectangular housing in side view, and has insertion port 33 at upper wall 220A which is detachably connected to discharge port 226, and has discharge port 35 at a lower portion of front wall 220B which is a sidewall of the front side of the device. Within housing 220, conveying roller pairs 28D, 28E, 28F, 28G and 28H, and conveying guides 36A, 36B, 36C, 36D, 36E, 36F and 36G are disposed from insertion port 33 to discharge port 35 in this order, respectively.

Conveying guides 36A, 36B, 36C and 36D are disposed from insertion port 33 in this order downward in the device. Further, conveying roller pairs 28D, 28E and 28F are disposed between conveying guide 36A and conveying guide 36B, between conveying guide 36B and conveying guide 36C, and between conveying guide 36C and conveying guide 36D, respectively.

Here, conveying guide 36D is curved toward the rear side of the device, and guides imaging plate IP to the rear lower side of the device, and conveying guides 36E, 36F and 36G are disposed in this order from the lower side of conveying guide 36D at the rear of the device toward discharge port 35. Conveying guides 36E and 36F are disposed from the rear side of the device to the front side of the device, being inclined toward the lower side of the device, and conveying guide 36G is disposed substantially horizontally. Further, conveying roller pairs 28G and 28H are disposed between conveying guide 36E and conveying guide 36F, and between conveying guide 36F and conveying guide 36G, respectively.

Namely, after imaging plate IP inserted from insertion port 33 into housing 220 is conveyed by conveying roller pairs 28D, 28E and 28F to the lower part of the device, while being guided by conveying guides 36A, 36B and 36C, imaging plate IP is guided to the rear lower side of the device by conveying guide 36D, and is dropped onto conveying guide 36E.

Here, imaging plate IP is dropped onto conveying guide 36E in a state in which imaging plate IP is inclined to the rear side of the device, so that the leading end of imaging plate IP and the tail end thereof are reversed. Thus, imaging surface S dropped onto conveying guide 36E faces upward. Thereafter, imaging plate IP is guided to the front lower side of the device by conveying guide 36E inclined downward toward the front side of the device, and conveyed by conveying roller pairs 28G and 28H to the front side of the device, while being guided by conveying guides 36F and 36G, and discharged from discharge port 35.

In addition, image reading mechanism 238, which is an image reading unit, and residual image erasing mechanism 240 which is a residual image erasing unit, are arranged in this order from the upstream side in the conveying direction, and further, chamber pressure control mechanism 242, which is a chamber pressure control unit, is provided.

Image reading mechanism 238 reads an X-ray image carried on imaging surface S of imaging plate IP, and outputs image information to a monitor display (not shown). The monitor display displays an image based on the image information outputted from image reading mechanism 238. Residual image erasing mechanism 240 erases the X-ray image carried on imaging surface S of imaging plate IP. Chamber pressure control mechanism 242 blows air into housing 220 by a fan (illustrated) or the like to control the chamber pressure within housing 220 at a predetermined value which is a higher pressure than atmospheric pressure.

Housing 222 is an L-shaped housing in side view, and comprises rectangular base portion 222A in side view on which housing 220 is placed, and has front portion 222B which is provided to stand upright from the front side of base portion 222A and is detachably connected with the lower part of front wall 220B of housing 220. Insertion port 224, which is detachably connected to discharge port 35, is disposed at opposing surface 222C against the front wall 220B of front portion 222B, and discharge port 246 is disposed at front wall 222D, which is the device front side surface of base portion 222A.

Further, within housing 222, as conveying units from insertion port 224 to discharge port 246, conveying roller pairs 281, 28J and 28K, heat roller pair 248, conveying roller pairs 28L, 28M and 29N, pressure roller pair 250, conveying roller pair 280, conveying guides 36H, 36I, 36J, 36K, 36L, 36M and 36N are arranged in this order, respectively. Conveying roller pair 28I conveys imaging plate IP inserted from insertion port 244 to the front side of the device.

Conveying guide 36H is disposed at the front side of conveying roller pair 28I. Conveying guide 36H is inclined downward from the front side toward the rear side of the device, and imaging plate IP is guided (dropped) to the front lower side of the device with the leading end and the tail end of imaging plate IP reversed, thereby maintaining a state in which imaging surface S of imaging plate IP faces upward.

Further, conveying guides 36I and 36J, conveying roller pairs 28J and 28K, heat roller pair 248, and conveying roller pair 28L are disposed from the downstream end of conveying guide 36H in the conveying direction to the rear side of the device. Conveying guides 36I and 36J are disposed from the front side of the device to the rear side of the device in this order. Conveying roller pair 28J is disposed between conveying guide 36H and conveying guide 36I, and conveying roller pair 28K and heat roller pair 248 are disposed between conveying guide 36I and conveying guide 36J, and conveying roller pair 28L is disposed at the side of conveying guide 36J toward the rear of the device.

Namely, while imaging plate IP, that has been slidingly dropped onto conveying guide 36H, is guided by conveying guides 36I and 36J, imaging plate IP is conveyed toward the rear side of the device by conveying roller pairs 28J and 28K, heat roller pair 248 and conveying roller pair 28L.

Conveying guide 36K is disposed at the rear side of conveying roller pair 28K of the device. Conveying guide 38K is inclined toward the lower part of the device from the rear side of the device to the front side of the device, and imaging plate IP is guided (dropped) to the front lower side of the device with the leading end and the tail end of the imaging plate IP reversed, thereby maintaining a state in which imaging surface S of imaging plate IP faces upward.

Further, conveying guides 36L, 36M and 36N, conveying roller pairs 28M and 28N, pressure roller pair 250, and conveying roller pair 280 are respectively disposed in this order from the downstream end of conveying guide 36K in the conveying direction to discharge port 246 of the front side of the device. Conveying guides 36L, 36M and 36N are disposed from the rear side of the device to the front side of the device in this order. Conveying roller pair 28M is disposed between conveying guide 36K and conveying guide 36L, and conveying roller pair 28N and pressure roller pair 250 are disposed between conveying guide 36L and conveying guide 36M, and conveying roller pair 280 is disposed between conveying guide 36M and conveying guide 36N.

Namely, while imaging plate IP, that has been slidingly dropped onto conveying guide 36K, is guided by conveying guides 36L, 36M and 36N, imaging plate IP is conveyed by conveying roller pairs 28M and 28N, pressure roller pair 250 and conveying roller pair 280 toward the front side of the device, and discharged from discharge port 246.

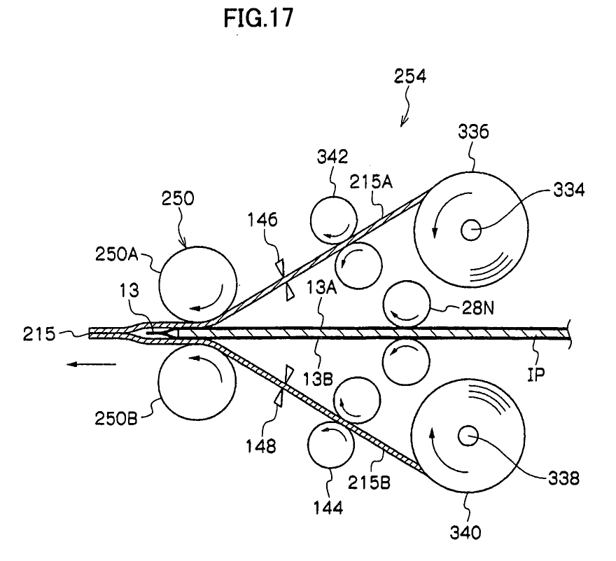

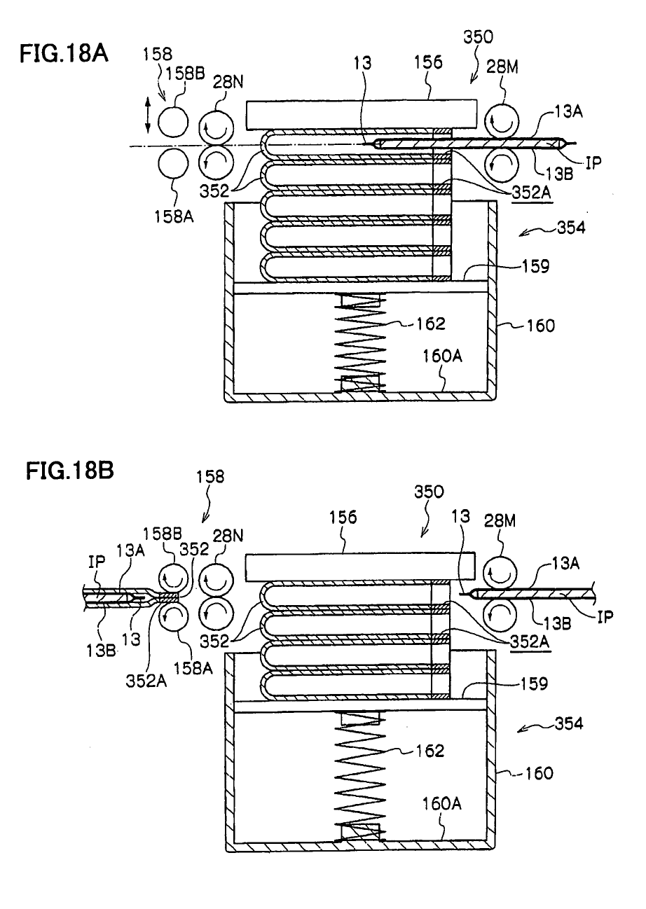

Further, within housing 222, protective case enclosure mechanism 252, which is a protective member attachment unit, and contamination-prevention pack enclosure mechanism 254, which is a pack enclosing unit, are arranged in this order from the upstream side in the conveying direction. Protective case enclosure mechanism 252 forms protective case 13 and encloses imaging plate IP within the formed protective case 13. In addition, contamination-prevention pack enclosure mechanism 254 forms contamination-prevention pack 215 (see FIGS. 17 and 18A and 18B) inside of which the protective case 13, having imaging plate IP enclosed therein, can be enclosed, and encloses protective case 13, inside of which imaging plate IP is enclosed, within the formed contamination-prevention pack 215.

Hereinafter, the operation of the embodiment will be described.

When imaging plate IP is inserted from insertion port 224 into housing 218, imaging plate IP is conveyed downward in the device by conveying roller pair 28A and passes through disinfection mechanism 234. At this time, imaging plate IP is stopped for a prescribed time to be disinfected and sterilized in the disinfection mechanism 234. Thereafter, disinfected imaging plate IP is conveyed downward in the device by conveying roller pair 28B, passes through discharge port 226 and is discharged from housing 218, and passes through insertion port 33 and is inserted into housing 220.

Imaging plate IP inserted into housing 220 is conveyed by conveying roller pair 28D, passes through a laser beam irradiation position (details of which are described below) of image reading mechanism 238, and an X-ray image carried on imaging surface S is read by image reading mechanism 238. The X-ray image read by image reading mechanism 238 is displayed on a monitor display screen.

Imaging plate IP, having passed through the laser irradiation position in image reading mechanism 238, is conveyed downward in the device by conveying roller pair 28E, passes through a light irradiation position (details of which are described below) in residual image erasing mechanism 240, and the X-ray image carried on imaging surface S is erased.

Imaging plate IP, having passed through the light irradiation position in residual image erasing mechanism 240, is conveyed downward in the device by conveying roller pair 28F, and imaging plate IP is guided to conveying roller pair 28G by conveying guides 36D and 36E. At this time, the leading end and the tail end of imaging plate IP are reversed by conveying guides 36D and 36E, so that imaging surface S of imaging plate IP faces upward.

Then, in a state in which imaging surface S of imaging plate IP faces upward, imaging plate IP is conveyed to the front side of the device by conveying roller pairs 28G and 28H, passes through discharge port 35, and is discharged from housing 220, and further, is inserted into housing 222 through insertion port 244.

After imaging plate IP inserted into housing 222 is conveyed to the front side of the device by conveying roller pair 28I, imaging plate IP is guided to conveying roller pair 28J by conveying guide 36H. At this time, the leading end and the tail end of imaging plate IP are transport by conveying guide 36H, so that imaging surface S of imaging plate IP faces upward.

Then, in a state in which imaging surface S of imaging plate IP faces upward, imaging plate IP is conveyed by conveying roller pairs 28J and 28K, passes through protective case enclosure mechanism 252, and is enclosed in protective case 13. After imaging plate IP, enclosed in protective case 13, is conveyed rearward in the device by heat roller pair 248 and conveying roller pair 28L, imaging plate IP, enclosed in protective case 13, is guided to conveying roller pair 28M by conveying guide 36K. At this time, the leading end and the tail end of imaging plate IP enclosed in protective case 13 are reversed, such that imaging surface S of imaging plate IP faces upward.

Then, imaging plate IP enclosed in protective case 13 is conveyed frontward in the device by conveying roller pairs 28M and 28N, and passes through contamination-prevention pack enclosure mechanism 254 to be enclosed in contamination-prevention pack 215. Then imaging plate IP and protective case 13 enclosed in contamination-prevention pack 215 are conveyed frontward in the device by pressure roller 250 and conveying roller pair 280, and pass through discharge port 246 to be discharged from housing 222.

Here, in the present embodiment, since imaging plate IP inserted into image reading device 11 is conveyed to image reading mechanism 238 after imaging plate IP has been disinfected by disinfection mechanism 234, it is possible to ensure that regions in the image reading device 11 at the downstream side of the disinfection mechanism 234 in the conveying direction through which the disinfected and sterilized imaging plate IP passes, are clean regions. Further, disinfection of imaging plate IP by an operator before imaging plate IP is inserted into image reading device 11 is not required. Accordingly, proliferation of bacteria within the image reading device 11 can be prevented, and an operator's workload can be reduced.

The suppression of proliferation of bacteria in image reading device 11 leads to suppression of adhesion and accumulation of bacteria at an optical system (details of which are described below) provided in the image reading mechanism 238, so that reduction of the reading capability of an X-ray image by image reading mechanism 238 can also be suppressed.

Further, disinfection mechanism 234 and the clean region at the downstream side of disinfection mechanism 234 in the conveying direction are partitioned into separate chambers by lower wall 218B of housing 218 and upper wall 220A of housing 220, so that invasion of bacteria into the clean region can be further suppressed and proliferation of bacteria in the clean region can also be further suppressed.

The chamber pressure within housing 220, accommodating image reading mechanism 238 and residual image erasing mechanism 240 therein, is controlled so as to be a predetermined value higher than atmospheric pressure by the chamber pressure control mechanism 242, whereas the chamber pressure within housing 220, accommodating disinfection mechanism 234 therein, is controlled so as to be equal to atmospheric pressure. Accordingly, invasion of bacteria from housing 218 to housing 220 is suppressed, and therefore, invasion of bacteria into the clean region is further suppressed.

Further, in this embodiment, housing 218, accommodating disinfection mechanism 234 therein, is detachably connected with housing 220 accommodating image processing section 212 (image reading mechanism 238 and residual image erasing mechanism 240) therein. Accordingly, if image processing section 212 is a conventional image reading device, it is possible to optionally add a disinfection function to the conventional image reading device.

Furthermore, in this embodiment, after an X-ray image on imaging plate IP has been erased by residual image erasing mechanism 240, imaging plate IP is enclosed in protective case 13 by protective case enclosing mechanism 252, and imaging surface S of imaging plate IP is covered with protective case 13.

As a result, it is unnecessary to manually enclose imaging plate IP discharged from image reading device 11 within protective case 13, and the workload of an operator can be reduced. Further, since imaging plate IP is discharged from image reading device 11 in a state in which imaging surface S is protected by protective case 13, contamination of imaging surface S of imaging plate IP can further be suppressed.

In this embodiment, after imaging plate IP has been enclosed in protective case 13 by protective case enclosing mechanism 252, imaging plate IP is enclosed in contamination-prevention pack 215 by contamination-prevention pack enclosure mechanism 254.

As a result, it is unnecessary to manually enclose imaging plate IP discharged from image reading device 11, in a state in which the imaging plate IP is enclosed in protective case 13, within contamination-prevention pack 215 and, therefore, the workload of the operator can be reduced. Furthermore, imaging plate IP is discharged from image reading device 11 in a state in which imaging plate IP enclosed in protective case 13 is further enclosed within contamination-prevention pack 215, so that not only contamination of imaging plate IP, but also contamination of protective case 13, can be prevented.

In this embodiment, insertion port 224 is separated from discharge port 246 so that disinfected imaging plate IP cannot be reinserted into housing 218. Accordingly, re-adhesion of bacteria to disinfected imaging plate IP can be prevented, and a clean imaging plate IP without re-adhesion of bacteria can be discharged from the device. However, it is not essential that insertion port 224 is separated from discharge port 246. Insertion port 224 may be the same as discharge port 246, and the conveying direction of imaging plate IP from which a residual image has been erased can be reversed to discharge imaging plate IP from insertion port 224. In this case, it is possible that the downstream side from disinfection mechanism 234 in the conveying direction can be provided as a clean region through which only a disinfected imaging plate IP can pass.

Further, in this embodiment, housing 220 and housing 218 are separate bodies, but even if housing 220 and housing 218 are configured as a single body, it is possible to prevent imaging plate IP after disinfection and imaging plate IP before disinfection from passing through the same path, by providing an insertion port and a discharging port separately, so that an effect similar to the above can be obtained.

(Disinfection Mechanism 234)

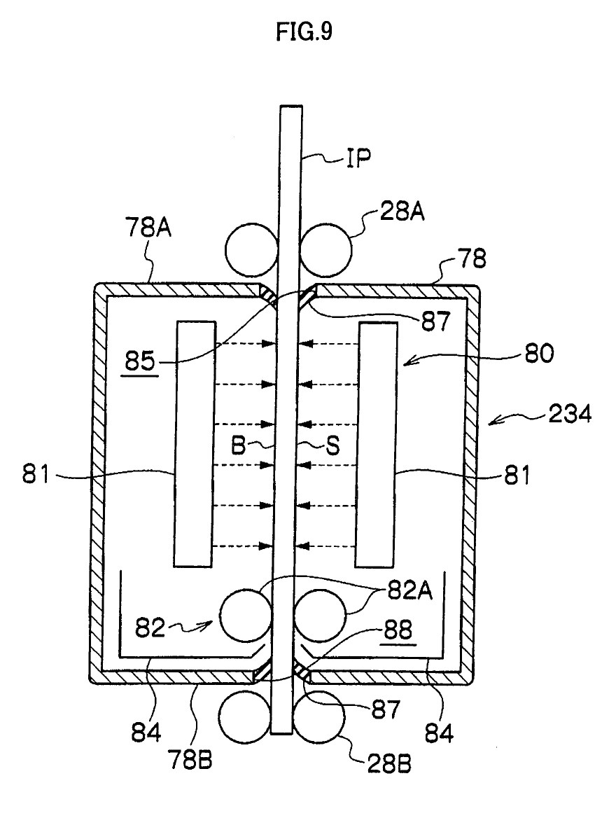

FIG. 9 shows a schematic sectional side view of disinfection mechanism 234. As shown in the drawing, disinfection mechanism 234 includes rectangular housing 78 as viewed from a lateral direction of the imaging plate (direction perpendicular to both the conveying direction and the thickness direction of the imaging plate), disinfection liquid ejection unit 80 provided along the imaging plate conveying direction in housing 78, squeeze roller pair 82, and a pair of disinfection liquid recovery sections 84 accommodating the respective rollers of the squeeze roller pair 82 therein.

Insertion port 85, into which imaging plate IP is inserted, is provided at upper wall 78A of housing 78, and discharge port 88 from which imaging plate IP is discharged is provided at lower wall 78B of housing 78, so that the imaging plate conveying path traverses vertically across the interior of housing 78.

Seal portions 87 made of elastic and waterproof material such as rubber are disposed at insertion port 85 and discharge port 88, respectively. At respective seal portions 87, an opening, through which an imaging plate IP being conveyed can pass, and which can maintain sealability between the imaging plate IP being conveyed and the seal portions 87 is provided.

Disinfection liquid ejection unit 80 is provided with a pair of ejection heads 81 which are disposed at the opposite side of the imaging plate conveying path from each other in the thickness direction of the imaging plate. Respective ejection heads 81 extend in the imaging plate conveying direction and the lateral direction of the imaging plate, and eject a disinfectant liquid such as alcohol to the entire surface of imaging surface S or rear surface B of imaging plate IP.

Further, rollers 82A of squeeze roller pair 82 are disposed at the opposite side of the imaging plate conveying path from each other in the thickness direction of the imaging plate. Rollers 82A are maintained in a stopped state or rotate in a reverse direction to the conveying direction to scrape off (squeeze) the disinfectant liquid adhered to imaging surface S or rear surface B of imaging plate IP.

Disinfection liquid recovery section 84 is a container for accommodating each roller 82A of squeeze roller pair 82, and recovers the disinfectant liquid dropped from each roller 82A. The waste disinfectant liquid may be stored in disinfection liquid recovery section 84, or stored in a separate recovery unit connected to disinfection liquid recovery section 84 via a drain pipe.

Next, operation of the disinfection mechanism 234 is explained.

Imaging plate IP conveyed by conveying roller pair 28A to the downward side of the device is stopped between the pair of ejection heads 81 for a prescribed period of time. During this period of time, the disinfectant liquid is ejected from ejection heads 81 to both of the front and rear surfaces of imaging plate IP, thereby disinfecting imaging surface S and rear surface B. When sterilized and disinfected imaging plate IP passes through squeeze roller pair 82, the disinfectant liquid adhered to imaging plate IP is scraped off by respective rollers 82A of squeeze roller pair 82. The disinfectant liquid scraped off from imaging plate IP drops from respective rollers 82A to disinfection liquid recovery section 84 and is recovered. As a result, imaging plate IP, which has been disinfected and from which disinfectant liquid has been scraped off, can be inserted into image processing section 212.

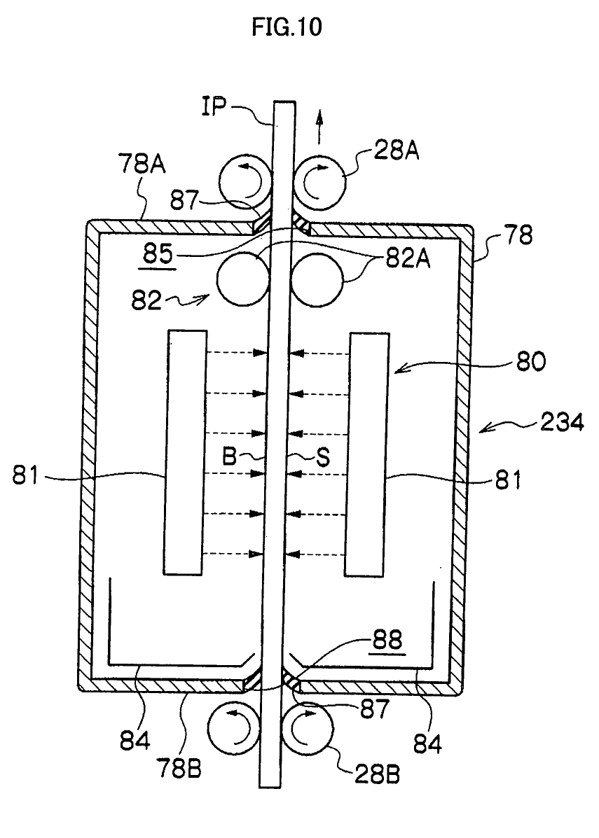

In this embodiment, the device is configured such that imaging plate IP is conveyed downward in the device to be passed through disinfection liquid ejection unit 80, but as shown in FIG. 10, the device may be configured such that imaging plate IP is conveyed upward in the device to be passed through disinfection liquid ejection unit 80. In this case, it is necessary to locate squeeze roller pair 82 above disinfection liquid ejection unit 80 in the device.

Here, when imaging plate IP is conveyed upward in the device to be passed through disinfection liquid ejection unit 80 and squeeze roller pair 82, it is possible that the disinfectant liquid scraped off from imaging plate IP by squeeze roller pair 82 drops by gravity. Accordingly, the capability of the scrape-off of the disinfectant liquid from imaging plate IP by squeeze roller pair 82 can be improved.

(First Modified Example of Disinfection Mechanism 234)

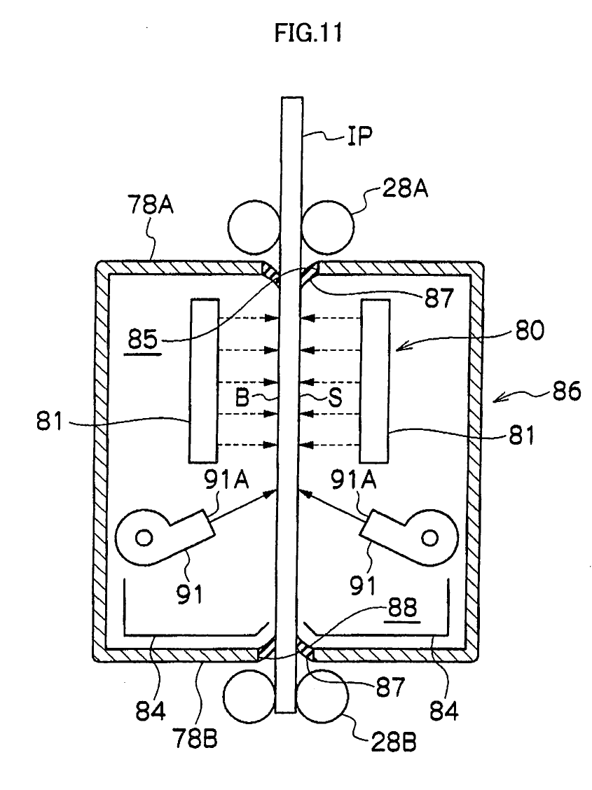

In FIG. 11, a schematic configuration of disinfection mechanism 86 as a first modified example of disinfection mechanism 234 is shown in sectional side view. As shown in this drawing, disinfection mechanism 86 has a pair of blowers (blowing unit) 91 in place of squeeze roller pair 82 in disinfection mechanism 234. The pair of blowers 91 is disposed between ejection heads 81 and disinfection liquid recovery section 84, and blowers 91 are disposed at the opposite side of the imaging plate conveying path from each other in the thickness direction of the imaging plate.

Blowing opening 91A of each blower 91 is directed toward the imaging plate conveying path side and obliquely upward, and the blowers 91 blow air toward imaging surface S or rear surface B of imaging plate IP being conveyed.

Next, the operation of the disinfection mechanism 86 is described.

The disinfectant liquid is ejected from a pair of ejection heads 81 to the entire surface of both of the front and rear surfaces of imaging plate IP being conveyed downward in the device by conveying roller pair 28A, thereby sterilizing and disinfecting imaging surface S and rear surface B of imaging plate IP. The pair of blowers 91 blow air from an obliquely downward side to imaging surface S and rear surface B of imaging plate IP, thereby blowing the disinfectant liquid adhered to imaging surface S and rear surface B of imaging plate IP upward in the device. As a result, it is possible to insert disinfected imaging plate IP into image processing section 212, with the disinfectant liquid having been removed therefrom.

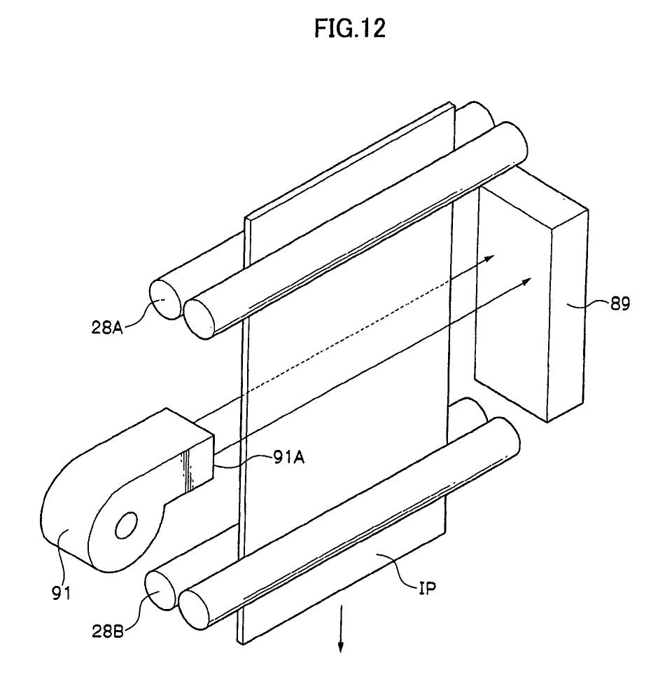

In this modified example, blowing openings 91A of blowers 91 are disposed opposite imaging surface S and rear surface B of imaging plate IP, respectively, but as shown in FIG. 12, blower 91 may disposed at an outer side of the imaging plate in the lateral direction of the imaging plate, and blowing opening 91A may be disposed opposite the side face of imaging plate IP. In this case, the disinfectant liquid adhered to imaging surface S and rear surface B of imaging plate IP is blown off to the outside in a the lateral direction of the imaging plate, so that re-adhesion to the imaging plate IP of the disinfectant liquid blown from imaging plate IP can be prevented. Furthermore, in this embodiment, as shown in the drawing, it is preferable that liquid absorbing member 89 having a liquid absorbing property such as a sponge is disposed at the opposite side of the imaging plate conveying path to blower 91 so that the disinfectant liquid blown from imaging plate IP is absorbed by liquid absorbing member 89, thereby reducing the workload for treatment of waste liquid.

(Second Modified Example of Disinfection Mechanism 234)

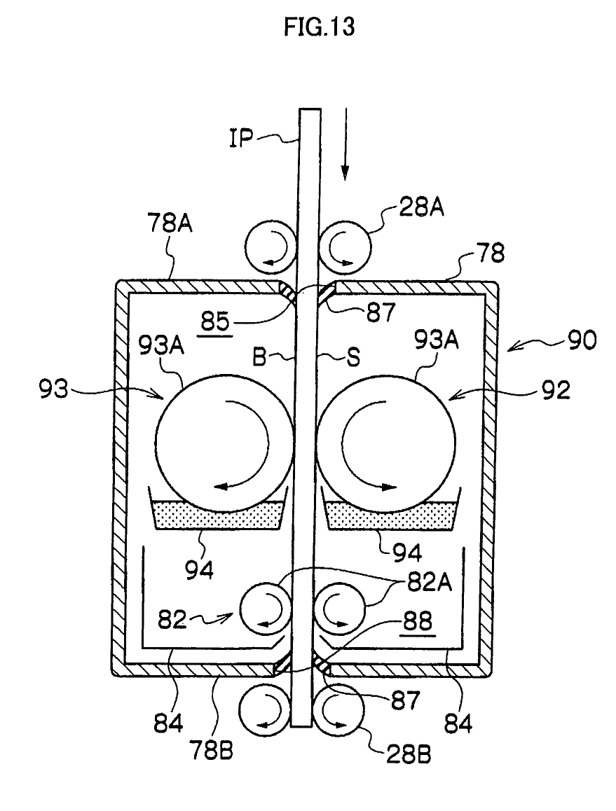

In FIG. 13, a schematic configuration of disinfection mechanism 90 as a second modified example of disinfection mechanism 234 is shown in sectional side view. As shown in this drawing, disinfection mechanism 90 has disinfection liquid applying section 92 in place of disinfection liquid ejection unit 80 in disinfection mechanism 234. Disinfection liquid applying section 92 includes disinfection liquid applying roller pair 93 and a pair of disinfection liquid storing sections 94.

Rollers 93A constituting disinfectant liquid coating roller pair 93 are disposed at the opposite side of the imaging plate conveying path from each other in the thickness direction of the imaging plate, and are formed by a liquid absorbing material such as sponge. In disinfection liquid storing section 94, a disinfection liquid such as alcohol is stored.

The lower portion of each roller 93A is immersed in the disinfectant liquid stored in disinfection liquid storing section 94, thereby each roller 93A is impregnated with disinfection liquid. Here, in this embodiment, disinfection liquid applying roller pair 93 are driven rollers, but may be drive rollers.

Next, operation of disinfection mechanism 90 is described.

Disinfectant liquid coating roller pair 93 is driven and dependently rotated by imaging plate IP conveyed by conveying roller pair 28A to a lower part of the device. Here, each roller 93A constituting disinfectant liquid coating roller pair 93 is impregnated with disinfectant liquid so that the disinfectant liquid is coated onto imaging surface S and rear surface B of imaging plate IP to sterilize and disinfect imaging plate IP.

Thereafter, the disinfectant liquid adhered to sterilized and disinfected imaging plate IP is scraped off by squeeze roller pair 82 and recovered in disinfection liquid recovery section 84. As a result, it is possible to insert imaging plate IP into image processing section 212 with imaging plate IP having been disinfected and having had the disinfectant liquid removed therefrom.

Here, in this embodiment, since each roller 93A constituting disinfection coating roller pair 93 is formed by a liquid absorbent member, dirt such as saliva and blood adhered to imaging surface S and rear surface B of imaging plate IP can be absorbed and removed by each roller 93A. In other words, disinfection coating roller pair 93 additionally has a cleaning function for cleaning imaging plate IP.

(Third Modified Example of Disinfection Mechanism 234)

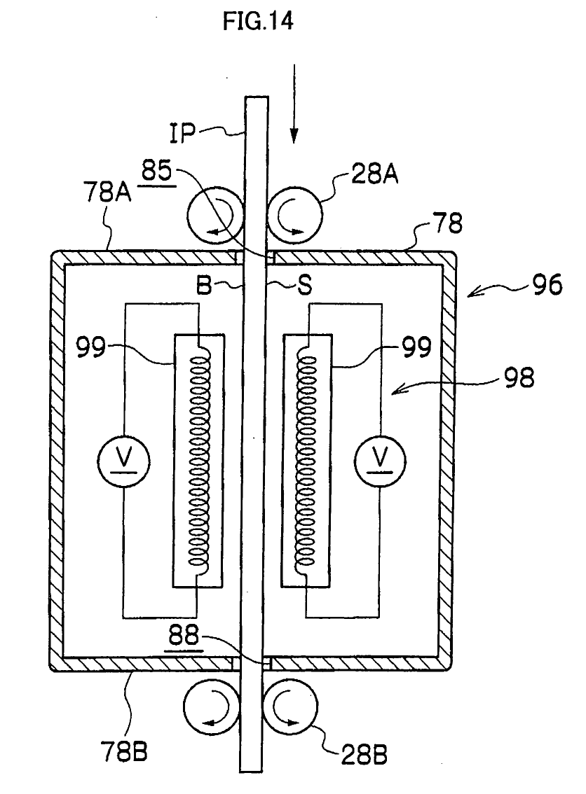

In FIG. 14, a schematic configuration of disinfection mechanism 96 as a third modified example of disinfection mechanism 234 is shown in sectional side view. As shown in this drawing, disinfection mechanism 96 has heating disinfection unit 98. Hating disinfection unit 98 includes a pair of heaters 99 disposed at the opposite side of the imaging plate conveying path from each other in the thickness direction of the imaging plate. Each heater is disposed so as to face the entire area of imaging plate IP in the lateral direction, and the entire area of the imaging plate IP being conveyed is heated by heaters 99.

Next, the operation of disinfection mechanism 96 is described.

Imaging plate IP is conveyed toward the bottom of the device by conveying roller pair 28A to pass through heating disinfection unit 98. At this time, imaging surface S and rear surface B of imaging plate IP are heated to be sterilized and disinfected. Therefore, a sterilized and disinfected imaging plate IP can be inserted into image processing section 212.

In this embodiment, since disinfectant liquid is not used, a mechanism such as squeeze roller pair for removing disinfectant liquid from imaging plate IP is not required, and further, it is not necessary for insertion port 85 and discharge port 88 of housing 78 to be waterproof, thereby achieving simplification of the disinfection mechanism.

(Image Reading Mechanism 238)

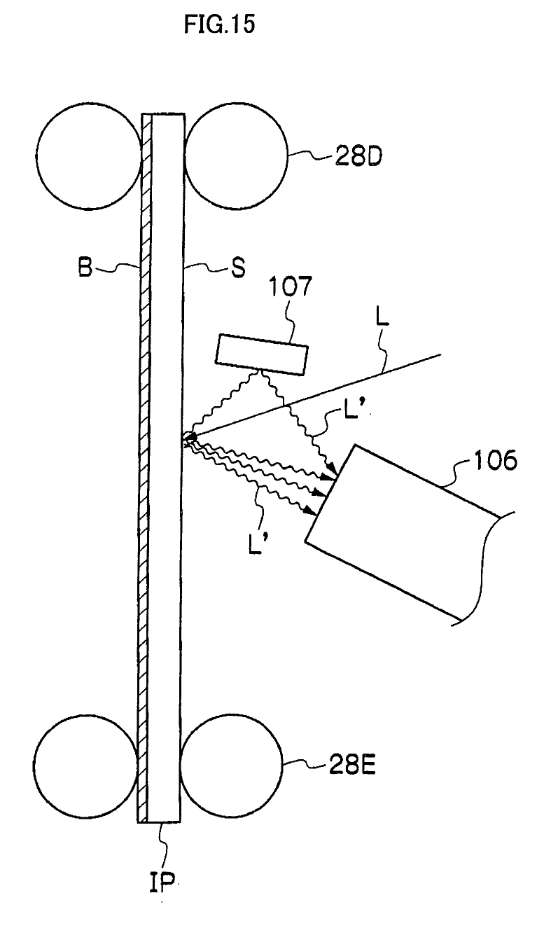

As shown in FIG. 6, image reading mechanism 238 includes optical scanning device 102, light-converging guide 106, light-converging mirror 107 (see FIG. 15) and photoelectric conversion section (photomultiplier) 108. Optical scanning device 102 includes at least device housing 310 disposed further toward a rear side of the device than imaging plate IP and having a longitudinal direction that is the vertical direction of the device, light source portion 312, deflector (polygon mirror) 314 and reflection mirror 316 housed in device housing 310.

Reflection mirror 316, light source portion 312 and deflector 314 are arranged in this order from the upper portion to the lower portion of the device. Light source portion 312 emits laser beam L toward a rearward and downward direction of the device, deflector 314 reflects laser beam L toward a rearward and upward direction of the device, and deflects the beam in the lateral direction of the imaging plate. After laser beam L deflected by deflector 314 is condensed and diffused by an optical element (not shown), the laser beam is reflected to the area between conveying roller pair 28D and conveying roller pair 28E by reflection mirror 316 to scan imaging surface S of imaging plate IP being conveyed between the conveying roller pair 28D and the conveying roller pair 28E.

As shown in FIG. 15, when imaging surface S (photo-stimulable phosphor layer) of imaging plate IP is irradiated with a laser beam L as excitation light, photo-stimulated luminescence light L′ takes place in response to the energy stored in imaging surface S, namely, corresponding to an image.

Light-converging guide 106 and light-converging mirror 107 are disposed in the vicinity of imaging plate IP in the main scanning direction of imaging surface S of imaging plate IP, and photo-stimulated luminescence light L′ caused on imaging surface S is guided to photoelectric conversion section 108. Photoelectric conversion section 108 converts photo-stimulated luminescence light L′ obtained from imaging plate IP to an electrical signal.

(Residual Image Erasing Mechanism 240)

As shown in FIGS. 6 and 7, residual image erasing mechanism 240 comprises erasing lamp 318, such as a cathode tube or a fluorescent lamp, disposed so as to face imaging surface S of imaging plate IP being conveyed. Erasing lamp 318 irradiates to imaging surface S an erasing light including light in the excitation wavelength region of the phosphor constituting imaging surface S of imaging plate IP. In this way, X-ray energy remaining in imaging surface S of imaging plate IP is erased and an X-ray image remaining in imaging surface S is erased.

(Protective Case Enclosure Mechanism 252)

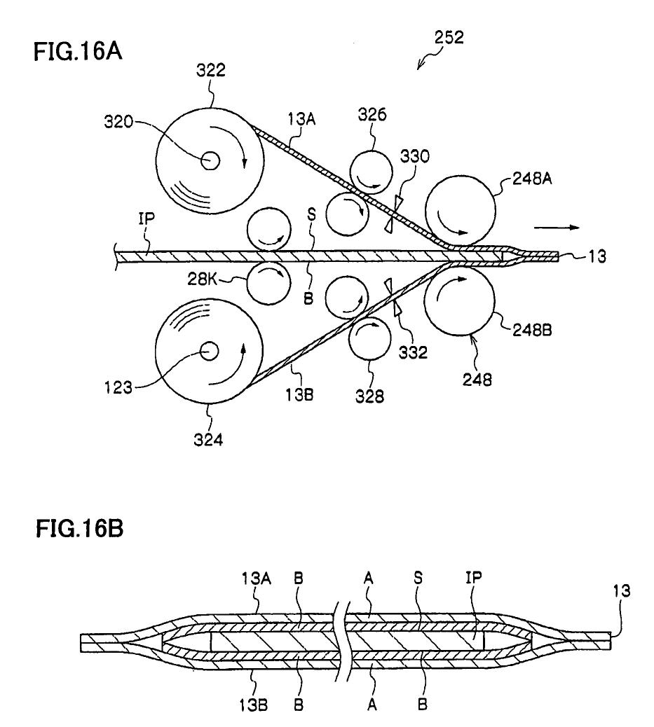

In FIG. 16A, a schematic configuration of protective case enclosure mechanism 252 is shown in sectional side view. As shown in this drawing, protective case enclosure mechanism 252 comprises roll body 322 formed by winding sheet member 13A around winding core 320, roll body 324 formed by winding sheet member 13B around winding core 123, heat roller pair 248 disposed from the downstream side of roll bodies 322 and 324 in the conveying direction, unwind roller pair 326 for unwinding sheet member 13A from the roll body 322, unwind roller pair 328 for unwinding sheet member 13B from roll body 324, cutter 330 for cutting sheet member 13A, and cutter 332 for cutting sheet member 13B.

Roll body 322 is disposed opposite imaging surface S of imaging plate IP along the lateral direction of the imaging plate, and roll body 324 is substantially parallel to roll body 322 and disposed at the opposite side of the imaging plate conveying path from roll body 322 in the thickness direction of the imaging plate.

Further, heat roller pair 248 is constituted by heat roller 248A disposed at the imaging surface S side, and pressure roller 248B which is disposed at the rear surface B side and press-contacted against heat roller 248A.

Unwind roller pair 326 is disposed between roll body 322 and heat roller 248A, and nips sheet member 13A and conveys sheet member 13A between heat roller 248A and imaging plate IP. Further, unwind roller pair 328 is disposed between roll body 324 and pressure roller 248B, and nips sheet member 13B and conveys sheet member 13B between pressure roller 248B and imaging plate IP.

Cutter 330 is disposed between unwind roller pair 326 and heat roller 248A, and is driven at a predetermined timing to cut sheet member 13A to a prescribed length. Further, cutter 332 is disposed between unwind roller pair 328 and pressure roller 248B, and is driven at a predetermined timing to cut sheet member 13B to a prescribed length.

Here, as shown in FIG. 16B, sheet members 13A and 13B have a laminated structure comprising thermoplastic layer A continuously formed by a thermoplastic material in the longitudinal direction (take-up and unwind directions) and thermosetting layer B formed by a thermosetting material on thermoplastic layer A. Thermosetting layer B is formed on thermoplastic layer A at prescribed intervals in the longitudinal direction. Furthermore, thermosetting layer B is formed in a rectangular shape and is slightly larger than the size of imaging plate IP. The width of thermoplastic layer A in the lateral direction is wider than the width of thermosetting layer B in the lateral direction so that both edge portions of thermoplastic layer A are exposed to air. In addition, thermoplastic layer A is exposed to air between subsequent thermosetting layers B, and thermoplastic layer A is cut by cutters 330 and 332 at this portion.

Next, operation of protective case enclosure mechanism 252 is described.

When imaging plate IP is conveyed to heat roller pair 248 by conveying roller pair 28K, sheet member 13A is unwound from roll body 322 by unwind roller pair 326, and sheet member 13B is unwound from roll body 324 by unwind roller pair 328. At this time, unwind roller pairs 326 and 328 align the phase of thermosetting layer B of sheet member 13B with that of sheet member 13A, and convey sheet member 13A and sheet member 13B.

Unwind roller pairs 326 and 328 convey sheet members 13A and 13B such that the leading end of thermosetting layer B reaches the nip portion of heat roller pair 248 before the leading end of imaging plate IP reaches the nip portion of heat roller pair 248.

In this way, first, the leading ends of sheet member 13A and sheet member 13B are pressed and heated by heat roller pair 248. Since the leading ends of sheet member 13A and sheet member 13B are only formed from thermoplastic layer A, the leading ends are bonded to each other by being pressed and heated.

Thereafter, sheet member 13A and sheet member 13B are sequentially pressed and heated from the leading end to the tail end thereof by heat roller pair 248. Since the opposite sides of sheet member 13A and sheet member 13B in the widthwise direction, and at the tail end thereof are only formed from thermoplastic layer A, these portions are bonded to each other by being pressed and heated by heat roller pair 248.

Here, since at portions where sheet member 13A and sheet member 13B overlap imaging plate IP, thermoplastic layer A overlaps imaging plate IP via thermosetting layer B, sheet members 13A, 13B and imaging plate IP are not adhered, even if the portions are pressed and heated by heat roller pair 248.

In this way, the entire peripheral portion of sheet members 13A, 13B can be bonded to each other without adhering the sheet member 13A and sheet member 13B to the imaging plate IP. Accordingly, protective case 13 capable of enclosing imaging plate IP can be produced, and imaging plate IP can be enclosed within protective case 13.

Further, the configuration of this mechanism is also applicable to a mechanism for enclosing imaging plate IP, which is enclosed in protective case 13, within a contamination-prevention pack.

(Contamination-Prevention Pack Enclosure Mechanism 254)

In FIG. 17, a schematic configuration of contamination-prevention pack enclosure mechanism 254 is shown in sectional side view. As shown in this drawing, contamination-prevention pack enclosure mechanism 254 comprises roll body 336 formed by winding sheet member 215A around winding core 334, roll body 340 formed by winding sheet member 215B around winding core 338, pressure roller pair 250 disposed at the downstream side in the conveying direction from roll bodies 336 and 340, unwind roller pair 342 for unwinding sheet member 215A from roll body 336, unwind roller pair 144 for unwinding sheet member 215B from roll body 340, cutter 146 for cutting sheet member 215A, and cutter 148 for cutting sheet member 215B.

Roll body 336 is opposite sheet member 215A of contamination-prevention pack 215 and disposed along the lateral direction of the imaging plate, and roll body 340 is substantially parallel to roll body 336 and disposed at the opposite side of the imaging plate conveying path from roll body 336.

Pressure roll pair 250 is structured by drive roller 250A disposed at the sheet member 215A side, and pressure roller 250B which is disposed at the sheet member 215B side and press-contacted against drive roller 250A.

Unwind roller pair 342 is disposed between roll body 336 and drive roller 250A, and nips sheet member 215A and conveys sheet member 215A between drive roller 250A and sheet member 13A. Further, unwind roller pair 144 is disposed between roll body 340 and pressure roller 250B, and nips sheet member 215B and conveys sheet member 215B between pressure roller 250B and sheet member 13B.

Further, cutter 146 is disposed between unwind roller pair 342 and drive roller 250A, and is driven at a predetermined timing to cut sheet member 215A to a prescribed length. Furthermore, cutter 148 is disposed between unwind roller pair 144 and pressure roller 250B, and is driven at a predetermined timing to cut sheet member 215B to a prescribed length.

Here, sheet members 215A and 215B are made of nylon resins or the like, and an adhesive layer is formed on the respective opposing surfaces thereof. The adhesive layer is formed in a grid-like shape by frame portions extending along the both side edges in the widthwise direction of sheet members 215A and 215B, and a plurality of ladder portions connected to the frame portions.

The size of rectangular areas encompassed with the adhesive layers on sheet members 215A and 215B is slightly larger but substantially the same size as the size of protective case 13. The ladder portions of sheet members 215A and 215B are cut by cutters 146 and 148 to be separated at a prescribed length.

Next, operation of contamination-prevention pack enclosure mechanism 254 is described.

When imaging plate IP enclosed in protective case 13 is conveyed to pressure roller pair 250 by conveying roller pair 28N, sheet member 215A is unwound from roll body 336 by unwind roller pair 342, and sheet member 215B is unwound from roll body 340 by unwind roller pair 144. At this time, unwind roller pairs 342 and 144 align the phase of the adhesive layers of sheet member 215A and sheet member 215B, and convey sheet member 13A and sheet member 13B.

Unwind roller pairs 342 and 144 convey sheet members 215A and 215B such that the ladder portion of the adhesive layer reaches the nip portion of pressure roller pair 250 before the leading end of protective case 13 reaches the nip portion of pressure roller pair 250.

In this way, first, the leading ends of sheet member 215A and sheet member 215B are pressed to each other by pressure roller pair 250. Since the adhesive layers are formed at the opposing surfaces of the leading ends of sheet member 215A and sheet member 215B, the leading ends of sheet member 215A and sheet member 215B are bonded to each other by being pressed by pressure roller pairs 250.

Thereafter, sheet member 215A and sheet member 215B are sequentially pressed from the leading end to the tail end thereof by pressure roller pair 250. Since the adhesive layers are formed at both side portions of sheet member 215A and sheet member 215B in the widthwise direction, and at the tail end thereof, both side portions of sheet member 215A and sheet member 215B in the widthwise direction, and the tail end thereof are bonded to each other by being pressed by pressure roller pair 250.

Here, since the adhesive layers are not formed at the portions where sheet member 215A and sheet member 215B overlap protective case 13, sheet members 215A, 215B and protective case 13 are not bonded to one another, even if these portions are pressed by pressure roller pair 250.

In this way, the entire peripheral portion of sheet members 215A and 215B can be bonded to each other without bonding sheet members 215A, 215B and protective case 13 to one another. Accordingly, contamination-prevention pack 215 capable of enclosing imaging plate IP which is enclosed in protective case 13 can be produced, and imaging plate IP enclosed in protective case 13 can be enclosed in contamination-prevention pack 215.

Further, the configuration of this mechanism is also applicable to a mechanism for enclosing imaging plate IP in protective case 13.

(Modified Example of Contamination-Prevention Pack Enclosure Mechanism 254)

In FIGS. 18A and 18B, a schematic configuration of a modified example of contamination-prevention pack enclosure mechanism 350 of contamination-prevention pack enclosure mechanism 254 is shown in sectional side view. As shown in this drawing, contamination-prevention pack enclosure mechanism 350 comprises contamination-prevention pack holding unit 354 disposed under the imaging plate conveying path extending substantially in the horizontal direction, stopper 156 disposed at the opposite side of the imaging plate conveying path from contamination-prevention pack holding unit 354, and heat roller pair 158 disposed at the downstream side of contamination-prevention pack holding unit 354 and stopper 156 in the imaging plate conveying direction.

Contamination-prevention pack holding unit 354 comprises rectangular plate-shaped stage 159 on which a plurality of contamination-prevention packs 352 are loaded, rectangular cylinder portion 160 having a bottom for slidably supporting stage 159 in the imaging plate thickness direction, and elastic member (compression coil spring) 162 which is provided between bottom portion 160A of cylinder portion 160 and stage 159 to urge stage 159 toward the imaging plate conveying path side.

Stopper 156 is a rectangular plate-shaped member, and is disposed at the opposite side of the imaging plate conveying path from stage 159. Contamination-prevention packs 352 placed on stage 159 are press-contacted against stopper 156 by the urging force of elastic member 162.

The height of stopper 156 is set such that the uppermost contamination-prevention pack 352 of plural contamination-prevention packs 352 is positioned at the imaging plate conveying path. Contamination-prevention pack 352 is a rectangular bag body capable of accommodating an imaging plate therein. The contamination-prevention packs 352 are placed on stage 159 such that one side of the bag body is opening 352A which becomes the tail end of the bag body. Further, the hardness of opening 352A is set to such an extent that opening 352A is maintained in a state in which opening 352A is opened as long as a locally large load such as pressure by a roller pair is not applied to opening 352A.

Further, heat roller pair 158 is composed of drive roller 158A disposed under the imaging plate conveying path and heat roller 158B disposed at the opposite side of the imaging plate conveying path from drive roller 158A. Heat roller 158B is capable of approaching and moving away from drive roller 158A.

Furthermore, a thermoplastic layer made of thermoplastic resin is formed on the inner peripheral surface of opening 352A of contamination-prevention pack 352.

Next, operation of contamination-prevention pack enclosure mechanism 350 is described.

When imaging plate IP enclosed in protective case 13 is conveyed to contamination-prevention pack enclosure mechanism 350 by conveying roller pair 28M, protective case 13 and imaging plate IP are inserted from opening 352A into contamination-prevention pack 352. Protective case 13 and imaging plate IP inserted into contamination-prevention pack 352 move toward the base portion side of the contamination-prevention pack 352 by inertial force even after departing from conveying roller pair 28M, and abut against the base portion to move the contamination-prevention pack 352 toward the downstream side in the conveying direction.

After the uppermost contamination-prevention pack 352 has been moved from stage 159 toward the downstream side in the conveying direction, stage 159 is pushed up by the urging force of elastic member 162, and the subsequent uppermost contamination-prevention pack 352 is placed on the imaging plate conveying path.

Thereafter, the bottom portion of contamination-prevention pack 352 is inserted into the nip portion of conveying roller pair 28N, and contamination-prevention pack 352, and protective case 13 and imaging plate IP enclosed therein, are conveyed toward the downstream side in the conveying direction by conveying roller pair 28N.

Heat roller 158B, in a state in which the nip between heat roller 158B and drive roller 158A is released, stands ready to receive opening 352A of contamination-prevention pack 352, and approaches drive roller 158A at the same time as opening 352A arrives at the nip position of heat roller 158B with drive roller 158A to form the nip portion between heat roller 158B and drive roller 158A.

In this way, opening 352A, having a thermoplastic layer formed on the inner peripheral surface of opening 352A, is pressed and heated by drive roller 158A and heat roller 158B so that the opposing surfaces at opening 352A in the vertical direction are bonded to each other to close opening 352A. Accordingly, imaging plate IP enclosed in protective case 13 is enclosed in contamination-prevention pack 352.

### THIRD EMBODIMENT

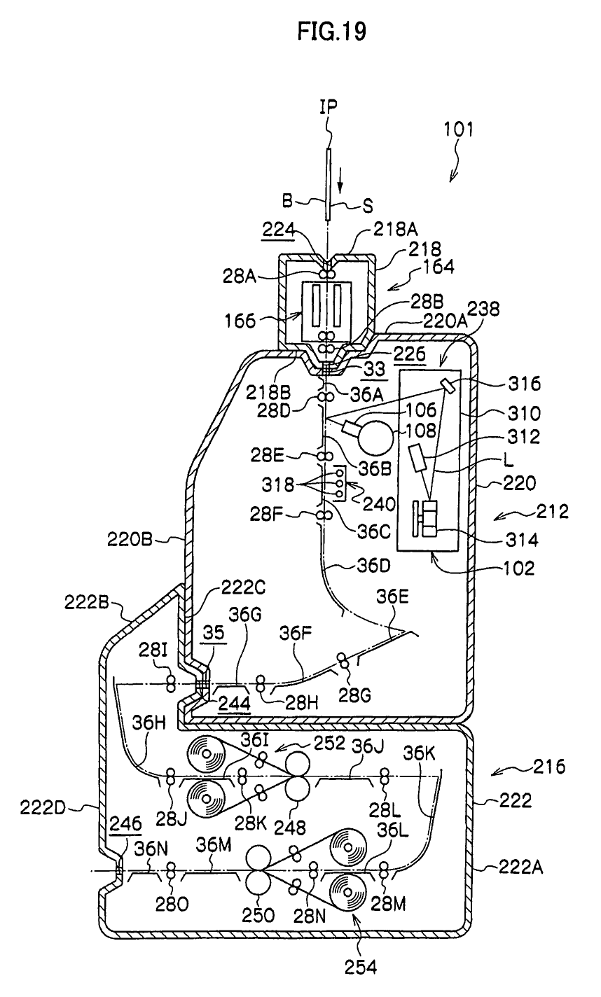

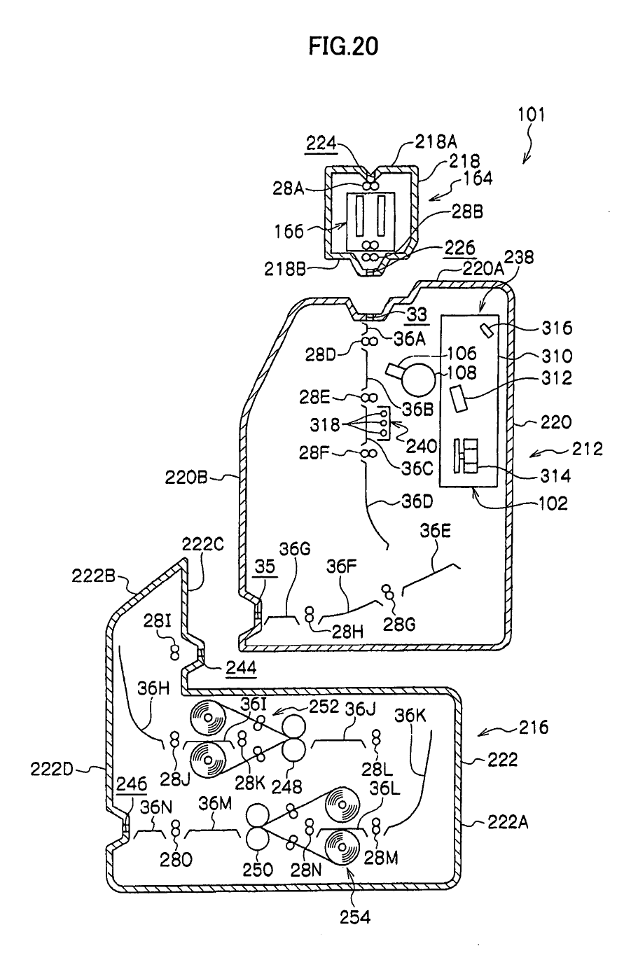

FIGS. 19 and 20 show sectional side views of schematic configurations of image reading device 101 according to a third embodiment. As shown in these drawings, image reading device 101 comprises image processing section 212, image pre-processing unit 164 and image post-processing section 216. Image pre-processing unit 164 is housed in housing 218, and housing 218 and housing 220 are detachably connected with each other to be integrated with image processing section 212.

Image pre-processing unit 164 is provided with cleaning mechanism 166, which is a cleaning unit, between conveying roller pair 28A and conveying roller pair 28B. Cleaning mechanism 166 cleans imaging plate IP to remove contaminants such as saliva and blood adhered to imaging plate IP.

Hereinafter, the operation of the embodiment will be described.

When imaging plate IP is inserted from insertion port 224 into housing 218, imaging plate IP is conveyed downward in the device by conveying roller pair 28A and passes through cleaning mechanism 166. At this time, imaging plate IP is cleaned by cleaning mechanism 166 to remove contaminants such as saliva and blood adhered to the outer periphery of the imaging plate. Then, cleaned imaging plate IP is conveyed downward in the device by roller pair 28B, passes through discharge port 226 and is discharged from housing 218, and passes through insertion port 33 to be inserted into housing 220.

As in the second embodiment, when imaging plate IP inserted into housing 220 passes through a laser irradiation position of image reading mechanism 238, an X-ray image carried on imaging surface S is read by image reading mechanism 238. When imaging plate IP passes through a light irradiation position in residual image erasing mechanism 240, the X-ray image carried on imaging surface S is erased. Thereafter, imaging plate IP is discharged from housing 220, and inserted into housing 222.

As in the second embodiment, when imaging plate IP inserted into housing 222 passes through protective case enclosure mechanism 252, imaging plate IP is enclosed in protective case 13. When imaging plate IP passes through contamination-prevention pack enclosure mechanism 254, imaging plate IP is enclosed in a contamination-prevention pack 215, and passes through discharge port 246 to be discharged from housing 222.

Here, in this embodiment, imaging plate IP inserted into image reading device 101 is cleaned by cleaning mechanism 166, and is conveyed to image reading mechanism 238 after contaminants such as saliva and blood adhered to the outer periphery of the imaging plate have been removed from imaging plate IP.

As a result, it is possible for an X-ray image carried on a cleaned imaging plate IP to be read by image reading mechanism 238. In addition, cleaning of imaging plate IP by an operator, prior to insertion into image reading device 101, becomes unnecessary. Accordingly, reduction in the reading performance of an X-ray image by image reading mechanism 238 can be suppressed and the operator's workload can be reduced.

Housing 218 housing cleaning mechanism 166 therein can be freely detachably connected with housing 220 in which image reading mechanism 238 and residual image erasing mechanism 240 are accommodated. Therefore, when image reading unit 212 is a conventional image reading device which is not provided with cleaning mechanism 166, a cleaning function can optionally be added to the conventional image reading device.

Further, in this embodiment, insertion port 224 is separated from discharge port 35 so that a cleaned imaging plate IP cannot be reinserted into housing 218. Accordingly, re-adhesion of contaminants to a cleaned imaging plate IP can be prevented, and a clean imaging plate IP without contaminants re-adhered thereto can be discharged from the device. However, it is not essential that insertion port 224 is separated from discharge port 35. Insertion port 224 may be the same as discharge port 35, and the conveying direction of imaging plate IP from which a residual image has been erased may be reversed to discharge imaging plate IP from insertion port 224.

(Cleaning Mechanism 166)

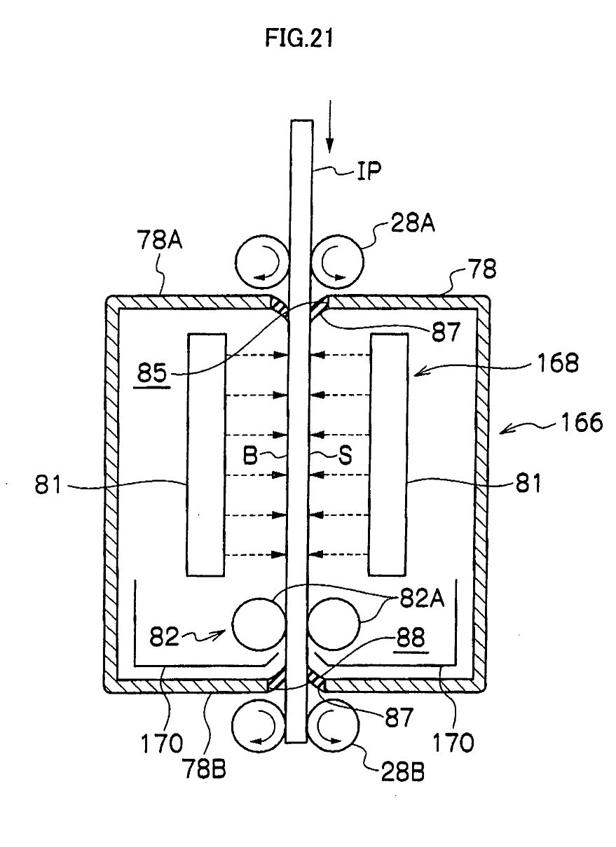

FIG. 21 shows a sectional side view of the schematic structure of cleaning mechanism 166. As shown in this figure, cleaning mechanism 166 has housing 78, cleaning liquid ejection section 168 disposed along the conveyance direction in housing 78, squeeze roller pair 82, and a pair of cleaning liquid recovery sections 170 that house respective rollers 82A of squeeze roller pair 82. The structure is similar to disinfection mechanism 234 although the liquid to be used is a cleaning liquid rather than a disinfectant liquid.

The operation of cleaning mechanism 166 is described in the following.

When imaging plate IP conveyed toward the bottom of the device by conveying roller pair 28A passes between a pair of ejection heads 81, the pair of ejection heads 81 eject cleaning liquid (e.g., water) to both surfaces (front and rear surfaces) of imaging plate IP, so that imaging surface S and rear surface B of imaging plate IP are cleaned and so that contaminants, such as saliva or blood, adhered to imaging surface S and rear surface B of imaging plate IP are removed. Further, when cleaned imaging plate IP passes squeeze roller pair 82, cleaning liquid remaining on imaging plate IP is scraped off by respective rollers 82A of squeeze roller pair 82. The cleaning liquid that is scraped off imaging plate IP by respective rollers 82A flows from respective rollers 82A down to cleaning liquid recovery sections 170, and is recovered. As the result, it is possible to insert, into image processing section 212, a cleaned imaging plate IP from which the cleaning liquid is removed.

(First Modified Example of Cleaning Mechanism 166)

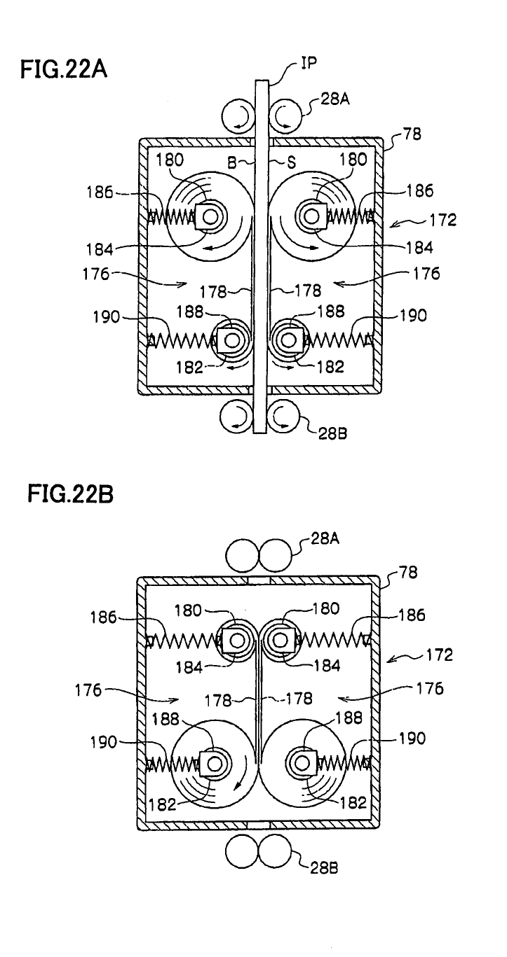

FIGS. 22A and 22B are sectional side views showing a schematic structure of cleaning mechanism 172, which is a first modified example of cleaning mechanism 166. As shown in this figure, cleaning mechanism 172 has housing 78 and a pair of cleaning web units 176 which are disposed to face each other in the thickness direction of the imaging plate with imaging plate conveyance path disposed therebetween.

Each of cleaning web units 176 has: web 178 formed of a water-absorbing member such as a sponge; winding core 180 which extends along the transverse direction of the imaging plate and which is wound with one end side of web 178 in a roll-shape; winding core 182 which is disposed substantially parallel to winding core 180 at the downstream side of winding core 180 with respect to the conveyance direction and which is wound with the other end side of web 178 in a roll-shape; bearing 184 which rotatably supports winding core 180; urging member (compression coil spring) 186 which urges bearing 184 toward the imaging plate conveyance path side; bearing 188 which rotatably supports winding core 182; and urging member (compression coil spring) 190 which urges bearing 188 toward the imaging plate conveyance path side.

The pair of winding cores 180 face each other in the thickness direction of the imaging plate with the imaging plate conveyance path disposed therebetween. The one-end sides of the pair of webs 178 are press-contacted with each other due to the urging force of urging members 186. The pair of winding cores 182 face each other in the direction of the thickness direction of the imaging plate with the imaging plate conveyance path disposed therebetween. The other-end sides of the pair of webs 178 are press-contacted with each other due to the urging force of urging members 190.

The operation of cleaning mechanism 172 is described below.

When imaging plate IP conveyed toward the bottom of the device by the pair of transport rollers 28A passes between the pair of webs 178, webs 178 are unwound from winding cores 180 and are wound around winding cores 182 due to rotation of winding rollers 180 and 182 driven by the movement of imaging plate IP. During this process, the pair of webs 178 contact both of the front and rear surfaces of imaging plate IP and absorb the water remaining on imaging surface S and rear surface B of imaging plate IP, whereby imaging surface S and rear surface B of imaging plate IP are cleaned and contaminants, such as saliva or blood, adhered to imaging surface S and rear surface B of imaging plate IP are removed. As a result, it is possible to insert a cleaned imaging plate IP into image processing section 212.

(Second Modified Example of Cleaning Mechanism 166)

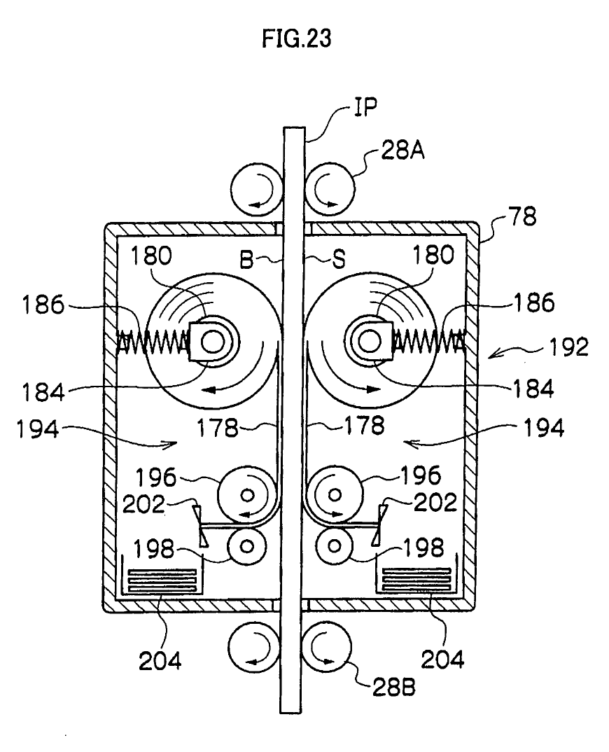

FIG. 23 is a sectional side view of a schematic structure of cleaning mechanism 192, which is a second modified example of cleaning mechanism 166. As shown in this figure, cleaning mechanism 192 has housing 78 and a pair of cleaning web units 194 which are disposed to face each other in the thickness direction of the imaging plate with the imaging plate conveyance path disposed therebetween.

Each of cleaning web units 194 has: web 178; winding core 180; drive roller 196 which is disposed substantially parallel to winding core 180 at the downstream side of winding core 180 with respect to the conveyance direction; bearing 184 which rotatably supports winding core 180; urging member (compression coil spring) 186 which urges bearing 184 toward the imaging plate conveyance path side; driven roller 198 which is disposed substantially parallel to drive roller 196 at the downstream side of drive roller 196 with respect to the conveyance direction and which is rotated according to the rotation of drive roller 196; cutter 202 which cuts the other end side (front end side) of web 178 conveyed by drive roller 196 and driven roller 198; and web recovery section 204 which recovers web 178 cut by cutter 202.

The pair of drive rollers 196 rotate while nipping imaging plate IP and the pair of webs 178, thereby unwinding webs 178 from winding cores 180. Webs 178 are conveyed away from the imaging plate conveyance path by drive rollers 196, and driven rollers 198 disposed below drive rollers 196.

Cutters 202 are disposed farther from the imaging plate conveyance path than drive rollers 196 and driven rollers 198, and cut the other end sides of webs 178 conveyed by drive rollers 196 and driven rollers 198 at a predetermined length. Web recovery sections 204 are disposed below the opposite sides of cutters 202 to the imaging plate conveyance path. Webs 178 drop into and are collected by web recovery sections 204 after webs 178 are cut to the predetermined length by cutters 202.

The operation of cleaning mechanism 192 is described next.

When imaging plate IP conveyed to the bottom of the device by the conveying roller pairs 28A passes between the pair of webs 178, webs 178 are unwound from winding cores 180 by drive rollers 196. During the process, the pair of webs 178 contact both of the front and rear surfaces of imaging plate IP and absorb the water remaining on imaging surface S and rear surface B of imaging plate IP, whereby imaging surface S and rear surface B of imaging plate IP are cleaned and contaminants, such as saliva or blood, adhered to imaging surface S and rear surface B of imaging plate IP are removed. As a result, it is possible to insert a cleaned imaging plate IP into image processing section 212.

The other-end sides of unwound webs 178 are cut to the predetermined length by cutters 202, and drop into and are collected by web recovery sections 204. If a configuration were adopted in which unwound webs 178 were wound around winding cores at the downstream side of the conveyance path, the winding cores would have to be able to contact with and separate from the imaging plate conveyance path, and thus would have to be driven rollers. However, in the present embodiment, the rollers that unwind webs 178 may be drive rollers 196, so that the conveyance force of imaging plate IP can be increased.

### FOURTH EMBODIMENT

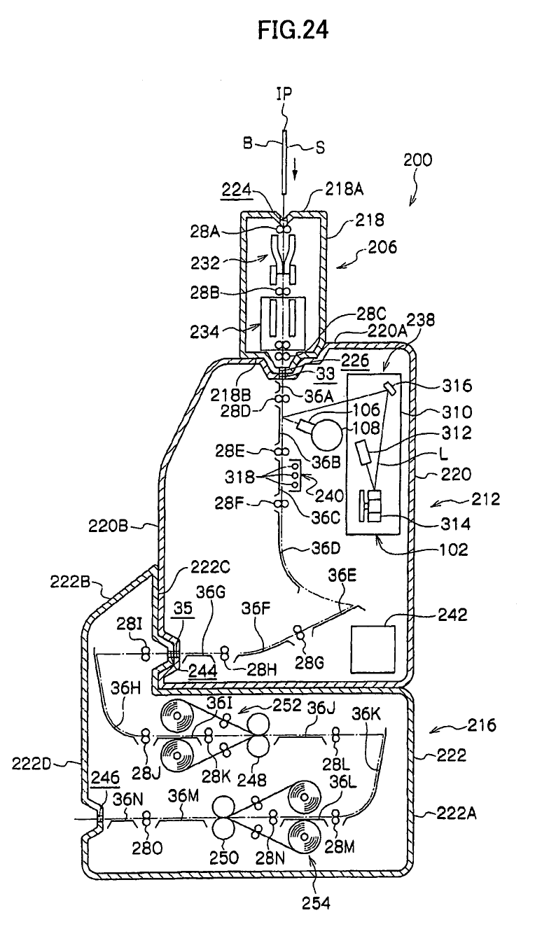

FIG. 24 is a sectional side view of a schematic structure of image reading device 200 according to the fourth embodiment. As shown in this figure, image reading device 200 has image processing section 212, image pre-processing section 206, and image post-processing section 216. Image pre-processing section 206 is contained in housing 218, and is made integral with image processing section 212 via an attachable and detachable connection between housing 218 and housing 220.

In housing 218, conveying roller pairs 28A, 28B, and 28C are disposed along the imaging plate conveyance path. Image pre-processing section 206 has protective case removal mechanism 232 provided between conveying roller pair 28A and conveying roller pair 28B, and disinfection mechanism 234 provided between conveying roller pair 28B and conveying roller pair 28C. Protective case removal mechanism 232 removes protective case 13, used for enclosing imaging plate IP, from imaging plate IP.

The operation of the present embodiment is described in the following.

When imaging plate IP enclosed within protective case 13 is inserted into housing 218 from insertion port 224, imaging plate IP is conveyed to the bottom of the device by conveying roller pair 28A, and first passes through protective case removal mechanism 232, during which protective case 13 is removed from imaging plate IP. Then, imaging plate IP without protective case 13 passes through disinfection mechanism 234, during which imaging plate IP is disinfected while stopped and held in disinfection mechanism 234 for a predetermined time. Disinfected imaging plate IP is conveyed to the bottom of the device by conveying roller pair 28C, and is discharged from housing 218 through discharge port 226 and is inserted into housing 220 through insertion port 33.

Similarly to the second and third embodiments, the X-ray image carried on imaging surface S is read by image reading mechanism 238 when imaging plate IP inserted into housing 220 passes the laser beam irradiation position in image reading mechanism 238, and the X-ray image carried on imaging surface S is erased when imaging plate IP passes the light irradiation position in residual image erasing mechanism 240. Imaging plate IP is then discharged from housing 220 and is inserted into housing 222.

Similarly to the second and third embodiments, imaging plate IP inserted into housing 222 is enclosed within protective case 13 when passing through protective case enclosure mechanism 252, and is enclosed within contamination-prevention pack 215 when passing through contamination-prevention pack enclosure mechanism 254. Imaging plate IP is then discharged from housing 222 through discharge port 246.

Here, in this embodiment, protective case 13 enclosing imaging plate IP is inserted with imaging plate IP from insertion port 224, and is removed from imaging plate IP by protective case removal mechanism 232. Therefore, efforts to remove protective case 13 from imaging plate IP are unnecessary, thereby reducing the workload of the operator. In addition, stains on imaging surface S of imaging plate IP can be further prevented since imaging plate IP can be inserted into image reading device 200 with imaging surface S protected by protective case 13.

(Protective Case Removal Mechanism 232)

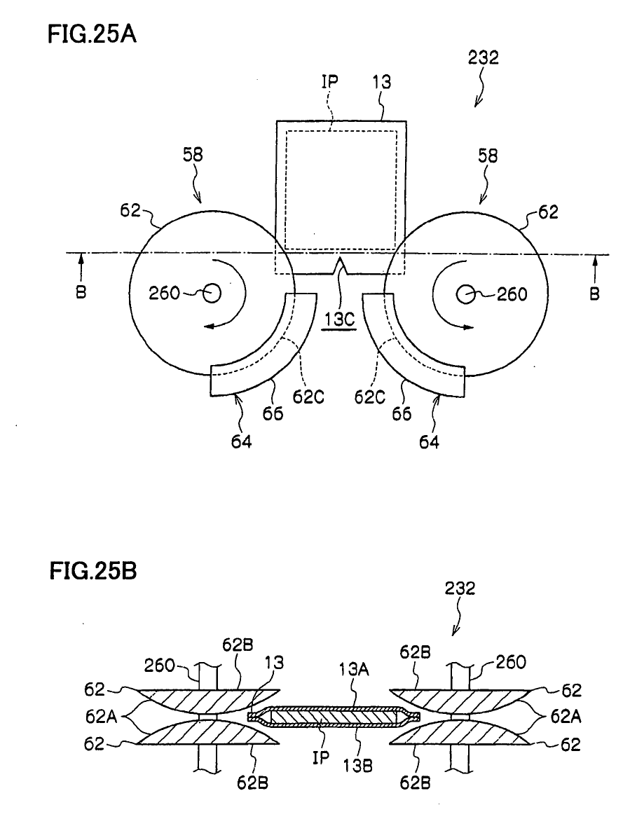

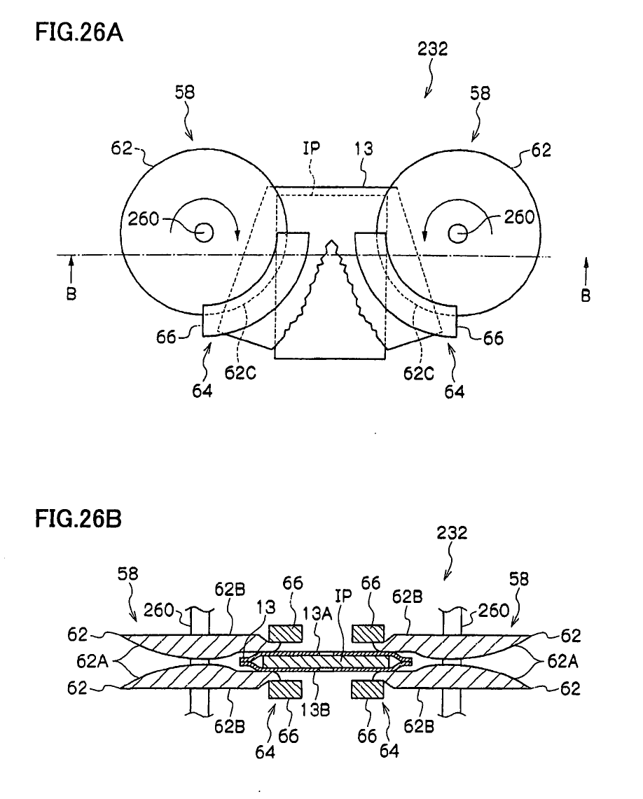

As shown in FIGS. 25A, 25B, 26A and 26B, protective case removal mechanism 232 has a pair of rotating bodies 58 disposed to face each other in the transverse direction of the imaging plate with the imaging plate conveyance path disposed therebetween. Each of rotating bodies 58 has rotating shaft 260 which is disposed at an outer side in a transverse direction of the imaging plate conveyance path and which extends along the thickness direction of the imaging plate, and a pair of bowl-shaped elastic members 62 whose axial portions are fixed to rotating shafts 260.

Each of rotating shafts 260 is rotated, by a driving unit (not shown) such as a motor, in the forward direction with respect to the conveyance direction. The pair of elastic members 62 for each rotating shaft are circular when viewed from the thickness direction of the imaging plate, and are arranged such that curved surfaces 62A face each other.

The end portion of each elastic member 62 nearer to the imaging plate conveyance path overlaps an end portion (with respect to the transverse direction of the imaging plate) of protective case 13. The end portions of a pair of elastic members 62 nearer to the imaging plate conveyance path face each other with an end portion (with respect to the transverse direction of the imaging plate) of protective case 13 disposed therebetween, wherein the pair of elastic members 62 are aligned in the thickness direction of the imaging plate. Elastic members 62 are arranged such that elastic members 62 do not contact protective case 13 when not in a state of elastic deformation.

Protective case removal mechanism 232 has a pair of pressing portions 64 disposed at both sides (in the transverse direction of the imaging plate) of the imaging plate conveyance path. Each of pressing portions 64 has a pair of pressing members 66 disposed to face each other in the direction of the thickness direction of the imaging plate with the imaging plate conveyance path disposed therebetween. The pair of pressing members 66 are circularly bent members. When viewed from the thickness direction of the imaging plate, pressing members 66 overlap peripheral portions 62C of elastic members 62, the peripheral portions 62C being nearer to the imaging plate conveyance path than the axis portions of elastic members 62 and being at the downstream side of the axis portions of elastic members 62 with respect to the conveyance direction. Pressing members 66 face each other in the thickness direction of the imaging member with peripheral portions 62C disposed therebetween.

Pressing members 66 are disposed nearer to the imaging plate conveyance path than planes 62B of elastic members 62, and elastically deform the peripheral portions 62C of elastic members 62 toward the imaging plate conveyance side. The distance between the pair of peripheral portions 62C facing each other in the direction of the thickness direction of the imaging plate is, when elastically deformed by the pair of pressing members 66, smaller than the thickness of imaging plate IP. As a result, peripheral portions 62C of the pair of elastic members 62 nip an end portion (with respect to the transverse direction of the imaging plate) of imaging plate IP and protective case 13.

Next, the operation of protective case removal mechanism 232 is described.

As shown in FIGS. 25A and 25B, when protecting case 13 enclosing imaging plate IP passes through protective case removal mechanism 232, two end portions of protective case 13 at the front side each enter the space between each pair of elastic members 62 facing each other in the thickness direction of the imaging plate.

Thereafter, as shown in FIGS. 26A and 26B, two end portions of protective case 13 at the front side are each nipped by respective pairs of elastically deformed peripheral portions 62C facing each other in the thickness direction of the imaging plate. In this state, peripheral portions 62C rotate in the forward direction with respect to the conveyance direction, and peripheral portions 62C apply a load toward an outer side in the transverse direction of the imaging plate to imaging plate IP and protective case 13.

The breakage strength of protective case 13 is set to a value such that protective case 13 is broken when an operator pulls apart both sides of notch 13C. Protective case 13 is conveyed with notch 13C at the front end. Therefore protective case 13 is broken with notch 13C serving as the cut line due to the aforementioned load from both sides of notch 13C. As a result, protective case 13 is removed from imaging plate IP.

Protective case 13, cut into two pieces, is pulled out of the imaging plate conveyance path by respective rotating bodies 58, and finally drops in and is collected by recovery sections (not shown) provided below respective rotating bodies 58.

(Modified Examples of Protective Case Removal Mechanism 232)

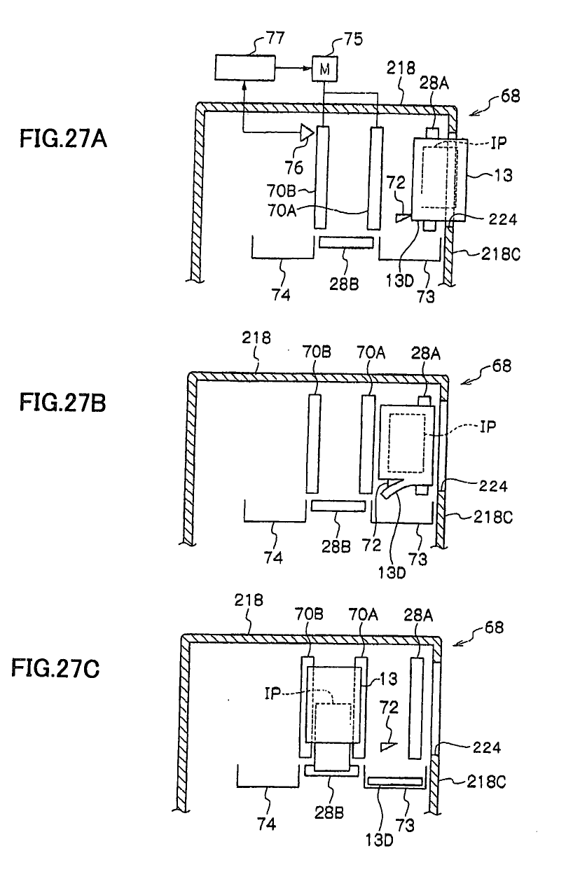

FIGS. 27A to 27C are sectional side views showing a schematic structure of protective case removal mechanism 68, which is a modified example of protective case removal mechanism 232. As shown in these figures, in image reading device 200, to which protective case removal mechanism 68 is applied, insertion port 224 is provided on side wall 218C of housing 218 along the vertical direction of the device, and upright imaging plate IP is inserted horizontally through insertion port 224. In addition, upright conveying roller pair 28A is provided in the neighborhood of insertion port 224, and conveys upright imaging plate IP horizontally.

Protective case removal mechanism 68 has conveying roller pairs 70A and 70B which are disposed substantially parallel to conveying roller pair 28A and which are disposed along the transverse direction of the imaging plate, cutter 72 which is disposed between conveying roller pair 28A and conveying roller pair 70A, cut piece recovery section 73 which is disposed below the space between conveying roller pair 28A and conveying roller pair 70A, protective case recovery section 74 which is disposed below the downstream side of conveying roller pair 70B with respect to the conveyance direction, motor (driving section) 75 which drives conveying roller pairs 70A and 70B, position detecting sensor 76 which detects the position of protective case 13 conveyed by conveying roller pairs 28A and 70A, and control section 77 which controls motor 75 based on the results of the detection by position detecting sensor 76.

Conveying roller pair 70A conveys protecting case 13 enclosing imaging plate IP and conveyed by conveying roller pair 28A to conveying roller pair 70B. The distance between the axis of conveying roller pair 70A and the axis of conveying roller pair 70B is set to a value that is approximately the same as the width (length in the transverse direction of the imaging plate) of protective case 13 conveyed by conveying roller pairs 28A and 70A, and greater than the width of imaging plate IP conveyed by the roller pairs.

Control section 77 determines the timing at which the front end portion of conveyed protective case 13 reaches the nip portion of conveying roller pair 70B based on the results of the detection by position detecting sensor 76, and stops motor 75 at that timing. When a predetermined length of time (e.g., a few seconds) has passed in this state, control section 77 drives motor 75 for a preset length of time (e.g., a few seconds).

The blade portion of cutter 72 is disposed at a position between lower joint portion 13D of protective case 13 and the lower end of imaging plate IP conveyed by conveying roller pair 28A. The axially central portion of conveying roller pair 28B is located on a straight line that runs longitudinally at the midpoint of conveying roller pair 70A and conveying roller pair 70B.

The operation of protective case removal mechanism 68 is described below.

When imaging plate IP enclosed within protective case 13 is inserted through insertion port 224 in an upright state, imaging plate IP is conveyed to conveying roller pairs 70A and 70B by conveying roller pair 28A. In this process, conveying roller pairs 70A and 70B are rotated by motor 75 driven by control section 77, thereby conveying imaging plate IP enclosed within protective case 13 into the interior of the device.

The blade portion of cutter 72 is disposed between the lower joint portion 13D of protective case 13 and the lower end of imaging plate IP conveyed by conveying roller pair 28A, and lower joint portion 13D of protective case 13 conveyed by conveying roller pair 28A is cut by cutter 72, thereby forming an opening at the lower portion of protective case 13. Joint portion 13D cut from protective case 13 by cutter 72 drops into and is collected by cut piece recovery section 73.

The position of protective case 13 conveyed by conveying roller pairs 28A and 70A is thereafter detected by position detecting sensor 76. Control section 77 determines the timing at which the front end portion of conveyed protective case 13 reaches the nip portion of conveying roller pair 70B based on the results of the detection by position detecting sensor 76, and stops motor 75 at that timing.

Since the distance between the axes of conveying roller pair 70A and conveying roller pair 70B is set to a value that is approximately the same as the width of protective case 13 conveyed by conveying roller pairs 28A and 70A, the front end portion and rear end portion of protective case 13 are respectively nipped by the nip portion of conveying roller pair 70B and the nip portion of conveying roller pair 70A. Since the distance between the axes of conveying roller pair 70A and the conveying roller pair 70B is set to a value that is greater than the width of imaging plate IP, imaging plate IP arrives at a state in which imaging plate IP is not supported by conveying roller pairs 70A and 70B.

As a result, as shown in FIG. 27C, imaging plate IP arrives at a state where imaging plate IP can fall by its own weight; therefore, imaging plate IP slips out of protective case 13 and moves toward conveying roller pair 28B, and is conveyed toward the bottom of the device by conveying roller pair 28B.

Then, control section 77 drives motor 75 so as to resume the rotation of conveying roller pairs 70A and 70B, so that protective case 13 is conveyed out of the imaging plate conveyance path. Protective case 13 subsequently drops into and is collected by protective case recovery section 74.

### FIFTH EMBODIMENT

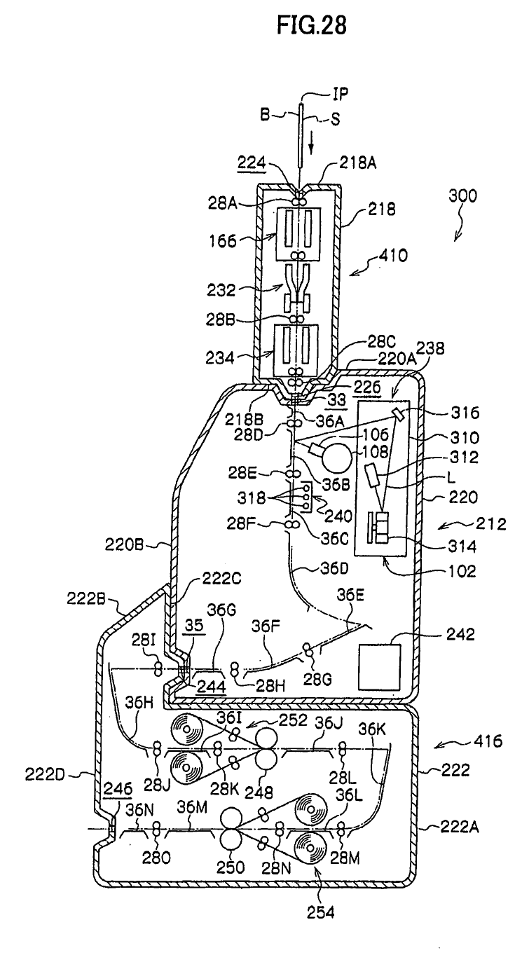

FIG. 28 is a sectional side view of a schematic structure of image reading device 300 according to the fifth embodiment. As shown in the figure, image reading device 300 has image processing section 212, image pre-processing section 410, and image post-processing section 416. Image-pre-processing section 410 is contained in housing 218, and is made integral with image processing section 212 via an attachable and detachable connection between housing 218 and housing 220.

Image pre-processing section 410 has cleaning mechanism 166 provided between conveying roller pair 28A and conveying roller pair 28B, protective case removal mechanism 232 provided between conveying roller pair 28B and conveying roller pair 28C, and disinfection mechanism 234 provided between conveying roller pair 28B and conveying roller pair 28C.

The operation of the present embodiment is described in the following.

When imaging plate IP enclosed within protective case 13 is inserted through insertion port 224 into housing 218, imaging plate IP is conveyed toward the bottom of the device by conveying roller pair 28A, and first passes through cleaning mechanism 166. At this time, protective case 13 enclosing imaging plate IP is cleaned, and contaminants, such as saliva or blood, adhered to protective case 13 are removed.

Cleaned protective case 13 and imaging plate IP enclosed within cleaned protective case 13 are conveyed toward the bottom of the device by conveying roller pair 28B, and pass through protective case removal mechanism 232, during which protective case 13 is removed from imaging plate IP.

Imaging plate IP without protective case 13 is conveyed toward the bottom of the device by conveying roller pair 28C, and is discharged from housing 218 through discharge port 226, and is inserted into housing 220 through insertion port 33.

Similarly to the second to the fourth embodiments, the X-ray image carried on imaging surface S is read by image reading mechanism 238 when imaging plate IP inserted into housing 220 passes the laser beam irradiation position in image reading mechanism 238, and the X-ray image carried on imaging surface S is erased when imaging plate IP passes the light irradiation position in residual image erasing mechanism 240. Imaging plate IP is then discharged from housing 220 and is inserted into housing 222.

Similarly to the second to the fourth embodiments, imaging plate IP inserted into housing 222 is enclosed within protective case 13 when passing through protective case enclosure mechanism 252, is enclosed within contamination-prevention pack 215 when passing through contamination-prevention pack enclosure mechanism 254, and is discharged from housing 222 through discharge port 246.

In this embodiment, since protective case 13 enclosing imaging plate IP is cleaned by cleaning mechanism 166 after being inserted into image reading device 300, adherence of contaminants, such as saliva or blood, to imaging plate IP can be prevented when protective case 13 is removed from imaging plate IP by protective case removal mechanism 232.

Therefore, image reading mechanism 238 can read an X-ray image carried on imaging plate IP that is free from adherence of saliva, blood or the like. In addition, it is not necessary for an operator to clean imaging plate IP before inserting the imaging plate into image reading device 300. Accordingly, it is possible to prevent a reduction in performance of reading X-ray images carried on imaging plates IP, and to reduce the workload of an operator.

In this embodiment, housing 218 containing cleaning mechanism 166, protective case removal mechanism 232, and disinfection mechanism 234, is attachable to and detachable from housing 220 containing image processing section 212. Therefore, even when image processing section 212 is a conventional image reading device that does not have cleaning mechanism 166, protective case removal mechanism 232, or disinfection mechanism 234, it is possible to add, as options, the cleaning mechanism, the protective case removal mechanism, and the disinfection mechanism to the conventional image reading device.

### SIXTH EMBODIMENT

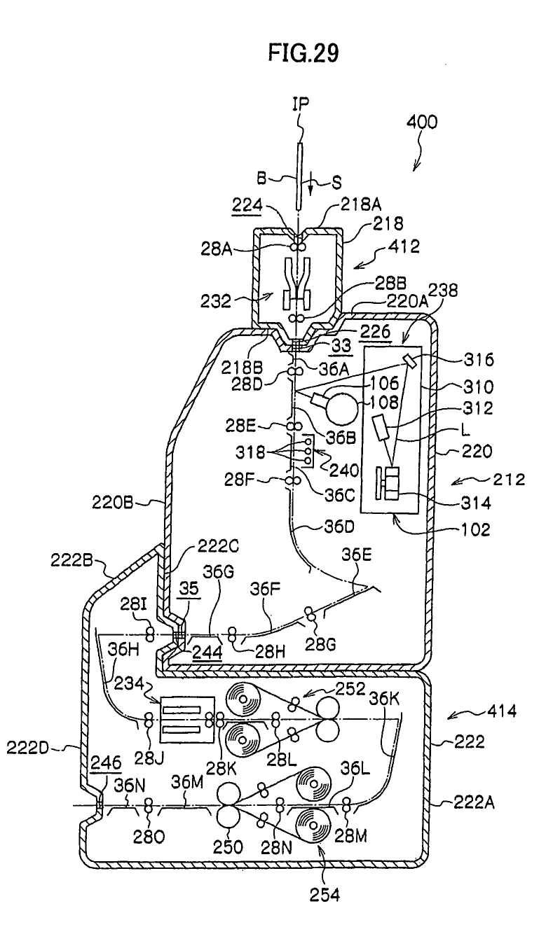

FIG. 29 is a sectional side view showing a schematic structure of image reading device 400 according to the sixth embodiment. As shown in the figure, image reading device 400 has image processing section 212, image pre-processing section 412, and image post-processing section 414. Image pre-processing section 412 is contained in housing 218, and is made integral with image processing section 212 via an attachable and detachable connection between housing 218 and housing 220. Image post-processing section 414 is contained in housing 222, and is made integral with image processing section 212 via an attachable and detachable connection between housing 222 and housing 220.

Image pre-processing section 412 has protective case removal mechanism 232 provided between conveying roller pair 28A and conveying roller pair 28B. Image post-processing section 414 has disinfection mechanism 234 provided between conveying roller pair 28J and conveying roller pair 28K, protective case enclosure mechanism 252 provided between conveying roller pair 28K and conveying guide 36K, and contamination-prevention pack enclosure mechanism 254 provided between conveying roller pair 28M and conveying guide 36M.

The operation of the present embodiment is explained in the following.

When imaging plate IP enclosed within protective case 13 is inserted into housing 218 through insertion port 224, imaging plate IP is conveyed toward the bottom of the device by conveying roller pair 28A and first passes through protective case removal mechanism 232, at which time protective case 13 is removed from imaging plate IP.

Imaging plate IP, having had protective case 13 removed therefrom, is conveyed toward the bottom of the device by conveying roller pair 28B, passes through discharge port 226 and is discharged from housing 218 and, at the same time, passes through insertion port 33 and is inserted into housing 220.

Similarly to the second to fifth embodiments, the X-ray image carried on imaging surface S is read by image reading mechanism 238 when imaging plate IP, having been inserted into housing 220, passes the laser beam irradiation position in image reading mechanism 238, and the X-ray image carried on imaging surface S is erased when imaging plate IP passes the light irradiation position in residual image erasing mechanism 240. Imaging plate IP is then discharged from housing 220 and is inserted into housing 222.

Imaging plate IP, having been inserted into housing 222, first passes through disinfection mechanism 234. Here, imaging plate IP stops inside disinfection mechanism 234 for a predetermined time and is sterilized and disinfected. Then, sterilized and disinfected imaging plate IP is conveyed by conveying roller pair 28K toward the rear of the device. After this, imaging plate IP passes through protective case enclosure mechanism 252 and is enclosed in protective case 13, then passes through contamination-prevention pack enclosure mechanism 254 and is enclosed in contamination-prevention pack 215 as well as protective case 13, and is discharged from housing 222.

In the present embodiment, imaging plate IP carrying an X-ray image is sterilized and disinfected by disinfection mechanism 234 after the X-ray image is read by image reading mechanism 238 and after the X-ray image is erased by residual image erasing mechanism 240.

Namely, since reading of the X-ray image is performed by image reading mechanism 238 before sterilization and disinfection of imaging plate IP is performed by disinfection mechanism 234, it is possible to suppress lengthening of the time required between imaging plate IP being inserted inside image reading device 400 and the X-ray image being displayed on a monitor. Further, the workload of an operator is decreased because it is unnecessary for the operator to disinfect imaging plate IP.

Further, in the present embodiment, housing 222, which accommodates disinfection mechanism 234, is attachably and detachably connected to housing 220, which accommodates image reading mechanism 238 and residual image erasing mechanism 240. As a result, even when image processing section 212 is a conventional image reading device that is not equipped with disinfection mechanism 234, it is possible to optionally add a disinfection function to the conventional image reading device.

### SEVENTH EMBODIMENT

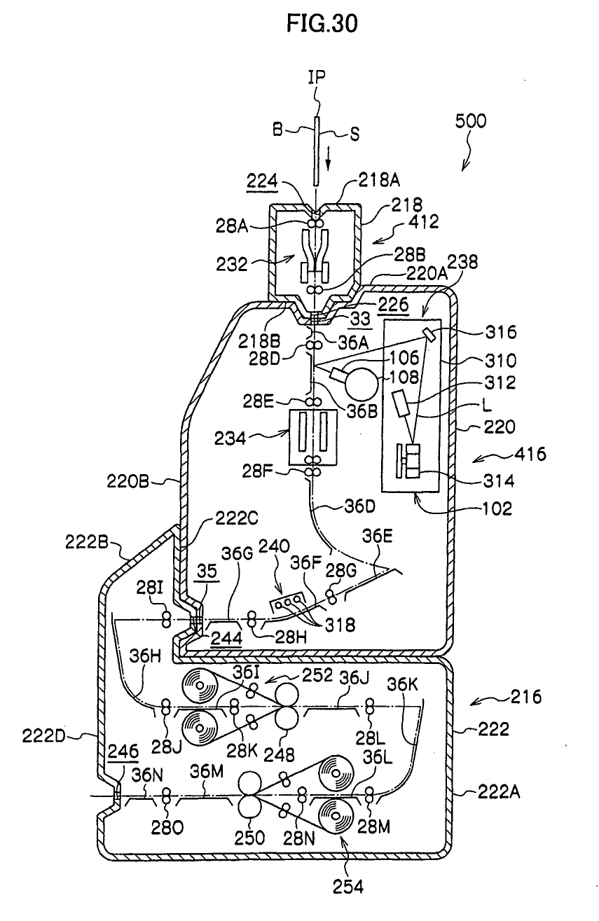

FIG. 30 shows a sectional side view of a schematic structure of image reading device 500 according to a seventh embodiment. As shown in the drawing, image reading device 500 is provided with image processing section 416, image pre-processing section 412 and image post-processing section 216.

Image processing section 416 is provided with disinfection mechanism 234 disposed between conveying roller pair 28E and conveying roller pair 28F, and with residual image erasing mechanism 240 disposed between conveying roller pair 28G and conveying roller pair 28H.

The operation of the present embodiment is explained in the following.

When imaging plate IP enclosed within protective case 13 is inserted into housing 218 through insertion port 224, imaging plate IP is conveyed toward the bottom of the device by conveying roller pair 28A and first passes through protective case removal mechanism 232, at which time protective case 13 is removed from imaging plate IP.

Imaging plate IP, having had protective case 13 removed therefrom, is conveyed toward the bottom of the device by conveying roller pair 28B, passes through discharge port 226 and is discharged from housing 218 and, at the same time, passes through insertion port 33 and is inserted into housing 220.

Imaging plate IP, having been inserted into housing 220, is conveyed by conveying roller pair 28D, passes the laser beam irradiation position in image reading mechanism 238 and the X-ray image carried on imaging surface S is read by image reading mechanism 238. The X-ray image read by image reading mechanism 238 is displayed at a monitor.

Imaging plate IP, having passed the laser beam irradiation position in image reading mechanism 238, is conveyed by conveying roller pair 28E toward the bottom of the device and passes disinfection mechanism 234. Here, imaging plate IP stops inside disinfection mechanism 234 for a predetermined time and is sterilized and disinfected. Then, sterilized and disinfected imaging plate IP is conveyed toward the bottom of the device by conveying roller pair 28F and is then guided toward conveying roller pair 28G by conveying guides 36D, 36E. Here, the forward end and the rear end of imaging plate IP in the direction of conveyance are reversed by conveying guides 36D, 36E and imaging surface S is faced upward.

After this, imaging plate IP is conveyed by conveying roller pair 28G in a state in which imaging surface S faces upward, passes the light irradiation position in residual image erasing mechanism 240, and the X-ray image carried on imaging surface S is erased.

Then, imaging plate IP, having had the X-ray image erased therefrom, is conveyed toward the front of the device by conveying roller pair 28H and is discharged from housing 220 through discharge port 35 and inserted into housing 222 through insertion port 244.

Similarly to the second embodiment, imaging plate IP, having been inserted into housing 222, is enclosed in protective case 13 when passing through protective case enclosure mechanism 252 and is enclosed in contamination-prevention pack 215 together with protective case 13 when passing through contamination-prevention pack enclosure mechanism 254, and is then discharged from housing 222.

In the present embodiment, similarly to the sixth embodiment, imaging plate IP carrying an X-ray image is sterilized and disinfected by disinfection mechanism 234 after the X-ray image is read by image reading mechanism 238.

Namely, since reading of the X-ray image is performed by image reading mechanism 238 before sterilization and disinfection of imaging plate IP is performed by disinfection mechanism 234, it is possible to suppress lengthening of the time required between imaging plate IP being inserted inside image reading device 500 and the X-ray image being displayed on a monitor. Further, the workload of an operator is decreased because it is unnecessary for the operator to disinfect imaging plate IP.

### Eighth Embodiment

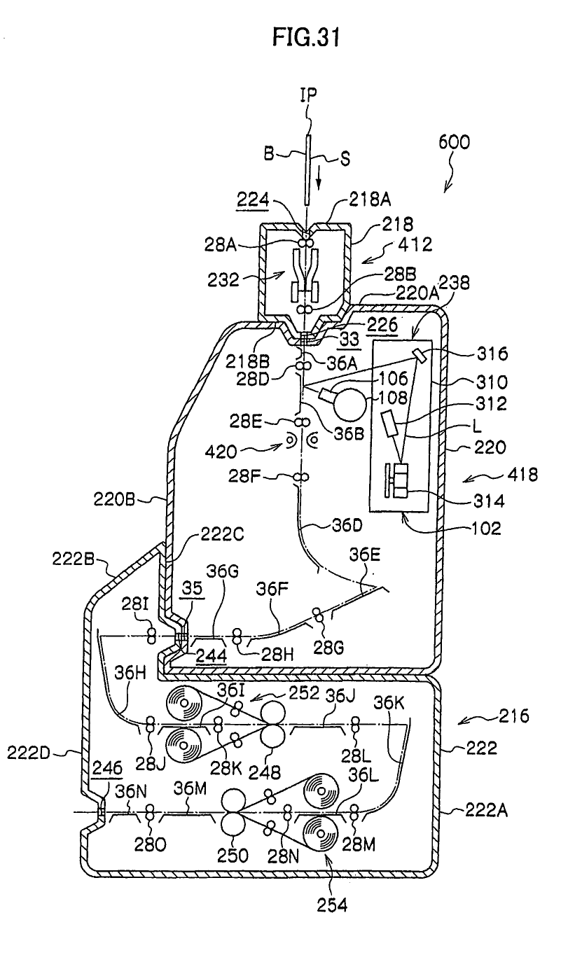

FIG. 31 shows a sectional side view of a schematic structure of image reading device 600 according to an eighth embodiment. As shown in the drawing, image reading device 600 is provided with image processing section 418, image pre-processing section 412 and image post-processing section 216.

Image processing section 418 is provided with erasing and disinfection mechanism 420, as an erasing and disinfection unit, between conveying roller pair 28E and conveying roller pair 28F. Erasing and disinfection mechanism 420 irradiates UV light (ultraviolet rays) onto imaging surface S and rear surface B of imaging plate IP and erases the X-ray image carried by imaging plate IP at the same time as sterilizing and disinfecting imaging plate IP.

The operation of the present embodiment is explained in the following.

When imaging plate IP enclosed within protective case 13 is inserted into housing 218 through insertion port 224, imaging plate IP is conveyed toward the bottom of the device by conveying roller pair 28A and first passes through protective case removal mechanism 232, at which time protective case 13 is removed from imaging plate IP.

Imaging plate IP, having had protective case 13 removed therefrom, is conveyed toward the bottom of the device by conveying roller pair 28B, passes through discharge port 226 and is discharged from housing 218 and, at the same time, passes through insertion port 33 and is inserted into housing 220.

Imaging plate IP, having been inserted into housing 220, is conveyed by conveying roller pair 28D, passes the laser beam irradiation position in image reading mechanism 238 and the X-ray image carried on imaging surface S is read by image reading mechanism 238. The X-ray image read by image reading mechanism 238 is displayed at a monitor.

Imaging plate IP, having passed the laser beam irradiation position in image reading mechanism 238, is conveyed by conveying roller pair 28E toward the bottom of the device and passes the UV light irradiation position of erasing and disinfection mechanism 420. Here, imaging plate IP stops inside erasing and disinfection mechanism 420 for a predetermined time, the X-ray image is erased, and imaging plate IP is sterilized and disinfected. Then, sterilized and disinfected imaging plate IP having had the X-ray image erased therefrom is conveyed toward the bottom of the device by conveying roller pair 28F and is then guided toward conveying roller pair 28G by conveying guides 36D, 36E. Here, the forward end and the rear end of imaging plate IP in the direction of conveyance are reversed by conveying guides 36D, 36E and imaging surface S is faced upward.

After this, imaging plate IP is conveyed by conveying roller pairs 28G, 28H in a state in which imaging surface S faces upward, and is discharged from housing 220 through discharge port 35 and inserted into housing 222 through insertion port 244.

Similarly to the second embodiment, imaging plate IP, having been inserted into housing 222, is enclosed in protective case 13 when passing through protective case enclosure mechanism 252 and is enclosed in contamination-prevention pack 215 together with protective case 13 when passing through contamination-prevention pack enclosure mechanism 254, and is then discharged from housing 222.

In the present embodiment, similarly to the sixth and seventh embodiments, sterilization and disinfection of imaging plate IP carrying an X-ray image is performed after the X-ray image is read by image reading mechanism 238.

Namely, since reading of the X-ray image is performed by image reading mechanism 238 before sterilization and disinfection of imaging plate IP is performed by erasing and disinfection mechanism 420, it is possible to suppress lengthening of the time required between imaging plate IP being inserted inside image reading device 600 and the X-ray image being displayed on a monitor. Further, the workload of an operator is decreased because it is unnecessary for the operator to disinfect imaging plate IP.

Further, in the present embodiment, since disinfectant treatment is carried out by erasing and disinfection mechanism 420 during erasing of the image by erasing and disinfection mechanism 420, the time required until imaging plate IP is discharged can be shortened.

In addition, in the present embodiment, it is possible to reduce the space occupied by the erasing unit and the disinfection unit by installing an integrated erasing and disinfection unit in the form of erasing and disinfection mechanism 420, and thus to reduce the size of image reading device 600.

(Erasing and Disinfection Mechanism 420)

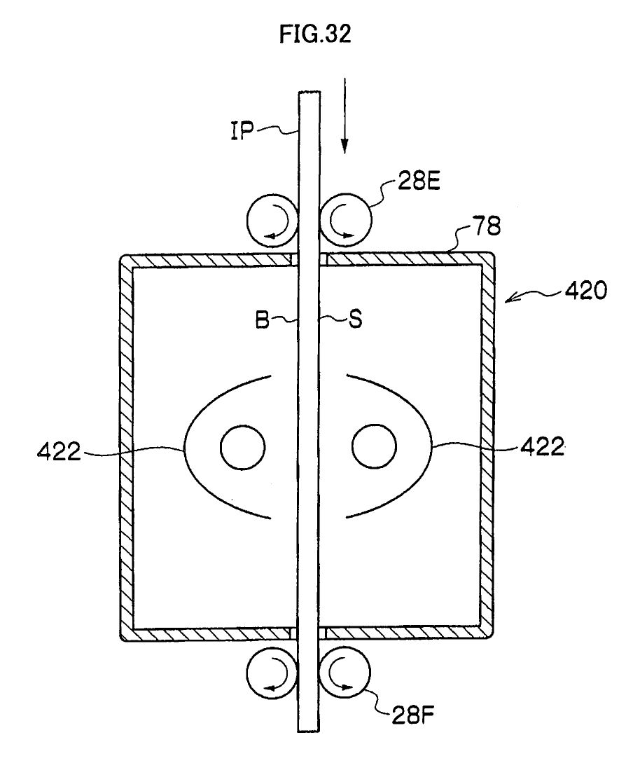

FIG. 32 shows a side sectional view of the schematic configuration of erasing and disinfection mechanism 420. As shown in the drawing, erasing and disinfection mechanism 420 is provided with housing 78 and a pair of UV light sources 422. The pair of UV light sources 422 face each other in an imaging plate thickness direction with the imaging plate conveyance path interposed therebetween, and irradiate UV light toward the imaging plate conveyance path.

The operation of erasing and disinfection mechanism 420 is explained in the following.

When imaging plate IP, having had the X-ray image read by image reading mechanism 238, is conveyed toward the bottom of the device by conveying roller pair 28E, UV light is irradiated from the pair of UV light sources 422 toward imaging surface S and rear surface B of imaging plate IP. As a result, the X-ray image carried on from imaging surface S of imaging plate IP is erased and imaging surface S and rear surface B of imaging plate IP are sterilized and disinfected.

In the foregoing, specific embodiments of the present invention have been explained in detail; however, the present invention is not limited to these embodiments and it will be evident to one skilled in the art that a variety of different embodiments are possible within the scope of the present invention.

The present invention aims to solve the conventional problems. That is, the present invention aims to provide an image reading device having a disinfectant unit that can uniformly and effectively disinfect an imaging medium and a protective member that covers at least the imaging surface of the imaging medium.

The present invention provides an image reading device provided with a disinfection unit that can uniformly and effectively disinfect an imaging medium and a protective member that covers at least the imaging surface of the imaging medium.

All publications, patent applications, and technical standards mentioned in this specification are herein incorporated by reference to the same extent as if each individual publication, patent application, or technical standard was specifically and individually indicated to be incorporated by reference.

What is claimed is:

1. An image reading device, comprising:
a disinfection unit that administers a disinfection treatment to an imaging medium carrying a radiation image or to a protective member covering at least an imaging surface of the imaging medium; and
an image reading unit that reads the radiation image carried by the imaging medium either after or before the disinfection treatment by the disinfection unit.

2. The image reading device of claim 1, wherein the disinfection treatment is at least one treatment selected from the group consisting of heat treatment, ultraviolet ray irradiation treatment, chemical coating treatment and gas treatment.

3. The image reading device of claim 2, wherein:
the imaging medium is a radiation image conversion panel; and
the disinfection treatment by the disinfection unit is heat treatment, and the heat treatment comprises heating the radiation image conversion panel at 60° C. to 200° C. for 1 second to 10 minutes.

4. The image reading device of claim 1, wherein the imaging medium is a radiation image conversion panel having a protective layer with a thermal shrinkage rate of 1% or less at 150° C. for 30 minutes.

5. The image reading device of claim 4, wherein the protective layer of the radiation image conversion panel is subjected to heat treatment at 60° C. or above at either or both of before and during formation thereof.

6. The image reading device of claim 2, wherein the disinfection treatment by the disinfection unit is heat treatment and the disinfection unit is equipped with a temperature control unit.

7. The image reading device of claim 2, wherein the disinfection treatment by the disinfection unit is heat treatment and the heat treatment comprises heating with either or both of an infrared heater and a far-infrared heater.

8. The image reading device of claim 2, wherein the disinfection treatment by the disinfection unit is ultraviolet ray irradiation treatment, and irradiation energy of ultraviolet rays in the ultraviolet ray irradiation treatment is 0.04 J/cm² or above.

9. The image reading device of claim 1, further comprising:
an insertion port through which the imaging medium is inserted;
a conveying unit that conveys the imaging medium that has been inserted through the insertion port;
a residual image erasing unit that erases from the imaging medium a residual image of the radiation image carried by the imaging medium after the radiation image has been read by the image reading unit; and
a discharge port through which the imaging medium is discharged after the residual image is erased by the residual image erasing unit, wherein:
the disinfection unit disinfects the imaging medium that has been inserted through the insertion port; and
the image reading unit reads the radiation image carried by the imaging medium from the imaging medium that has been disinfected by the disinfection unit.

10. The image reading device of claim 9, wherein the discharge port is separated from the insertion port.

11. The image reading device of claim 9, further comprising a device housing that accommodates at least the image reading unit and the residual image erasing unit and that the disinfection unit is freely attachable to and detachable from.

12. The image reading device of claim 9, further comprising a protective member removal unit that is disposed at a downstream side of the insertion port in a direction of conveyance and at an upstream side of the disinfection unit in the direction of conveyance, and that removes the protective member from the imaging medium, wherein the insertion port is configured such that the protective member can be inserted together with the imaging medium.

13. The image reading device of claim 9, further comprising a protective member attachment unit that is disposed at a downstream side of the residual image erasing unit in a direction of conveyance, and that attaches the protective member to the imaging medium.

14. The image reading device of claim 13, further comprising a pack enclosure unit that is disposed at a downstream side of the protective member attachment unit in the direction of conveyance, and that encloses the imaging medium within a contamination-prevention pack that prevents adhesion of contaminants to the imaging medium.

15. The image reading device of claim 9, further comprising:
a partition member that partitions the inside of the device into a disinfection chamber accommodating the disinfection unit and an image processing chamber accommodating the image reading unit; and
a chamber pressure maintenance unit that maintains the chamber pressure of the image processing chamber at a higher pressure than the chamber pressure of the disinfection chamber.

16. The image reading device of claim 1, further comprising:
an insertion port through which the imaging medium is inserted;
a conveying unit that conveys the imaging medium that has been inserted through the insertion port;
a residual image erasing unit that is disposed at a downstream side of the image reading unit in a direction of conveyance and that erases a residual image of the radiation image carried by the imaging medium; and
a discharge port through which the imaging medium is discharged, that is disposed at a downstream side of the residual image erasing unit and the disinfection unit in the direction of conveyance, and that is different from the insertion port, wherein:
the image reading unit is disposed at a downstream side of the insertion port in the direction of conveyance; and
the disinfection unit is disposed at a downstream side of the image reading unit in the direction of conveyance.

17. The image reading device of claim 16, wherein the disinfection treatment by the disinfection unit is performed during residual image erasing processing by the residual image erasing unit.

18. The image reading device of claim 16, wherein the residual image erasing unit is integrated with the disinfection unit.

19. The image reading device of claim 16, further comprising a device housing that accommodates at least the image reading unit and that the disinfection unit is freely attachable to and detachable from.

20. The image reading device of claim 16, further comprising a protective member removal unit that is disposed at a downstream side of the insertion port in the direction of conveyance and at an upstream side of the image reading unit in the direction of conveyance, and that removes the protective member from the imaging medium, wherein the insertion port is configured such that the protective member can be inserted together with the imaging medium.

21. The image reading device of claim 16, further comprising a protective member attachment unit that is disposed at a downstream side of the residual image erasing unit and the disinfection unit in the direction of conveyance, and that attaches the protective member to the imaging medium.

22. The image reading device of claim 21, further comprising a pack enclosure unit that is disposed at a downstream side of the protective member attachment unit in the direction of conveyance, and that encloses the imaging medium within a contamination-prevention pack that prevents adhesion of contaminants to the imaging medium.

23. The image reading device of claim 1, further comprising:
an insertion port through which the imaging medium is inserted;
a conveying unit that conveys the imaging medium that has been inserted through the insertion port;
a cleaning unit that cleans the imaging medium that has been inserted through the insertion port;
a residual image erasing unit that erases from the imaging medium a residual image of the radiation image carried by the imaging medium after the radiation image has been read by the image reading unit; and
a discharge port through which the imaging medium is discharged after the residual image is erased by the residual image erasing unit, wherein:
the image reading unit reads the radiation image carried by the imaging medium from the imaging medium that has been disinfected by the cleaning unit.

24. The image reading device of claim 23, further comprising a protective member removal unit that removes the protective member from the imaging unit after the imaging medium has been inserted through the insertion port and before the imaging medium has been cleaned by the cleaning unit, wherein the insertion port is configured such that the protective member can be inserted together with the imaging medium.

25. The image reading device of claim 1, further comprising:
an insertion port through which the imaging medium is inserted in a state in which at least the imaging surface is protected by the protective member;
a conveying unit that conveys the imaging medium that has been inserted through the insertion port;
a cleaning unit that cleans the protective member that has been inserted through the insertion port;
a protective member removal unit that removes from the imaging medium the protective member that has been cleaned by the cleaning unit; and
a residual image erasing unit that erases from the imaging medium a residual image of the radiation image carried by the imaging medium after the radiation image has been read by the image reading unit, wherein
the image reading unit reads from the imaging medium the radiation image carried by the imaging medium after the protective member has been removed by the protective member removal unit.

26. The image reading device of claim 23, wherein the discharge port is separated from the insertion port.

27. The image reading device of claim 23, further comprising a device housing that accommodates at least the image reading unit and an image removal unit, and that the cleaning unit is freely attachable to and detachable from.

28. The image reading device of claim 23, wherein the disinfection unit is disposed at a downstream side of the cleaning unit in a direction of conveyance.

29. The image reading device of claim 28, further comprising a protective member attachment unit that is disposed at a downstream side of the disinfection unit in a direction of conveyance, and that attaches the protective member to the imaging medium.

30. The image reading device of claim 29, further comprising a pack enclosure unit that is disposed at a downstream side of the protective member attachment unit in the direction of conveyance, and that encloses the imaging medium within a contamination-prevention pack that prevents adhesion of contaminants to the imaging medium.
